(19) United States
(12) Patent Application Publication
(10) Pub. No.: US 2026/0174513 A1
(43) Pub. Date: Jun. 25, 2026

(54) INSTRUMENT STERILE ADAPTER DEVICE FOR SURGICAL ROBOT SYSTEM

(71) Applicant: LivsMed Inc., Seongnam-si (KR)

(72) Inventors: Jae Hoon SHIN, Gwangju-si (KR); Gyu Dong JEON, Seoul (KR); Young Jae SONG, Seongnam-si (KR); Sera KIM, Suwon-si (KR); Young Wan KIM, Seongnam-si (KR)

(73) Assignee: LivsMed Inc., Seongnam-si (KR)

(21) Appl. No.: 19/394,005

(22) Filed: Nov. 19, 2025

(30) Foreign Application Priority Data
Dec. 23, 2024 (KR) 10-2024-0194071

(51) Int. Cl. A61B 34/30; A61B 34/00

(52) U.S. Cl. CPC A61B 34/30; A61B 34/71

(57) ABSTRACT

Provided is an instrument sterile adapter device for a surgical robot system. The instrument sterile adapter device includes: a sterile adapter body having a carriage coupling surface and an instrument coupling surface, wherein the carriage coupling surface is configured to be coupled to an instrument carriage, and the instrument coupling surface is disposed opposite to the carriage coupling surface and configured to be coupled to a surgical instrument; at least one coupler mounted on the sterile adapter body, and configured to transfer rotational power from the instrument carriage to the surgical instrument; and a fixed cover detachably coupled to the sterile adapter body and configured to restrain rotation of the at least one coupler while the fixed cover is mounted on the sterile adapter body.

### CROSS-REFERENCE TO RELATED APPLICATION

This application claims the benefit of and priority to Korean Patent Application No. 10-2024-0194071, filed on Dec. 23, 2024, the entire disclosure(s) of which is hereby incorporated herein by reference in its entirety.

### BACKGROUND

### 1. Field

The present disclosure relates to a surgical robot system, and more specifically, but not limitedly, to an instrument sterile adapter device mounted on a surgical instrument carriage of the surgical robot system.

### 2. Description of the Related Art

In medical terms, surgery refers to the treatment of a disease by using medical devices to cut, slit, or manipulate skin, a mucous membrane, or other tissue. In particular, open surgery of cutting and opening the skin of a surgical site to treat, reshape, or remove organs therein causes bleeding, side effects, pain to a patient, and scars. Accordingly, recently, surgery using a robot or surgery performed by inserting only a medical device, for example, a laparoscope, a surgical instrument, a microsurgical microscope, or the like, in the body by forming a predetermined hole in the skin, has been spotlighted as an alternative.

Herein, a surgical robot refers to a robot that has a function of replacing a surgical action performed by a surgeon. The surgical robot may operate more accurately and precisely as compared with a human and enable remote surgery.

A surgical robot system is generally composed of a master robot and a slave robot. When a surgical operator manipulates a control lever (for example, a handle) provided on the master robot, a surgical instrument coupled to or held by a robot arm on the slave robot is manipulated to perform surgery.

In this regard, a surgical instrument carriage is provided on a robot arm of the slave robot to mount a surgical instrument. The surgical instrument carriage may include a motor for operating a surgical instrument, more specifically an end tool, and may be referred to as, but not limited to, a ‘motor pack’, for example. For a surgical procedure, it is required to ensure that not only the surgical instrument used in the surgery, but also the slave robot including the motor pack and the robot arm located at a rear side of the motor pack are sterilized. However, such a surgical instrument carriage includes electronic equipment such as, for example, a motor, and thus, there is a high risk of damage or breakage during the sterilization process when sterilization is performed using steam, heat, or pressure.

To address these issues, a method of maintaining a sterile state by covering the slave robot at a rear side of the surgical instrument carriage using a drape has been proposed. In particular, since the surgical instrument carriage is required to maintain a power-transmitting connection relationship with the surgical instrument, a structure in which an instrument sterile adapter device with the drape fastened thereto is first coupled to the surgical instrument carriage, and then the driving part of the surgical instrument is fastened to the instrument sterile adapter device, so that the rotational power is transmitted in the order of the surgical instrument carriage, the instrument sterile adapter device, and the surgical instrument is widely utilized.

The aforementioned background technology corresponds to technical information that has been possessed by the present inventor(s) in order to derive the present disclosure or which has been acquired in the process of deriving the present disclosure, and may not necessarily be regarded as well-known technology which had been known to the public prior to the filing of the present disclosure.

### SUMMARY

An exemplary aspect of the present disclosure is directed to providing an instrument sterile adapter device capable of being mounted on a surgical instrument carriage provided in a surgical robot system. In addition, an aspect of the present disclosure is directed to providing a computer-readable recording medium recording a program for executing the method on a computer.

The aspects of the present disclosure are not limited to those mentioned above, and other aspects and benefits not mentioned may be understood from the following description and may be more clearly understood by the embodiments of the present disclosure. In addition, the aspects and benefits to be solved by the present disclosure may be realized by the means indicated in the scope of claims and combinations thereof.

An instrument sterile adapter device for a surgical robot system according to an embodiment of the present disclosure may include: a sterile adapter body having a carriage coupling surface and an instrument coupling surface, wherein the carriage coupling surface is configured to be coupled to an instrument carriage, and the instrument coupling surface is disposed opposite to the carriage coupling surface and configured to be coupled to a surgical instrument; at least one coupler mounted on the sterile adapter body, and configured to transfer rotational power from the instrument carriage to the surgical instrument; and a fixed cover detachably coupled to the sterile adapter body and configured to restrain rotation of the at least one coupler while the fixed cover is mounted on the sterile adapter body.

According to an aspect, the instrument sterile adapter device may be configured to be coupled to the instrument carriage in a state where the fixed cover is mounted on the instrument sterile adapter device, and the fixed cover would be configured to be removed after at least one driver of the instrument carriage is coupled to the at least one coupler.

According to an aspect, the at least one coupler may include at least one driver coupling unit protruded from the carriage coupling surface, the at least one driver of the instrument carriage may include at least one coupler coupling unit disposed correspondingly to the at least one driver coupling unit, and the at least one driver coupling unit and the at least one coupler coupling unit may be configured to be mutually engaged by rotation of the at least one driver so as to correspond to each other.

According to an aspect, the at least one driver of the instrument carriage may be configured to be rotated by at least one motor provided in the instrument carriage.

According to an aspect, the at least one coupler may include at least one stopper coupling unit protruded from the instrument coupling surface, and the fixed cover may include at least one stopper disposed correspondingly to the at least one stopper coupling unit configured to restrain rotation of the coupler.

According to an aspect, the at least one stopper coupling unit may include at least one protruding stopper coupling unit protruding from the instrument coupling surface.

According to an aspect, the at least one stopper may include at least one groove-shaped stopper configured to accommodate the at least one protruding stopper coupling unit.

According to an aspect, the at least one stopper may include: a step portion formed at a first position in a rotational direction of the coupler; and a tapered portion having a height gradually decreasing from the step portion along the rotational direction of the coupler, wherein the at least one protruding stopper coupling unit may be configured to allow a first direction rotation of the coupler by moving along the tapered portion, and to restrain a second direction rotation of the coupler opposite to the first direction by causing the at least one protruding stopper coupling unit to be caught by the step portion.

According to an aspect, the at least one stopper coupling unit may include at least one groove-shaped stopper coupling unit, and the at least one stopper may include at least one protruding stopper configured to be inserted into the at least one groove-shaped stopper coupling unit.

According to an aspect, the at least one stopper coupling unit may be at least one driving pulley coupling unit configured to be coupled to at least one driving pulley of the surgical instrument in a state where the surgical instrument is coupled to the instrument sterile adapter device.

According to an aspect, the at least one driving pulley configured to control an operation of the surgical instrument based on a rotational power of at least one driver transmitted through the coupler.

According to an aspect, the sterile adapter body may include at least one instrument coupling latch protruded from the instrument coupling surface, and the at least one instrument coupling latch may have at least one instrument latch receiving groove formed such that at least one instrument latch provided on the surgical instrument is seated in a state where the surgical instrument is coupled to the instrument sterile adapter device.

According to an aspect, the fixed cover may include: at least one instrument coupling latch hole configured to allow the at least one instrument coupling latch to pass through; and at least one fixed cover latch seated in the instrument latch receiving groove in a state where the fixed cover is coupled to the sterile adapter body.

According to an aspect, the fixed cover further includes at least one fixed cover latch support extending in a direction opposite to the sterile adapter body, and the at least one fixed cover latch may be formed on the at least one fixed cover latch support.

According to an aspect, the at least one instrument coupling latch may have a length such that the at least one instrument coupling latch protrudes further than the at least one fixed cover latch support in a state where the fixed cover is coupled to the sterile adapter body.

According to an aspect, the sterile adapter body may include: a passage through which at least one sterile adapter latch support extending from the instrument carriage passes through the carriage coupling surface; and a sterile adapter latch receiving groove in which at least one sterile adapter latch coupled to the at least one sterile adapter latch support is seated in a state where the sterile adapter body is coupled to the instrument carriage.

According to an aspect, the sterile adapter latch receiving groove may be formed on a side surface of the sterile adapter body.

According to an aspect, the sterile adapter latch support may be connected to a sterile adapter release button provided on the instrument carriage, and is configured such that the sterile adapter latch disengages from the sterile adapter latch receiving groove in response to a pressing of the sterile adapter release button.

In addition, another method for implementing the present disclosure, another system, and a computer-readable recording medium storing a computer program for executing the method may be further provided.

Other aspects, features, and advantages in addition to those described above will become apparent from the following drawings, claims, and detailed description of the present disclosure.

In an embodiment of the present disclosure, the sterile adapter body has a carriage coupling surface for coupling to an instrument carriage and an instrument coupling surface disposed on an opposite side of the carriage coupling surface for coupling to a surgical instrument, and at least one coupler is mounted on the sterile adapter body to transmit rotational power from the instrument carriage to the surgical instrument. Herein, a fixed cover may be provided, the fixed cover being configured to be detachably coupled to the sterile adapter body and, when mounted on the sterile adapter body, to restrain rotation of the at least one coupler. Accordingly, since a coupler rotation stopper structure for coupling between a coupler of the instrument sterile adapter device and a driver of the surgical instrument carriage need not be provided on the instrument sterile adapter device, the thickness of the instrument sterile adapter device can be reduced, and thus the length of the surgical instrument and the surgical robot can also be reduced. Accordingly, it provides a more advantageous effect in increasing the rigidity of the surgical instrument and the surgical robot. In addition, compared to when the coupler rotation stopper structure is provided on the instrument sterile adapter device, the axial dimensional deviation of the coupler can be more fully managed.

In addition, according to an aspect of the present disclosure, a fastening structure for coupling between the surgical instrument carriage and the instrument sterile adapter device and a fastening structure for coupling between the instrument sterile adapter device and the surgical instrument can be separately provided. Accordingly, not only can the detachment of the instrument sterile adapter from the surgical instrument carriage be performed quickly and easily, but also the instrument sterile adapter device can be advantageously removed from the surgical instrument carriage even when the fastening structure for coupling with the surgical instrument provided on the instrument sterile adapter device is damaged.

The benefits of the present disclosure are not limited to those mentioned above, and other benefits not mentioned may be clearly understood by those skilled in the art from the following description.

### BRIEF DESCRIPTION OF THE DRAWINGS

FIG. 1 is a diagram for explaining an example of a system for driving a surgical instrument according to an embodiment.

FIG. 2A is a configuration diagram illustrating an example of a user terminal according to an embodiment.

FIG. 2B is a configuration diagram illustrating an example of a server according to an embodiment.

FIG. 3 is a conceptual diagram illustrating a surgical robot system according to an embodiment.

FIG. 4 is a block diagram illustrating the internal configuration of the surgical robot system of FIG. 3.

FIG. 5 is a perspective view of a slave robot of the surgical robot system of FIG. 3 and a surgical instrument mounted thereon.

FIG. 6 is a perspective view of a modular slave robot and a surgical instrument mounted thereon according to an aspect of the surgical robot system of FIG. 3.

FIG. 7 is a diagram illustrating a state in which the instrument case is removed from FIG. 6.

FIG. 8 is a perspective view of a modular slave robot and a laparoscopic surgical camera mounted thereon according to another aspect of the surgical robot system of FIG. 3.

FIG. 9 is a diagram illustrating a state in which the surgical instrument is removed from the slave robot of FIG. 6.

FIG. 10 is a perspective view of another example of a modular slave robot and a surgical instrument mounted thereon of a surgical robot system according to an embodiment.

FIG. 11 is a perspective view of a surgical instrument according to an embodiment of the present disclosure.

FIGS. 12 and 13 are perspective views of an end tool of the surgical instrument of FIG. 11.

FIGS. 14A to 14B is a plan view of the end tool of the surgical instrument of FIG. 11.

FIGS. 15 and 16 are perspective views of a driving part of the surgical instrument of FIG. 11

FIG. 17 is a plan view of the driving part of the surgical instrument of FIG. 11.

FIG. 18 is a rear view of the driving part of the surgical instrument of FIG. 11.

FIG. 19 is a side view of the driving part of the surgical instrument of FIG. 11.

FIG. 20 is a diagram illustrating the configuration of pulleys and wires of the surgical instrument illustrated in FIG. 11, in detail for the configuration related to a first jaw.

FIG. 21 is a diagram illustrating the configuration of pulleys and wires of the surgical instrument illustrated in FIG. 11, in detail for the configuration related to a second jaw.

FIGS. 22A to 23C are diagrams illustrating a pitch motion of the surgical instrument illustrated in FIG. 11.

FIGS. 24A to 25B are diagrams illustrating a yaw motion of the surgical instrument illustrated in FIG. 11.

FIG. 26 is an exemplary diagram illustrating a coupling relationship among a surgical instrument carriage, an instrument sterile adapter device, and a surgical instrument driving unit according to an aspect of the present disclosure.

FIG. 27 illustrates a coupling relationship between the surgical instrument carriage and the instrument sterile adapter device of FIG. 26 in more detail.

FIG. 28 illustrates a coupling relationship between the surgical instrument carriage and the instrument sterile adapter device of FIG. 27 in another direction.

FIG. 29 is an exemplary horizontal cross-sectional view illustrating the surgical instrument carriage and the instrument sterile adapter device of FIG. 27 in a coupled state.

FIG. 30 illustrates a coupling relationship between the instrument sterile adapter device and the surgical instrument driving unit of FIG. 26 in more detail.

FIG. 31 is an exemplary horizontal cross-sectional view illustrating the instrument sterile adapter device and the surgical instrument driving unit of FIG. 30 in a coupled state.

FIG. 32 is a schematic perspective view of an instrument sterile adapter device having a fixed cover according to an aspect of the present disclosure.

FIG. 33 illustrates the fixed cover of FIG. 32 in a rear view.

FIG. 34 is an exemplary perspective view of the fixed cover of FIG. 32 and the sterile adapter body in a coupled state.

FIG. 35 is an exemplary front view of the fixed cover of FIG. 32 and the sterile adapter body in a coupled state.

FIG. 36 illustrates an exemplary preparation state for coupling to the surgical instrument carriage when the fixed cover of FIG. 32 is coupled to the sterile adapter body.

FIG. 37 illustrates an exemplary coupled state to the surgical instrument carriage when the fixed cover of FIG. 32 is coupled to the sterile adapter body.

FIG. 38 illustrates an exemplary state of the fixed cover being detached from the coupled state of FIG. 37.

FIG. 39 illustrates an exemplary rear view of a fixed cover according to another embodiment of the present disclosure.

### DETAILED DESCRIPTION

Hereinafter, various embodiments of the present disclosure are described in conjunction with the accompanying drawings. Various embodiments of the present disclosure may make various changes and have various embodiments, and specific embodiments are illustrated in the drawings and related detailed descriptions are described. However, this is not intended to limit the various embodiments of the present disclosure to specific embodiments, and should be understood to include all changes and/or equivalents or substitutes included in the spirit and technical scope of the various embodiments of the present disclosure. In connection with the description of the drawings, similar reference numerals have been used for similar components.

Expressions such as “comprise” or “may comprise” that may be used in various embodiments of the present disclosure indicate the presence of the corresponding function, operation, or component disclosed, and do not limit one or more additional functions, operations, or components. In addition, in various embodiments of the present disclosure, terms such as “comprise” or “have” are used to specify the presence of stated features, integers, steps, operations, components, parts, or a combination thereof, but do not preclude the presence or addition of one or more other features, integers, steps, operations, components, parts, or combinations thereof.

In various embodiments of the present disclosure, the expression such as “or” includes any and all combinations of words listed together. For example, “A or B” may include A, B, or both A and B.

Although the expressions such as “first,” “second,” etc. used in various embodiments of the present disclosure may modify various components of the various embodiments, but do not limit the components. For example, the expressions do not limit the order and/or importance of corresponding components. These expressions may be used to distinguish one component from the other components. For example, a first user device and a second user device are both user devices and represent different user devices. For example, a first component may be referred to as a second component without departing from the scope of right of various embodiments of the present disclosure, and similarly, the second component may also be referred to as the first component.

In an embodiment of the present disclosure, terms such as “module,” “unit,” or “part” are used to refer to components that perform at least one function or operation, and these components may be implemented as hardware or software, or as a combination of hardware and software. In addition, a plurality of “modules,” “units,” “parts,” etc. may be integrated into at least one module or chip and implemented with at least one processor, except in the cases where each thereof needs to be implemented with individual specific hardware.

Terms used in various embodiments of the present disclosure are merely used to describe specific embodiments and are not intended to limit the various embodiments of the present disclosure. A singular expression includes a plural expression, unless the context clearly states otherwise.

Unless otherwise defined, all terms including technical and scientific terms used herein have the same meaning as commonly understood by those having ordinary skill in the art to which various embodiments of the present disclosure pertains.

It will be further understood that terms, such as those defined in commonly used dictionaries, should be interpreted as having a meaning that is consistent with their meaning in the context of the relevant art and will not be interpreted in an idealized or overly formal sense unless expressly so defined in various embodiments of the present disclosure.

Hereinafter, various embodiments of the present disclosure will be described in detail using the accompanying drawings.

Laparoscopic surgery refers to a surgery performed by forming a hole in the abdominal cavity of a patient, inserting a narrow and long tube through the hole, and using surgical instruments connected to the end. The surgical instrument may be, for example, an articulated instrument.

In this connection, when a passive surgical instrument is used, the surgical instrument and a control unit operated by a user move symmetrically with respect to a hole in the abdominal cavity, so that more than a certain period of practice is needed until a user becomes familiar with the control. In addition, since the surgical instruments may not be checked with the naked eye, the surgical instruments need to be manipulated while a surgical operator watches the camera images acquired by inserting an endoscopic camera into the abdominal cavity.

This situation is the same even when laparoscopic surgery is performed using a surgical robot system, but there is a benefit of being intuitively controlled compared to manual surgical instruments. As will be described later in the description, the surgical robot system according to an embodiment includes a master robot and a slave robot. The slave robot may be referred to as a surgical robot or surgical instrument, and may refer to a configuration that performs surgery by acting directly on a patient. The master robot may be referred to as a master device or a user input interface, and may refer to a configuration for receiving a user manipulation to control the slave robot.

This type of surgical robot system is mounted with articulated instruments and separates the portion that performs surgery (for example, surgical robot) and the portion that a user manipulates (for example, the master device), and thus intuitive control is possible compared to manual surgical instruments. In other words, the surgical robot system is capable of controlling operations so that surgical instruments may be intuitively controlled by matching the movements of a user with the movements on the laparoscopic camera screen.

However, laparoscopic surgery through surgical robots may inhibit the safety of surgery in certain situations. For example, the surgical robot is driven in response to the remote manipulation signal of a user, so it is impossible to rule out the possibility that the robot arms of the surgical robot physically collide with each other. When a collision of the surgical robot occurs during a surgical process, various issues may occur, such as shaking of surgical instrument, damage to surgical instrument and surgical robots, and damage to tissue.

Accordingly, it is important to prevent collisions of the surgical robot during surgery using a surgical robot system. However, since a user may only remotely manipulate the surgical instrument through the master device and the results of the manipulation may only be checked through images acquired by the endoscopic camera, it is not easy for the user to directly recognize the possibility of collisions of the surgical robot. Accordingly, in order to safely perform surgery using a surgical robot, a technology that may detect collisions in the surgical robot itself is required.

According to embodiments of the present disclosure, a method for preventing a collision between the surgical robots is described. Accordingly, regardless of user intervention, the surgical robots may independently detect collisions between the surgical robots, and based thereon, may stop surgical operations or output guidance to a user regarding whether a collision has occurred so that the surgery may be performed more safely.

### Surgical Robot System Driving

Hereinafter, a method and apparatus for driving a surgical instrument according to embodiments of the present disclosure will be described in more detail with reference to the drawings. Hereinafter, it should be understood that the driving of the surgical robot system in this description includes collision detection of the surgical robot.

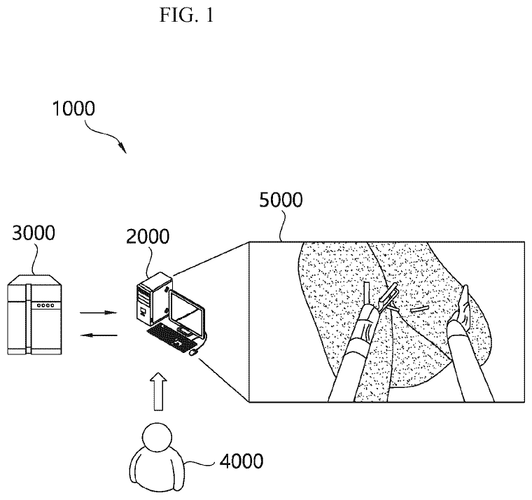

FIG. 1 is a diagram for explaining an example of a system for driving a surgical instrument according to an embodiment.

Referring to FIG. 1, a system 1000 includes a user terminal 2000 and a server 3000. For example, the user terminal 2000 and the server 3000 may be connected to each other through a wired or wireless communication method to transmit and/or receive data to and/or from each other.

For convenience of explanation, although FIG. 1 illustrates that the system 1000 includes the user terminal 2000 and the server 3000, an embodiment of the present disclosure is not limited thereto. For example, other external devices (not shown) may be included in the system 1000, and operations of the user terminal 2000 and the server 3000 to be described below may be implemented by a single device (for example, the user terminal 2000 or the server 3000) or a plurality of devices.

The user terminal 2000 may be a computing apparatus that is provided with a display apparatus and a device (for example, a keyboard, a mouse, or the like) for receiving a user input, and includes a memory and a processor. For example, the display apparatus may be implemented as a touch screen to receive user input. For example, the user terminal 2000 may correspond to a notebook PC, a desktop PC, a laptop, a tablet computer, a smartphone, or the like, but is not limited thereto.

The server 3000 may be an apparatus that communicates with an external device (not shown) including the user terminal 2000. As an example, the server 3000 may be an apparatus that stores various types of data.

Alternatively, the server 3000 may be a computing apparatus including a memory and a processor, and having its own computing capability. For example, the server 3000 may perform at least some of operations of the user terminal 2000 to be described below with reference to the drawings. For example, the server 3000 may also be a cloud server, but is not limited thereto.

According to an aspect, the user terminal 2000 may drive the surgical instrument. In this description, the method for driving the surgical instrument below may be described as being performed by a computing device. The computing device may be, for example, the user terminal 2000 or the server 3000, but is not limited thereto. Any single or plural computing devices including a processor may configure a computing device. Hereinafter, for convenience of explanation, the control procedure of the surgical instrument by the user terminal 2000 may be described, but this is only for explanation, and the method of controlling the surgical instrument according to embodiments of the present disclosure may be performed by any computing device.

Herein, the application of FIG. 1 may be a software program installed for the purpose of activities to drive the surgical robot system of a user 4000. For example, through the application, the user 4000 may generate manipulation information based on the user input to control the surgical robot system.

The user terminal 2000 may output an image 5000 representing the operation of the surgical instrument driven based on the operation of the user 4000. For example, the user terminal 2000 may generate manipulation information based on an amount of change in the reference posture of the user input interface for the user 4000 to control the surgical robot system. Then, the user terminal 2000 may decide the target posture of the surgical instrument corresponding to the manipulation information, and decide the target state information for the driving element. Subsequently, the user terminal 2000 may drive the driving element according to the decided target state information and output the image 5000 representing the operation of the surgical instrument driven in this way. The user 4000 may intuitively understand the operation of the surgical instrument according to the operation of the user through the image 5000 representing the operation of the surgical instrument and manipulate the surgical robot system more accurately.

As described above, at least some of the operations of the user terminal 2000 described below with reference to the drawings may be performed by the server 3000. For example, the server 3000 may perform various activities for controlling the surgical robot system. Alternatively, at least some of these activities may be performed by the server 3000, and at least some thereof may be performed by the user terminal 2000.

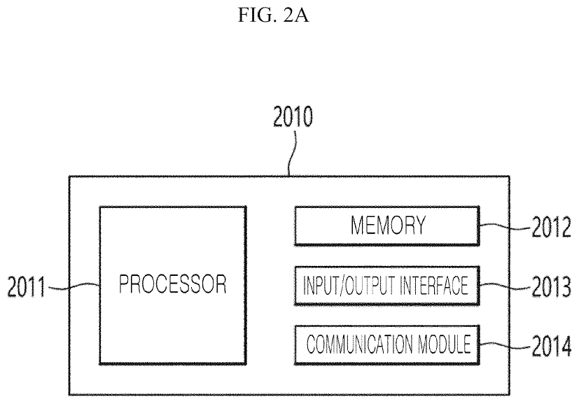

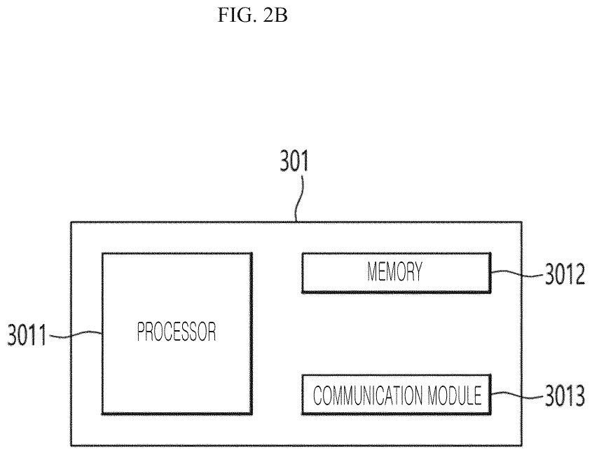

FIG. 2A is a configuration diagram illustrating an example of a user terminal according to an embodiment.

Referring to FIG. 2A, a user terminal 2010 includes a processor 2011, a memory 2012, an input/output interface 2013, and a communication module 2014. For convenience of explanation, FIG. 2A illustrates only components related to an embodiment of the present disclosure. Accordingly, the user terminal 2010 may further include other general-purpose components, in addition to the components illustrated in FIG. 2A. In addition, it is obvious to those skilled in the technical field to which the present disclosure pertains that the processor 2011, the memory 2012, the input/output interface 2013, and the communication module 2014 illustrated in FIG. 2A may also be implemented as independent devices.

The processor 2011 may process commands of a computer program by performing basic arithmetic, logic, and input/output operations. Herein, the commands may be provided from the memory 2012 or an external device (for example, the server 3000, etc.). In addition, the processor 2011 may control the overall operation of other components included in the user terminal 2010.

First, the processor 2011 generates manipulation information regarding the operations of a user to drive the surgical robot system. For example, the processor 2011 may generate manipulation information regarding the operation of the user based on a member that allows the position and function of the surgical instrument to be manipulated by the operation of the user.

The member for manipulating the position and function of the surgical instrument by the operation of a user may be formed in the form of a handle-shaped manipulation member, but is not limited thereto and may be modified and implemented in various shapes to achieve the same purpose. For example, some may be formed in the shape of a handle, and the others may be formed in a different shape, such as a clutch button. In addition, a finger insertion tube may be formed so as to allow the finger(s) of a surgical operator to be inserted therethrough and fixed to facilitate manipulation of a surgical instrument. Hereinafter, in this description, a member that allows manipulation by the operation of the user may also be referred to as the user input interface.

Herein, before the first manipulation of a user of the user input interface, the processor 2011 may update the reference posture of the user input interface with the posture information before manipulation of the user input interface. Since the driving of the surgical instrument by the user may be performed based on the degree to which the user input interface has changed by the user. Hence, by initializing the reference posture of the user input interface to the state before the manipulation before the user performs the first manipulation, the difference between the state of the user input interface after user manipulation and the state of the user input interface before user manipulation, in other words, an amount of change in the user input interface, may be decided.

The processor 2011 may generate manipulation information based on an amount of change in the reference posture of the user input interface. The manipulation information refers to information representing the intuitive operation of a user to manipulate the position and function of the surgical instrument. More specifically, but non-limitingly, the manipulation information may include position information and orientation information on a physical coordinate system of a member that allows a user to manipulate the position and function of the surgical instrument. As an example, the manipulation information may include a transformation matrix representing linear and rotational movement in a homogeneous coordinate system. The transformation matrix may be a homogeneous transformation matrix and may include rotation matrix information and translation vector information. As another example, the manipulation information may include position information and orientation information on a physical coordinate system expressed according to an expression method such as a screw. However, the examples of manipulation information are not limited to the above. The manipulation information may be decided based on an amount of change in the reference posture of the user input interface. Herein, the manipulation information may represent an amount of change with respect to the reference posture, and the reference posture may represent the degree of change of the user input interface with respect to the origin. However, the reference posture and manipulation information may be expressed, for example, by a homogeneous transformation matrix or a screw method as described above.

The processor 2011 may generate manipulation information based on a member that allows a user to manipulate the position and function of the surgical instrument, for example, position information and orientation information of the user input interface. For example, the processor 2011 may generate manipulation information using the difference between the initial position information and initial orientation information of the member that allows the user to manipulate the position and function of the surgical instrument, and the position information and orientation information after the operation of the user of the aforementioned member. According to an aspect, the processor 2011 may generate manipulation information based on an amount of change from the reference posture of the user input interface according to the manipulation of the user.

In addition, based on the manipulation information, the processor 2011 may decide the target posture of the surgical instrument corresponding to the manipulation information. For example, the processor 2011 may decide the target posture of the surgical instrument based on the manipulation information. According to an aspect, the processor 2011 may be configured to decide the target posture based on the correspondence relationship between a predetermined movement of the user input interface and the movement of the surgical instrument.

The processor 2011 may be implemented as an array of a plurality of logic gates, or may be implemented as a combination of a general-purpose microprocessor and a memory in which a program executable in the microprocessor is stored. For example, the processor 2011 may include a general-purpose processor, a central processing unit (CPU), a microprocessor, a digital signal processor (DSP), a controller, a microcontroller, a state machine, and the like. In some circumstances, the processor 110 may include an application-specific integrated circuit (ASIC), a programmable logic device (PLD), a field programmable gate array (FPGA), or the like. For example, the processor 2011 may refer to a combination of processing devices, such as a combination of a digital signal processor (DSP) and a microprocessor, a combination of a plurality of microprocessors, a combination of one or more microprocessors coupled with a digital signal processor (DSP) core, or a combination of any other configurations.

The memory 2012 may include any non-transitory computer-readable recording medium. In an embodiment, the memory 2012 may include a permanent mass storage device such as a random access memory (RAM), a read only memory (ROM), a disk drive, a solid state drive (SSD), a flash memory, etc. In another embodiment, a permanent mass storage device such as a ROM, SSD, a flash memory, a disk drive, etc. may be a separate permanent storage device which is distinguishable from the memory. In addition, an operating system (OS) and at least one program code (for example, a code for the processor 2011 to perform an operation to be described later with reference to the drawings) may be stored in the memory 2012.

These software components may be loaded from a computer-readable recording medium separate from the memory 2012. The separate computer-readable recording medium may be a recording medium that may be directly connected to the user terminal 2010, for example, a computer-readable recording medium, such as a floppy drive, a disk, a tape, a DVD/CD-ROM drive, a memory card, or the like. In addition, the software components may be loaded into the memory 2012 through the communication module 2014 instead of a computer-readable recording medium. For example, at least one program may be loaded into the memory 2012 based on a computer program (for example, a computer program for performing, by the processor 2011, an operation to be described later with reference to the drawings) installed by the files provided through the communication module 2014 by developers or a computer file distribution system that distributes the installation files of applications.

The input/output interface 2013 may be a member for an interface with a device (for example, a keyboard, a mouse, etc.) for input or output, the member being connected to the user terminal 2010 or being included in the user terminal 2010. The input/output interface 2013 may be configured separately from the processor 2011, without being limited thereto, and the input/output interface 2013 may be configured to be included in the processor 2011.

The communication module 2014 may provide a configuration or function for the server 3000 and the user terminal 2010 to communicate with each other through a network. In addition, the communication module 2014 may provide a configuration or function for the user terminal 2010 to communicate with another external device. For example, a control signal, a command, data, etc. provided according to the control of the processor 2011, may be transmitted to the server 3000 and/or an external device through the communication module 2014 and the network.

Although not illustrated in FIG. 2A, the user terminal 2010 may further include a display apparatus. For example, the display apparatus may be implemented as a touch screen. Alternatively, the user terminal 2010 may be connected to an independent display apparatus through a wired or wireless communication method to transmit and/or receive data to and/or from each other. For example, a video or image of driving the surgical instrument using driving information may be provided through the display apparatus.

FIG. 2B is a configuration diagram illustrating an example of a server according to an embodiment.

Referring to FIG. 2B, the server 3010 includes a processor 3011, a memory 3012, and a communication module 3013. For convenience of explanation, FIG. 2B illustrates only components related to an embodiment of the present disclosure. Accordingly, the server 3010 may further include other general-purpose components, in addition to the components illustrated in FIG. 2B. In addition, it is obvious to those skilled in the technical field to which the present disclosure pertains that the processor 3011, the memory 3012, and the communication module 3013 illustrated in FIG. 2B may also be implemented as independent devices.

The processor 3011 may perform various activities for controlling the surgical robot system. In other words, at least one of the operations of the processor 2011 described above with reference to FIG. 2A may be performed by the processor 3011. In this connection, the user terminal 2010 may output information transmitted from the server 3010 through the display apparatus.

Since the implementation example of the processor 3011 is the same as the implementation example of the processor 2011 described above with reference to FIG. 2A, the detailed description thereof is omitted.

The memory 3012 may store various pieces of data, such as data necessary for the operation of the processor 3011 and data generated according to the operation of the processor 3011. Additionally, an operating system (OS) and at least one program (for example, a program necessary for the processor 3011 to operate, etc.) may be stored in the memory 3012.

Since the implementation example of the memory 3012 is the same as the implementation example of the memory 2012 described above with reference to FIG. 2A, the detailed description thereof will be omitted.

The communication module 3013 may provide a configuration or function for the server 3010 and the user terminal 2010 to communicate with each other through a network. Additionally, the communication module 2014 may provide a configuration or function for the server 3010 to communicate with other external devices. For example, control signals, commands, data, etc. provided under control of the processor 3011 may be transmitted to the user terminal 2010 and/or an external device through the communication module 3013 and a network.

### Surgical Robot System Configuration

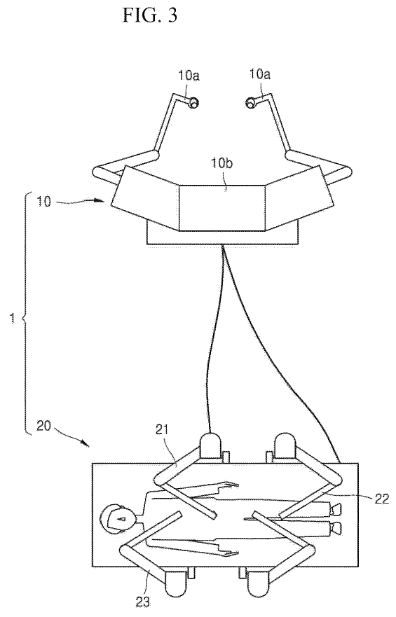

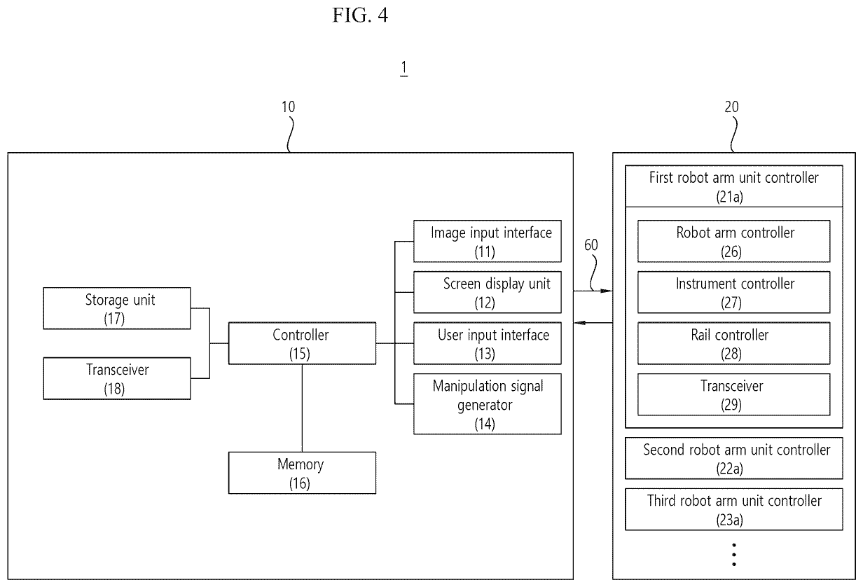

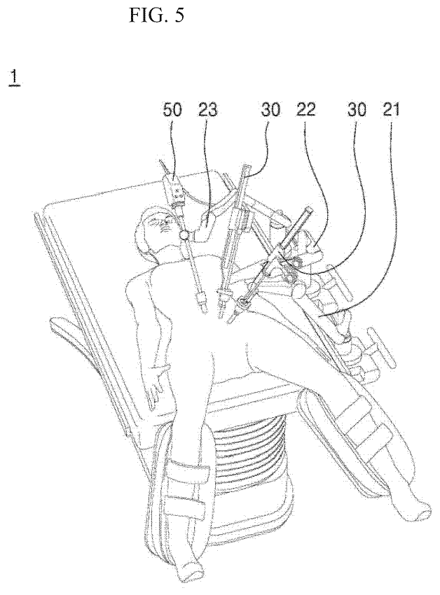

FIG. 3 is a diagram illustrating a surgical robot system according to an embodiment. FIG. 4 is a block diagram illustrating the internal configuration of the surgical robot system of FIG. 3. FIG. 5 is a perspective view of a slave robot of the surgical robot system of FIG. 3 and a surgical instrument mounted thereon.

Referring to FIGS. 3 to 5, a surgical robot system 1 includes a master robot 10, a slave robot 20, a surgical instrument 30 and a laparoscope camera 50.

The master robot 10 includes manipulation members 10a and a display member 10b, and the slave robot 20 includes one or more robot arm units 21, 22, and 23.

As a non-limiting example, the master robot 10 may include the manipulation members 10a so that a surgical operator may grip and manipulate the same respectively with both hands. The manipulation members 10a may be implemented as two or more handles as illustrated in FIG. 3, and manipulation signals according to the handle manipulation of the surgical operator are transmitted to the slave robot 20 through a wired or wireless communication network so that the robot arm units 21, 22, and 23 are controlled. In other words, surgical operations such as positioning, rotation, and cutting work of the robot arm units 21, 22, and 23 may be performed by the handle manipulation of the surgical operator. Herein, the manipulation signal may be, for example, manipulation information generated by a processor, but is not limited thereto.

For example, the surgical operator may manipulate the robot arm units 21, 22, and 23 using manipulation levers in the form of a handle. The manipulation lever as described above may have various mechanical configurations according to the manipulation method thereof, and may be provided in various configurations for operating the robot arm units 21, 22, and 23 of the slave robot 20 and/or other surgical equipment, such as a master handle manipulating the operation of each of the robot arm units 21, 22, and 23 and various input tools added to the master robot 10 for manipulating the functions of the entire system such as joystick, keypad, trackball, foot pedal, and touch screen. Herein, the manipulation member 10a is not limited to the shape of a handle and may be applied without any limitation as long as the manipulation member 10a may control operations of the robot arm units 21, 22, and 23 through a network such as a wired or wireless communication network.

According to an embodiment of the present disclosure, manipulation information may be generated based on the manipulation lever or manipulation member 10a described above. For example, according to an embodiment of the present disclosure, manipulation information may be generated based on the operation of a user manipulating the manipulation lever or manipulation member 10a. However, examples of generating manipulation information are not limited to the above description.

Alternatively, a voice input or a motion input may also be applied as user input. In other words, a user may wear, on the head thereof, glasses or a head mount display (HMD), to which a sensor is attached, and a laparoscope camera 50 may move according to a direction of the gaze. Alternatively, when the user issues a command with voice, such as “left”, “right”, “first arm”, “second arm”, and the like, the voice command may be recognized and the motion may be performed. For example, an embodiment of the present disclosure may generate manipulation information based on the voice of the user.

An image captured through the laparoscope camera 50 is displayed as a screen image on the display member 10b of the master robot 10. For example, the image captured via the laparoscope camera 50 may include a surgical site of a patient, a surgical instrument being inserted into the surgical side of a patient, a motion of the surgical instrument, and the like. For example, the display member 10b may display a video image corresponding to the motion of the surgical instrument being inserted into the surgical site of the patient. In addition, a predetermined fictive manipulation plate may be displayed independently or displayed together with the image captured by the laparoscope camera 50 on the display member 10b. The arrangement, configuration, and the like of such a fictive manipulation plate will not be described in detail.

The display member 10b may include one or more monitors, each of which may individually display information necessary for surgery. The quantity of monitors may be variously decided depending on the type or kind of information that needs to be displayed.

One or more slave robots 20 may be provided to operate a patient. As a non-limiting example, the surgical robot system 1 may include a slave robot 20 (which may be referred to as the “first robot”) coupled with a surgical instrument 30 (which may be referred to as the “first robot”) and a slave robot 20 (which may be referred to as the “second robot”) coupled with a laparoscope camera 50 (which may be referred to as the “second robot”), respectively. In other words, the laparoscope camera 50 for allowing a surgical site or a surgical instrument to be displayed as a screen image through the display member 10b may be implemented as a separate slave robot 20 independent of the slave robot 20 to which the surgical instrument 30 is coupled. It should also be understood that, as described above, the embodiments of the present disclosure may be used universally for surgeries in which various surgical endoscopes other than laparoscopes (for example, thoracoscopic, arthroscopic, rhinoscopic, and the like) are used.

In one example, two of the robot arm units 21, 22, and 23 may have the surgical instrument 30 attached thereto, and one of the robot arm units 21, 22, and 23 may have the laparoscope camera 50 attached thereto. In addition, a surgical operator may select the slave robot 20 (or the robot arm unit 21, 22, or 23) to be controlled via the master robot 10. As described above, by directly controlling a total of three or more surgical instruments through the master robot 10, the surgical operator may accurately and freely control various instruments according to the intention of the surgical operator without a surgical assistant.

As another example, the slave robot 20 may include one or more robot arm units 21, 22, and 23. Although FIGS. 3 to 5 exemplarily show one robot arm unit 21, 22, 23 coupled to one slave robot 20, it is noted that the technical spirit of the present disclosure is not limited to this. For example, two robot arm units may be coupled to one slave robot 20, with a surgical instrument 30 attached to one of the robot arm units and a laparoscope camera 50 attached to the other robot arm unit. However, even when a plurality of robot arm unit are coupled to a single slave robot 20, each of the robot arm units 21, 22, and 23 may be provided in the form of a module that may operate independently of each other, and in this connection, an algorithm for preventing a collision between the robot arm units 21, 22, and 23 may be applied to the surgical robot system 1.

The slave robot 20 may include one or more robot arm units 21, 22, and 23. Herein, each of the robot arm units 21, 22, and 23 may be provided in the form of a module that may operate independently of each other, and in this connection, an algorithm for preventing a collision between the robot arm units 21, 22, and 23 may be applied to the surgical robot system 1.

In general, a robot arm refers to an apparatus having a function similar to that of the arm and/or the wrist of a human being and having a wrist portion to which a predetermined tool may be attached. In an embodiment of the present disclosure, the robot arm units 21, 22, and 23 may each be defined as a concept encompassing all of the components such as an upper arm, a lower arm, a wrist, and an elbow, a surgical instrument (or a laparoscope camera) coupled to the wrist portion, and the like. Alternatively, the robot arm unit may also be defined as a concept that includes only components for driving the surgical instrument (or a laparoscope camera), excluding the surgical instrument (or a laparoscope camera) coupled to the wrist portion.

The robot arm units 21, 22, and 23 of the slave robot 20 described above may be implemented to be driven with multiple degrees of freedom. The robot arm units 21, 22, and 23 may include, for example, a surgical instrument (or a laparoscope) inserted into a surgical site of a patient, a yaw driving unit for rotating the surgical instrument in a yaw direction according to a surgical position, a pitch driving unit for rotating the surgical instrument in a pitch direction perpendicular to a rotational driving of the yaw driving unit, a transfer driving unit for moving the surgical instrument in a length direction, a rotation driving unit for rotating the surgical instrument, and a surgical instrument driving unit for incising or cutting the surgical lesion by driving an end effector at an end of the surgical instrument. However, the configuration of the robot arm units 21, 22, and 23 is not limited thereto, and it should be understood that this example does not limit the scope of the present disclosure. Herein, a detailed description of the actual control process, such as rotation and movement of the robot arm units 21, 22, and 23 in a corresponding direction by the surgical operator manipulating the manipulating member 10a, will be omitted.

The master robot 10 may perform various activities such as at least one of generating manipulation information based on an amount of change in the reference posture of the user input interface for controlling the surgical instrument, deciding the target posture of the surgical instrument corresponding to the manipulation information, deciding the target state information for the driving element, or driving the driving element according to the target state information.

For example, the master robot 10 transmits at least one piece of the manipulation information or the target state information of the driving element determined based thereon to the slave robot 20 through a wired or wireless communication network to control the robot arm units 21, 22, and 23. In other words, surgical operations such as positioning, rotation, and cutting work of the robot arm units 21, 22, and 23 may be performed by the handle manipulation of a surgical operator. In other words, when the manipulation information is decided by the master robot 10, the decided manipulation information may be transmitted to the slave robot 20 through a wired or wireless communication network, and the slave robot 20 may decide the target state information based on the manipulation information. According to another aspect, the master robot 10 may decide manipulation information, decide target state information corresponding thereto, and transmit the decided target state information to the slave robot 20.

Referring to FIG. 4, in an embodiment of the present disclosure, the master robot 10 may include an image input interface 11, a screen display unit 12, a user input interface 13, a manipulation signal generator 14, a controller 15, a memory 16, a storage unit 17, and a transceiver 18.

At least some of the configurations of the master robot 10 may be included in the user terminal of FIG. 2A. For example, the manipulation signal generator 14 and the controller 15 may be included in the processor 2011, the memory 16 and the storage unit 17 may be included in the memory 2012, and the transceiver 18 may be included in the communication module 2014, but the example of the master robot 10 is not limited to the above.

The image input interface 11 may receive an image captured by a camera provided in the laparoscope camera 50 of the slave robot 20 through a wired or wireless communication network. For example, the images captured through the laparoscope camera 50 may include images of a surgical site of a patient, surgical instruments being inserted into the surgical site of the patient, the motion of the surgical instruments, and the like. Further, such images may include an image representing the operation of the surgical instrument driven according to target state information.

The screen display unit 12 outputs a screen image corresponding to the image received through the image input interface 11 as visual information. In addition, the screen display unit 12 may further output information corresponding to biometric information of a subject to be treated, when the biometric information is input. In addition, the screen display unit 12 may further output image data (for example, an X-ray image, a CT image, an MRI image, or the like) associated with a patient for a surgical site. Herein, the screen display unit 12 may be implemented in the form of a display member (see 10b of FIG. 3), and an image processing process for allowing the received image to be output as a screen image through the screen display unit 12 may be performed by the controller 15. Herein, the image may include an image representing the operation of the surgical instrument driven according to target state information.

In the embodiment illustrated in FIG. 4, the image input interface and the screen display unit are illustrated as being included in the master robot 10, but an embodiment of the present disclosure is not limited thereto. The display member may be provided as a separate member spaced apart from the master robot 10. Alternatively, the display member may be provided as one component of the master robot 10. In addition, in another embodiment, a plurality of display members may be provided, one of which may be disposed adjacent to the master robot 10, and others thereof may be disposed at some distance from the master robot 10.

Herein, the screen display unit 12 (in other words, the display member 10b of FIG. 3) may be provided as a three-dimensional display apparatus. In detail, the three-dimensional display apparatus refers to an image display apparatus in which depth information is added to a two-dimensional image by applying a stereoscopic technique, and this depth information is used to enable an observer to feel a three-dimensional living feeling and a sense of reality. The surgical robot system 1 according to an embodiment of the present disclosure may provide a more realistic fictive environment to a user by including a three-dimensional display apparatus as the screen display unit 12.

The user input interface 13 is a member for allowing a surgical operator to manipulate the positions and functions of the robot arm units 21, 22, and 23 of the slave robot 20. The user input interface 13 may be formed in the form of a handle-shaped manipulation member (see 10a of FIG. 3) as illustrated in FIG. 3, but the shape thereof is not limited thereto and may be implemented by being modified in various shapes to achieve the same purpose. In addition, for example, some of the user input interface 13 may be formed in the shape of a handle, and the others thereof may be formed in a different shape, such as a clutch button. In addition, a finger insertion tube or insertion ring may be further formed so as to allow the fingers of a surgical operator to be inserted therethrough and fixed to facilitate manipulation of a surgical instrument.

According to an embodiment of the present disclosure, manipulation information may be generated based on the operation of a surgical operator on the user input interface 13. For example, according to an embodiment of the present disclosure, manipulation information can be generated based on the operation of the surgical operator manipulating the user input interface 13. However, examples of generating manipulation information are not limited to the above.

The manipulation signal generator 14 generates a corresponding manipulation signal when a surgical operator manipulates the user input interface 13 to move the position of the robot arm units 21, 22, and 23 or manipulate the surgical operation. As an example, the manipulation signal generator 14 may generate corresponding manipulation information when the surgical operator manipulates the user input interface 13 to move the position of the robot arm units 21, 22, and 23 or manipulate the surgical operation.

For example, the manipulation signal generator 14 transmits the generated manipulation signal to the controller 15 or to the slave robot 20 through the transceiver 18. The manipulation signal may be transmitted and received through a wired or wireless communication network. Based on the transmitted manipulation signal, the controller 15 may control the slave robot 20, the surgical instrument 30, or the laparoscope camera 50 to operate. Alternatively, based on the transmitted manipulation signal, a robot arm controller 26 included in the slave robot 20 may control the robot arm units 21, 22, and 23 to operate. Alternatively, based on the transmitted manipulation signal, an instrument controller 27 included in the slave robot 20 may control the surgical instrument 30 or laparoscope camera 50 to operate. However, the method by which the operation of the slave robot 20, the surgical instrument 30, or the laparoscope camera 50 is controlled based on the manipulation signal is not limited to the aforementioned method.

The instrument controller 27 receives the manipulation signal generated by the manipulation signal generator 14 of the master robot 10 and controls the surgical instrument 30 to operate according to the manipulation signal.

The controller 15 is a kind of central processing device, and controls the operation of each component so that the aforementioned functions may be performed. In an example, the controller 15 may perform a function of transforming an image input through the image input interface 11 into a screen image to be displayed through the screen display unit 12. As another example, the controller 15 may generate the target posture of the robot arm units 21, 22, and 23 based on manipulation information. In addition, the controller 15 may decide target state information of the at least one driving element based on the target posture. In addition, the controller 15 may drive the robot arm units 21, 22, and 23 based on the decided target state information.

According to the above description, it has been described that the controller 15 calculates the target posture based on the manipulation information and target state information, which may be performed by other controllers according to an embodiment of the present disclosure (for example, by the robot arm controller 26, or the instrument controller 27), without being limited thereto.

The memory 16 may perform a function of temporarily or permanently storing data processed by the controller 15. Herein, the memory 16 may include a magnetic storage medium or a flash storage medium, but the scope of the present disclosure is not limited thereto.

The storage unit 17 may store data received from the slave robot 20. In addition, the storage unit 17 may store various pieces of input data (for example, patient data, device data, surgery data, and the like).

The transceiver 18 interworks with a communication network 60 to provide a communication interface necessary for transmitting and receiving image data transmitted from the slave robot 20 and control data transmitted from the master robot 10. The image data transmitted from the slave robot 20 may include an image representing the operation of the surgical instrument driven according to target state information. The control data transmitted from the master robot 10 may include at least one piece of manipulation information on an amount of change in the user input interface or target state information on an operation of the slave robot 20.

The slave robot 20 includes a plurality of robot arm unit controllers 21a, 22a, and 23a. In addition, the robot arm unit controller 21a includes a robot arm controller 26, an instrument controller 27, and a transceiver 29. Further, the robot arm unit controllers 21a may further include a rail controller 28.

Referring to FIGS. 4 and 5, the rail controller 28 may control the path of movement of the surgical instrument 30 on the robot arm units 21, 22, 23 to enable movement along a preset path, specifically along the longitudinal direction of the connection part 310 described later herein.

The robot arm controller 26 may receive a manipulation signal generated by the manipulation signal generator 14 of the master robot 10, and may serve to control the robot arm units 21, 22, and 23 to operate according to the manipulation signal. For example, the robot arm controller 26 may receive manipulation information or target state information calculated from the master robot 10, and may serve to control the robot arm units 21, 22, and 23 to operate accordingly.

The instrument controller 27 may receive a manipulation signal generated by the manipulation signal generator 14 of the master robot 10, and may serve to control the surgical instrument 30 to operate according to the manipulation signal. For example, the instrument controller 26 may receive manipulation information or target state information calculated from the master robot 10, and may serve to control the surgical instrument 30 to operate accordingly.

The transceiver 29 interworks with the communication network 60 to provide a communication interface necessary for transmitting and receiving image data transmitted from the slave robot 20 and control data transmitted from the master robot 10. The image data transmitted from the slave robot 20 may include an image representing the operation of the surgical instrument driven according to target state information. The control data transmitted from the master robot 10 may include at least one piece of manipulation information on an operation of the slave robot 20 or target state information.

The communication network 60 serves to connect the master robot 10 and the slave robot 20. In other words, the communication network 60 refers to a communication network for providing an access path so that data may be transmitted and received between the master robot 10 and the slave robot 20 after the master robot 10 and the slave robot 20 are connected. The communication network 60 may be, for example, a wired network such as local area networks (LANs), wired area networks (WANs), metropolitan area networks (MANs), and integrated service digital networks (ISDNs), or a wireless network such as wireless LANs, code division multiple access (CDMA), Bluetooth, and satellite communication, but the scope of an embodiment of the present disclosure is not limited thereto.

### Modular Slave Robot

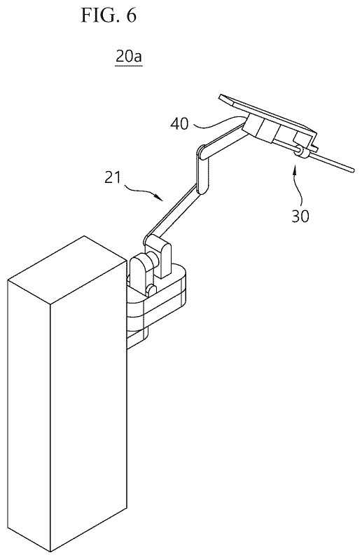

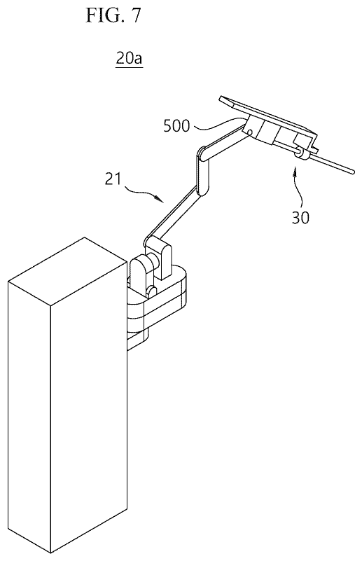

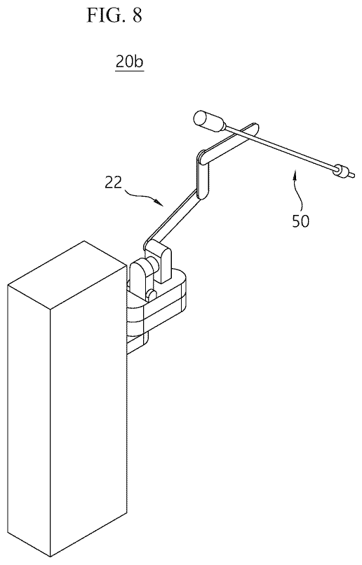

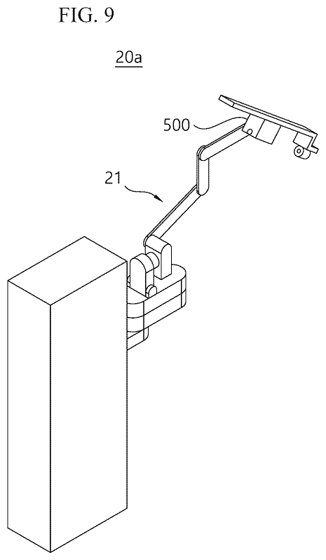

FIG. 6 is a perspective view of a modular slave robot and a surgical instrument mounted thereon according to an aspect of the surgical robot system of FIG. 3. FIG. 7 is a diagram illustrating a state in which the instrument case is removed from FIG. 6. FIG. 8 is a perspective view of a modular slave robot and a laparoscopic surgical camera mounted thereon according to another aspect of the surgical robot system of FIG. 3. FIG. 9 is a diagram illustrating a state in which the surgical instrument is removed from the slave robot of FIG. 6.

The surgical instrument 30 or the laparoscope camera 50, which will be described below, may be connected to and installed in the robot arm unit 21, 22, or 23. Referring to FIG. 6, an instrument case 40 may cover the surgical instrument 30, and may be connected to the robot arm unit 21. The instrument case 40 may cover one side of the surgical instrument 30 exposed to the outside, so as to prevent external foreign substances from reaching the surgical instrument 30, and protect the surgical instrument 30 from being damaged due to external shock.

Referring to FIG. 7, the surgical instrument 30 may be connected to and installed in the robot arm unit 21 of a modular slave robot 20a according to an embodiment. In an embodiment of the present disclosure, the modular slave robot 20a in which the surgical instrument 30 is installed in the robot arm unit 21 may be referred to as a “surgical robot.” Referring to FIG. 8, the laparoscope camera 50 may be connected to and installed in the robot arm unit 22 of the modular slave robot 20b according to an embodiment. In an embodiment of the present disclosure, the modular slave robot 20b in which the laparoscope camera 50 is installed in the robot arm unit 22 may be referred to as a “camera robot.”

Referring to FIGS. 6 to 9, only one robot arm unit 21, 22 among the robot arm units 21, 22, 23 is exemplarily illustrated in a form in which one slave robot 20a or 20b is coupled with the surgical instrument 30 or the laparoscope camera 50, but the technical idea of the present disclosure is not limited thereto. As described above, two of the robot arm units 21, 22, 23 may be attached to the surgical instrument 30, one may be attached to the laparoscope camera 50, and two or more robot arm units may be provided for one slave robot.

Referring to FIGS. 6 to 9, a motor pack 500 is connectable to the surgical instrument 30, and may be coupled to the surgical robot 20a, specifically, the robot arm unit 21, and fixed in position.

The instrument case 40 is connected to one side of the surgical instrument 30, and the motor pack 500 is connected and coupled to the other side opposite thereto. The motor pack 500 receives power source from the outside to generate power, and may transmit the power generated from the motor pack 500 to the surgical instrument 30, thereby allowing the surgical instrument 30 to perform pitch motion, yaw motion, actuation motion, and roll motion.

### Active/Passive Arm Unit

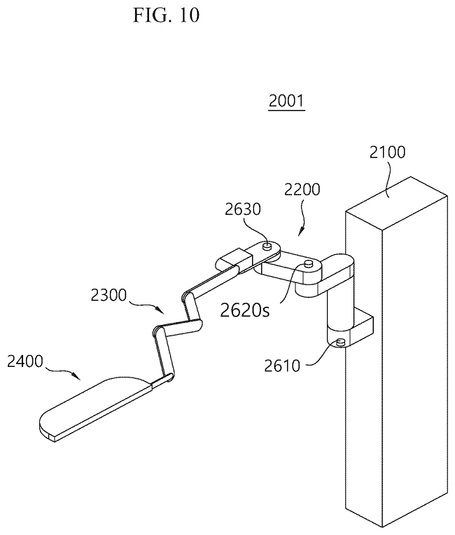

FIG. 10 is a perspective view of another example of a modular slave robot and a surgical instrument mounted thereon of a surgical robot system according to an embodiment.

Referring to FIG. 10, a surgical robot 2001 according to an embodiment may include a body 2100, an active arm unit 2300, and a surgical instrument 2400. In addition, the surgical robot 2001 according to another embodiment may further include a passive arm unit 2200 and one or more angle measuring sensors 2610, 2620s, 2630.

The body 2100 may refer to a main body connected to the robot arm unit. For example, the robot arm unit and the body 2100 may configure one independent slave robot 20. In addition, the body 2100 may include a moving member (not shown) that allows the surgical robot 2001 to be disposed at a desired position in an operating room. For example, the body 2100 may be provided with wheels so as to move freely. The body 2100 may further include a fixing member (not shown) that allows the surgical robot 2001 to be fixed to the operating room and prevented from moving. For example, after the disposition of the surgical robot 2001 is completed and an surgical operator begins surgery, the fixing member may fix the body 2100 to a predetermined position in the operating room so that the surgical robot 2001 may not move for the sake of the stability of the surgery.

The robot arm unit included in the surgical robot 2001 may include at least one of a passive arm unit 2200 or an active arm unit 2300. For example, the surgical robot 2001 may be configured of the body 2100 and the active arm unit 2300, or may be configured of the body 2100, the passive arm unit 2200, and the active arm unit 2300. For example, when the robot arm unit of the surgical robot 2001 is configured only of the active arm unit 2300, the active arm unit 2300 may be directly connected to the body 2100. As another example, when the robot arm unit of the surgical robot 2001 is configured of the passive arm unit 2200 and the active arm unit 2300, the body 2100 may be directly connected to the passive arm unit 2200, and the passive arm unit 2200 may be connected at one end to the body 2100 and at the other end to the active arm unit 2300.

The passive arm unit 2200 may be defined as a robot arm whose position, direction, angle, or the like are manipulated by external force. For example, an surgical operator or a surgical assistant assisting the surgical operator may manipulate the movement of the passive arm unit 2200 by applying physical force. In addition, the position, direction, angle, or the like of the passive arm unit 2200 may be maintained when there is no external force manipulating the movement. In other words, when the aforementioned surgical operator or surgical assistant manipulates the position, direction, angle, or the like before the surgery begins, the position, direction, angle, or the like of the passive arm unit 2200 may be maintained without change during the surgery. From this perspective, the body 2100 may be included in the passive arm unit 2200 in that the position to which the surgical operator or surgical assistant moves the body 2100 before the surgery begins may be maintained without change during the surgery.

The passive arm unit 2200 may include an angle measurement sensor 2610, 2620s, 2630. Herein, the angle measurement sensor 2610, 2620s, 2630 may refer to a sensor that monitors the movement of the passive arm unit 2200. For example, the angle measurement sensor 2610, 2620s, 2630 may measure or calculate the position, direction, angle, etc. of the passive arm unit 2200. For example, the angle measurement sensor 2610, 2620s, 2630 may be implemented as a sensor capable of measuring the change amount in position, speed, and direction of an object, such as a rotary encoder, a linear encoder, or a potentiometer.

In addition, the angle measurement sensor 2610, 2620s, 2630 may be installed so as to be positioned between any two passive arm units. For example, the number of angle measurement sensors included in the surgical robot 2001 may be one less than the number of the passive arm units 2200. Referring to FIG. 10, the passive arm unit 2200 connecting the body 2100 and the active arm unit 2300 may include a total of four robot arms, and the surgical robot 2001 according to an embodiment may include a total of three angle measurement sensors.

The active arm unit 2300 may be defined as a robot arm in which the position, direction, angle, or the like of the robot arm are automatically manipulated through an internal control algorithm. For example, when a surgical operator manipulates the user input interface 13 to manipulate the active arm unit 2300, the manipulation signal generator 14 may generate a manipulation signal corresponding to the motion of the surgical operator manipulating the user input interface 13 and transmit the same to the robot arm controller of the active arm unit 2300.

Thereafter, the robot arm controller of the active arm unit 2300 may control the active arm unit 2300 to move in position, rotate, or the like according to the control algorithm based on the received control signal. In other words, the position, direction, angle, or the like of the active arm unit 2300 may be manipulated when there is manipulation by the surgical operator, regardless of before or after the start of surgery. Since the active arm unit 2300 is manipulated through a control algorithm rather than external force, an external energy supply through a motor or actuator is needed. Accordingly, the active arm unit 2300 may include one or more motors or actuators.

The surgical instrument 2400 included in the surgical robot 2001 may be connected to at least one of the passive arm unit 2200 and the active arm unit 2300. FIG. 10 illustrates the surgical robot 2001 to which a surgical instrument 2400 is coupled, but is not limited thereto. In other words, the contents described with reference to FIG. 10 may be equally applied to a camera robot to which a laparoscopic surgical camera (not shown) is coupled.

### Surgical Instrument

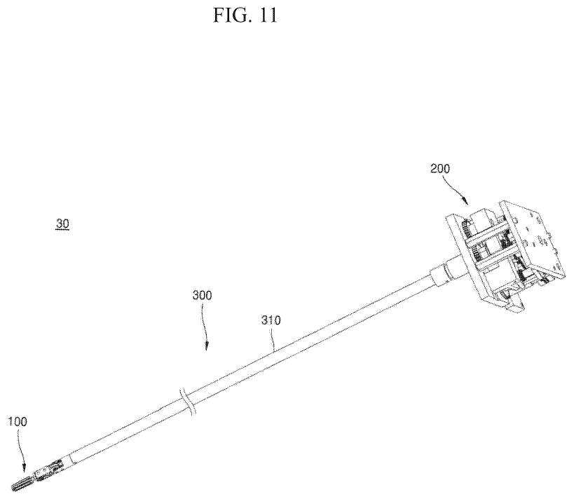

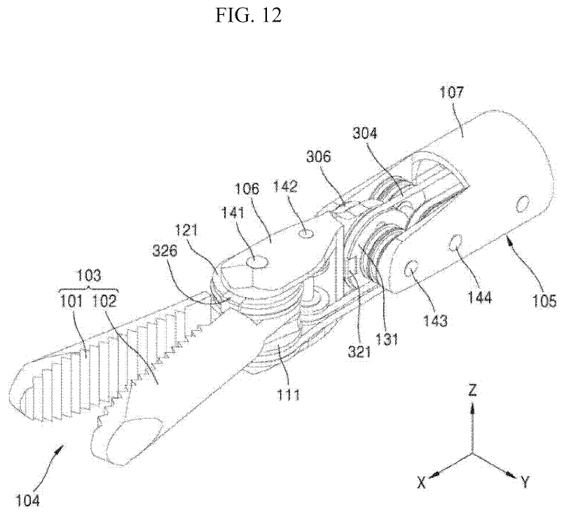

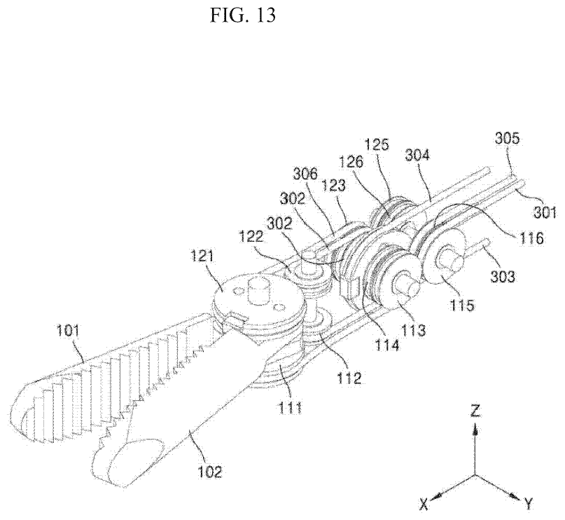

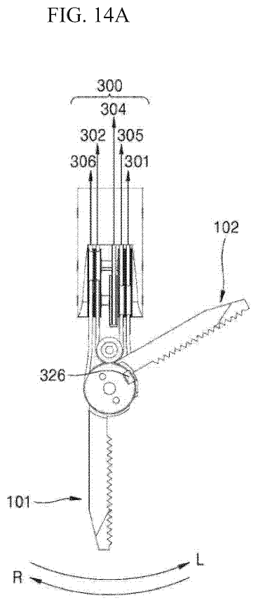

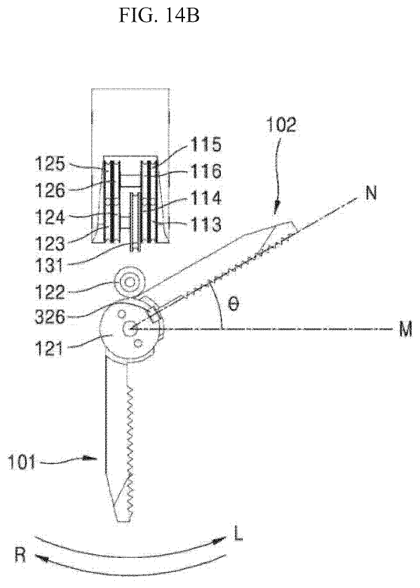

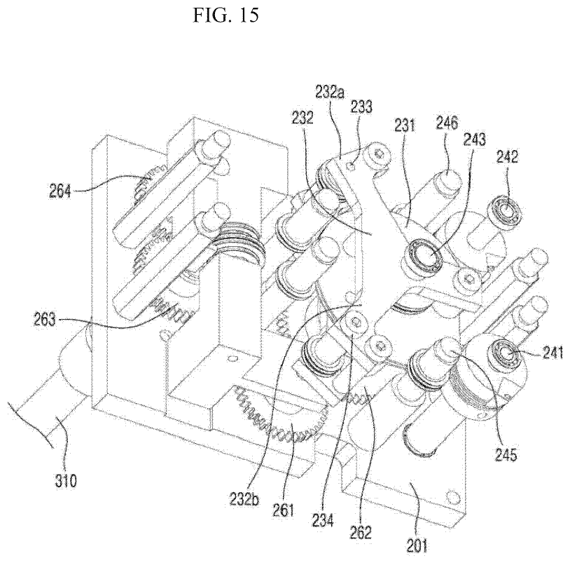

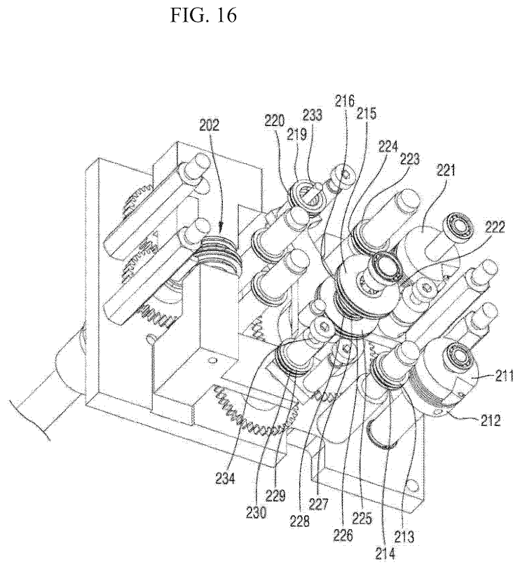

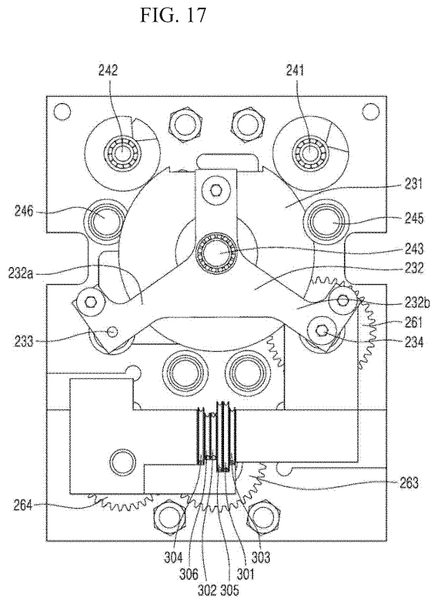

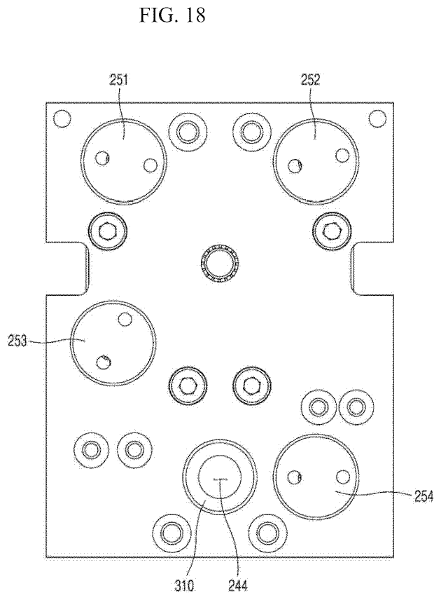

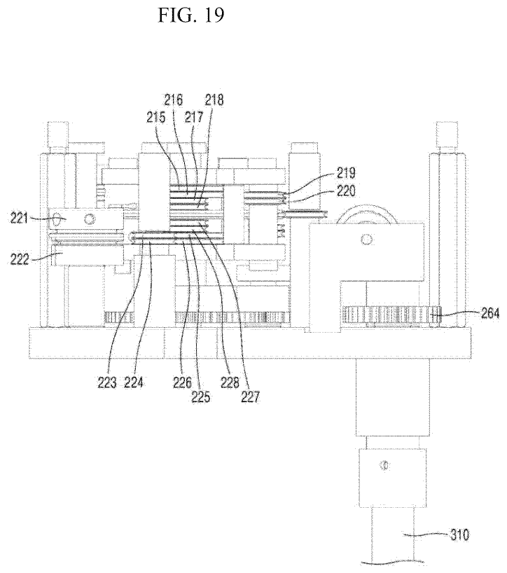

FIG. 11 is a perspective view of a surgical instrument according to an embodiment of the present disclosure, FIGS. 12 and 13 are perspective views of an end tool of the surgical instrument of FIG. 11, and FIGS. 14A to 14B is a plan view of the end tool of the surgical instrument of FIG. 11. FIGS. 15 and 16 are perspective views of a driving part of the surgical instrument of FIG. 11, FIG. 17 is a plan view of the driving part of the surgical instrument of FIG. 11, FIG. 18 is a rear view of the driving part of the surgical instrument of FIG. 11, and FIG. 19 is a side view of the driving part of the surgical instrument of FIG. 11.

Referring first to FIG. 11, the surgical instrument 30 according to an embodiment of the present disclosure may include an end tool 100, a driving part 200, and a power transmission part 300, and the power transmission part 300 may include a connection part 310.

The connection part 310 is formed in the shape of a hollow shaft, in which one or more wires (to be described later) may be accommodated, and may have one end portion to which the driving part 200 is coupled and the other end portion to which the end tool 100 is coupled, and serve to connect the driving part 200 and the end tool 100.

The driving part 200 is formed at one end portion of the connection part 310 and provides an interface capable of being coupled to the robot arm unit (see 21 or the like in FIG. 3). Accordingly, when a user operates the master robot (see 10 in FIG. 3), a motor (not shown) of the robot arm unit (see 21 or the like in FIG. 3) is operated so that the end tool 100 of the surgical instrument 30 can perform a motion corresponding thereto, and a driving force of the motor (not shown) is transmitted to the end tool 100 through the driving part 200. In other words, it may be described that the driving part 200 itself becomes an interface that connects between the surgical instrument 30 and the slave robot 20.

For example, when the user input part 13 (see FIG. 3) is operated by a user, a motor (not shown) of the robot arm unit 21 or the like (see FIG. 3) operates so that the end tool 100 of the surgical instrument 30 can perform a motion corresponding thereto, and a driving force of the motor (not shown) may be transmitted to the end tool 100 through the driving part 200.

The end tool 100 is formed on the other end portion of the connection part 310, and performs necessary motions for surgery by being inserted into a surgical site. In an example of the above-described end tool 100, as shown in FIG. 12, a pair of jaws 101 and 102 for performing a grip motion may be used. However, the embodiment of the present disclosure is not limited thereto, and various devices for performing surgery may be used as the end tool 100. For example, a configuration such as a cantilever cautery may also be used as the end tool. The above-described end tool 100 is connected to the driving part 200 by the power transmission part 300 and receives a driving force through the power transmission part 300 to perform a motion necessary for surgery, such as a gripping motion, a cutting motion, a suturing motion, or the like.

Here, the end tool 100 of the surgical instrument 30 according to an embodiment of the present disclosure is formed to be rotatable in at least two or more directions, for example, the end tool 100 may be formed to perform a pitch motion around a rotation shaft 143 of FIG. 12 and simultaneously perform a yaw motion and an actuation motion around a rotation shaft 141 of FIG. 12.

Here, each of a pitch motion, a yaw motion, an actuation motion, and a roll motion as used in the present disclosure are defined as follows.

First, the pitch motion means a motion of the end tool 100 rotating in a vertical direction with respect to an extension direction of the connection part 310 (an X-axis direction of FIG. 11), that is, a motion rotating around the Y-axis of FIG. 11. In other words, the pitch motion means a motion of the end tool 100, which is formed to extend from the connection part 310 in the extension direction of the connection part 310 (the X-axis direction of FIG. 11), rotating vertically around the Y-axis with respect to the connection part 310.

Next, the yaw motion means a motion of the end tool 100 rotating in left and right directions, that is, a motion rotating around a Z-axis of FIG. 11, with respect to the extension direction of the connection part 310 (the X-axis direction of FIG. 11). In other words, the yaw motion means a motion of the end tool 100, which is formed to extend from the connection part 310 in the extension direction of the connection part 310 (the X-axis direction of FIG. 11), rotating horizontally around the Z-axis with respect to the connection part 310. That is, the yaw motion relates to a motion of two jaws 101 and 102, which are formed on the end tool 100, rotating around the Z-axis in the same direction.

Meanwhile, the actuation motion means a motion of the end tool 100 rotating around the same shaft of rotation as that of the yaw motion, while the two jaws 101 and 102 rotate in the opposite directions so as to be closed or opened. That is, the actuation motion means rotating motions of the two jaws 101 and 102, which are formed on the end tool 100, in the opposite directions around the Z-axis.

Defining this from another perspective, the yaw rotation may be defined as a motion in which an end tool jaw pulley (to be described later) rotates around the rotation shaft 141, which is an end tool jaw pulley rotation shaft, and the pitch rotation may be defined as a motion in which the end tool jaw pulley revolves around the rotation shaft 143, which is an end tool pitch rotation shaft.

The roll motion refers to a motion in which the surgical instrument rotates with the connection part 310 as a shaft. For example, the roll motion may be a motion in which the surgical instrument rotates in the clockwise or counterclockwise direction around the extension direction of the connection part 310 (the X-axis direction of FIG. 11).

Meanwhile, the roll motion may mean a motion in which the end tool 100 rotates around the X-axis with respect to the connection part 310. For example, the roll motion may be a motion in which the end tool rotates in the clockwise or counterclockwise direction around the extension direction of the connection part 310 (the X-axis direction of FIG. 12).

The power transmission part 300 may connect the driving part 200 and the end tool 100, transmit the driving force from the driving part 200 to the end tool 100, and include a plurality of wires, pulleys, links, sections, gears, or the like.

Hereinafter, the end tool 100, the driving part 200, the power transmission part 300, and the like of the surgical instrument 30 of FIG. 11 will be described in more detail.

Hereinafter, the power transmission part 300 of the surgical instrument 30 of FIG. 11 will be described in more detail.

Referring to FIGS. 11 to 19, the power transmission part 300 of the surgical instrument 30 according to an embodiment of the present disclosure may include a plurality of wires 301, 302,303, 304, 305, and 306.

Here, the wires 301 and 305 may be paired to serve as first jaw wires. The wires 302 and 306 may be paired to serve as second jaw wires. Here, the components encompassing the wires 301 and 305, which are first jaw wires, and the wires 302 and 306, which are second jaw wires, may be referred to as jaw wires. In addition, the wires 303 and 304 may be paired to serve as pitch wires.

Here, in the drawings, a pair of wires are illustrated as being associated with a rotational motion of a first jaw 101, and a pair of wires are illustrated as being associated with a rotational motion of a second jaw 102, but an embodiment of the present disclosure is not limited thereto. For example, a pair of wires may be associated with a yaw motion, and a pair of wires may be associated with an actuation motion.

In addition, the power transmission part 300 of the surgical instrument 30 according to an embodiment of the present disclosure may include a coupling member 321, a coupling member 326, and the like, which are coupled to respective end portions of the wires in order to couple the wires and the pulleys. Here, each of the coupling members may have various shapes as necessary, such as a ball shape, a tube shape, and the like.

Here, the coupling member 321, which is a pitch wire coupling member, is coupled to the end portions of the wires 303 and 304, which are pitch wires, at the end tool 100 side to serve as a pitch wire-end tool coupling member. Meanwhile, although not illustrated in the drawings, a pitch wire-driving part coupling member (not shown) may be coupled to the end portions of the wires 303 and 304, which are pitch wires, at the driving part 200 side.

Meanwhile, the coupling member 326, which is a second jaw wire coupling member, is coupled to the end portions of the wires 302 and 306, which are second jaw wires, at the end tool 100 side to serve as a second jaw wire-end tool coupling member. Meanwhile, although not illustrated in the drawings, a second jaw wire-driving part coupling member (not shown) may be coupled to the end portions of the wires 302 and 306, which are second jaw wires, at the driving part 200 side.

Meanwhile, although not illustrated in the drawings, a coupling member (not shown) having the same shape as the second jaw wire coupling member 326 may be coupled to the end portions of the wires 301 and 305, which are first jaw wires, at the end tool 100 side to serve as a first jaw wire-end tool coupling member. Meanwhile, although not illustrated in the drawings, a first jaw wire-driving part coupling member (not shown) may be coupled to the end portions of the wires 301 and 305, which are first jaw wires, at the driving part 200 side.

Here, each of the coupling members is classified as being included in the power transmission part 300, but the coupling members may be classified such that the coupling member at the end tool 100 side may be included in the end tool 100, and the coupling member at the driving part 200 side may be included in the driving part 200.

The coupling relationship between the wires, the fastening members, and the respective pulley will be described in detail as follows.

First, the wires 302 and 306, which are second jaw wires, may be a single wire. The second jaw wire coupling member 326, which is a second jaw wire-end tool coupling member, is inserted at an intermediate point of the second jaw wire, which is a single wire, and the second jaw wire coupling member 326 is crimped and fixed, and then, both strands of the second jaw wire centered on the second jaw wire coupling member 326 may be referred to as the wire 302 and the wire 306, respectively.

Alternatively, the wires 302 and 306, which are second jaw wires, may also be formed as separate wires, and connected to each other by the second jaw wire coupling member 326.

In addition, by coupling the second jaw wire coupling member 326 to a pulley 121, the wires 302 and 306 may be fixedly coupled to the pulley 121. This allows the pulley 121 to rotate as the wires 302 and 306 are pulled and released.

Meanwhile, the second jaw wire-driving part coupling member (not shown) may be coupled to the end portions of the wires 302 and 306, which are opposite to the end portions to which the second jaw wire coupling member 326 is coupled. That is, the second jaw wire-driving part coupling member (not shown) may be fixed to each of the wires 302 and 306 by inserting the opposite end portions of the wires 302 and 306 into the second jaw wire-driving part coupling member (not shown) and crimping the coupling member (not shown).

In addition, by coupling the second jaw wire-driving part coupling member (not shown) coupled to the wires 302 and 306 to each of the pulley 221 and the pulley 222, the wire 302 and the wire 306 may be fixedly coupled to the pulley 221 and the pulley 222, respectively. As a result, when the pulley 221 and the pulley 222 are rotated by a motor or a human force, the pulley 121 of the end tool 100 may be rotated as the wire 302 and the wire 306 are pulled and released.

Here, a driving part second jaw pulley may include two pulleys of the pulley 221 and the pulley 222, and thus the second jaw wire-driving part coupling member may also include two coupling members. Alternatively, the driving part second jaw pulley includes one pulley, the second jaw wire-driving part coupling member also includes one coupling member, and the wires 302 and 306 may be coupled to one coupling member to be coupled to one driving part second jaw pulley.

In the same manner, the wire 301 and the wire 305, which are first jaw wires, are coupled to the first jaw wire-end tool coupling member (not shown) and the first jaw wire-driving part coupling member (not shown), respectively. In addition, the first jaw wire-end tool coupling member (not shown) is coupled to a pulley 111, and the first jaw wire-driving part coupling member (not shown) is coupled to a pulley 211 and a pulley 212. As a result, when the pulleys 211 and 212 are rotated by a motor or a human force, the pulley 111 of the end tool 100 may be rotated as the wire 301 and the wire 305 are pulled and released.

In the same manner, each of one end portions of the wires 303 and 304, which are pitch wires, is coupled to the pitch wire coupling member 321, which is a pitch wire-end tool coupling member, and each of the other end portions of the wires 303 and 304 are coupled to the pitch wire-driving part coupling member (not shown). In addition, the pitch wire coupling member 321 is coupled to a pulley 131, and the pitch wire-driving part coupling member (not shown) is coupled to a pulley 231. As a result, when the pulley 231 is rotated by a motor or a human force, the pulley 131 of the end tool 100 may be rotated as the wire 303 and the wire 304 are pulled and released.

As a result, the wire 301 and the wire 305, which are both strands of the first jaw wire, are coupled to a coupling member 323, which is a first jaw wire-end tool coupling member, and the first jaw wire-driving part coupling member (not shown) so as to form as a whole a closed loop. Similarly, the second jaw wire and the pitch wire may each be formed to form a closed loop.

Hereinafter, the end tool 100 of the surgical instrument 30 of FIG. 11 will be described in more detail.

FIGS. 12 and 13 are perspective views of the end tool of the surgical instrument of FIG. 11, and FIGS. 14A to 14B is a plan view of the end tool of the surgical instrument of FIG. 11. Here, FIG. 12 illustrates a state in which an end tool hub 106 and a pitch hub 107 are coupled, and FIG. 13 illustrates a state in which the end tool hub 106 and the pitch hub 107 are removed.

Referring to FIGS. 12 to 14, the end tool 100 according to an embodiment of the present disclosure includes a pair of jaws for performing a grip motion, that is, the first jaw 101 and the second jaw 102. Here, each of the first jaw 101 and the second jaw 102, or a component encompassing the first jaw 101 and the second jaw 102 may be referred to as a jaw 103.

Further, the end tool 100 may include the pulley 111, a pulley 112, a pulley 113, a pulley 114, a pulley 115, and a pulley 116 that are related to a rotational motion of the first jaw 101. In addition, the end tool 100 may include the pulley 121, a pulley 122, a pulley 123, a pulley 124, a pulley 125, and a pulley 126 that are related to a rotational motion of the second jaw 102.

Here, in the drawings, one group of pulleys are illustrated as being associated with a rotational motion of the first jaw 101, and one group of pulleys are illustrated as being associated with a rotational motion of the second jaw 102, but an embodiment of the present disclosure is not limited thereto. For example, one group of pulleys in the end tool may be associated with a yaw motion, and one group of pulleys in the end tool may be associated with an actuation motion. Here, the pulleys included in the end tool 100, including the pulleys described above, may be collectively referred to as end tool pulleys.

Meanwhile, the pulleys facing each other are illustrated in the drawings as being formed parallel to each other, but an embodiment of the present disclosure is not limited thereto, and each of the pulleys may be variously formed with a position and a size suitable for the configuration of the end tool.

Further, the end tool 100 according to an embodiment of the present disclosure may include the end tool hub 106 and the pitch hub 107.

The rotation shaft 141 and a rotation shaft 142, which will be described later, may be inserted through the end tool hub 106, and the end tool hub 106 may internally accommodate at least some of the first jaw 101 and the second jaw 102, which are axially coupled to the rotation shaft 141. In addition, the end tool hub 106 may internally accommodate at least some of the pulley 112 and the pulley 122 that are axially coupled to the rotation shaft 142.

In addition, the pulley 131 serving as an end tool pitch pulley may be formed at one end portion of the end tool hub 106. As shown in FIG. 12, the pulley 131 may be formed as a separate member from the end tool hub 106 and coupled to the end tool hub 106. Alternatively, although not illustrated in the drawings, the pulley 131 may be integrally formed with the end tool hub 106 as one body. That is, one end portion of the end tool hub 106 is formed in a disk shape or a semi-circular shape such as a pulley, and a groove around which a wire can be wound may be formed on an outer circumferential surface thereof. The wires 303 and 304 described above are coupled to the pulley 131 serving as an end tool pitch pulley, and a pitch motion may be performed as the pulley 131 is rotated around the rotation shaft 143.

The rotation shaft 143 and a rotation shaft 144, which will be described later, may be inserted through the pitch hub 107, and the pitch hub 107 may be axially coupled to the end tool hub 106 and the pulley 131 by the rotation shaft 143. Thus, the end tool hub 106 and the pulley 131 (coupled thereto) may be formed to be rotatable around the rotation shaft 143 with respect to the pitch hub 107.

Further, the pitch hub 107 may internally accommodate at least some of the pulley 113, the pulley 114, the pulley 123, and the pulley 124 that are axially coupled to the rotation shaft 143. In addition, the pitch hub 107 may internally accommodate at least some of the pulley 115, the pulley 116, the pulley 125, and the pulley 126 that are axially coupled to the rotation shaft 144.

Further, the end tool 100 according to an embodiment of the present disclosure may include the rotation shaft 141, the rotation shaft 142, the rotation shaft 143, and the rotation shaft 144. As described above, the rotation shaft 141 and the rotation shaft 142 may be inserted through the end tool hub 106, and the rotation shaft 143 and the rotation shaft 144 may be inserted through the pitch hub 107.

The rotation shaft 141, the rotation shaft 142, the rotation shaft 143, and the rotation shaft 144 may be arranged sequentially from a distal end 104 of the end tool 100 toward a proximal end 105 thereof. Accordingly, starting from the distal end 104, the rotation shaft 141 may be referred to as a first pin, the rotation shaft 142 may be referred to as a second pin, the rotation shaft 143 may be referred to as a third pin, and the rotation shaft 144 may be referred to as a fourth pin.

Here, the rotation shaft 141 may function as an end tool jaw pulley rotation shaft, the rotation shaft 142 may function as an end tool jaw auxiliary pulley rotation shaft, the rotation shaft 143 may function as an end tool pitch rotation shaft, and the rotation shaft 144 may function as an end tool pitch auxiliary rotation shaft of the end tool 100.

Each of the rotation shafts 141, 142, 143, and 144 may be fitted into one or more pulleys, which will be described in detail below.

The pulley 111 functions as an end tool first jaw pulley, and the pulley 121 functions as an end tool second jaw pulley, and these two components may be collectively referred to as end tool jaw pulleys.

The pulley 111 and the pulley 121, which are end tool jaw pulleys, are formed to face each other, and are formed to be rotatable independently of each other around the rotation shaft 141, which is an end tool jaw pulley rotation shaft. Here, in the drawings, it is illustrated that the pulley 111 and the pulley 121 are formed to rotate around one rotation shaft 141, but it is of course possible that each end tool jaw pulley may be formed to be rotatable around a separate shaft. Here, the first jaw 101 may be fixedly coupled to the pulley 111 and rotated together with the pulley 111, and the second jaw 102 may be fixedly coupled to the pulley 121 and rotated together with the pulley 121. Yaw and actuation motions of the end tool 100 are performed according to the rotation of the pulley 111 and the pulley 121. That is, when the pulley 111 and the pulley 121 are rotated in the same direction around the rotation shaft 141, the yaw motion is performed, and when the pulley 111 and the pulley 121 are rotated in opposite directions around the rotation shaft 141, the actuation motion is performed.

Here, the first jaw 101 and the pulley 111 may be formed as separate members and coupled to each other, or the first jaw 101 and the pulley 111 may be integrally formed as one body. Similarly, the second jaw 102 and the pulley 121 may be formed as separate members and coupled to each other, or the second jaw 102 and the pulley 121 may be integrally formed as one body.

The pulley 112 functions as an end tool first jaw auxiliary pulley, and the pulley 122 functions as an end tool second jaw auxiliary pulley, and these two components may be collectively referred to as end tool jaw auxiliary pulleys.

Specifically, the pulley 112 and the pulley 122, which are end tool jaw auxiliary pulleys, may be additionally provided on one side of the pulley 111 and one side of the pulley 121, respectively. In other words, the pulley 112, which is an auxiliary pulley, may be disposed between the pulley 111 and the pulley 113/pulley 114. In addition, the pulley 122, which is an auxiliary pulley, may be disposed between the pulley 121 and the pulley 123/pulley 124. The pulley 112 and the pulley 122 may be formed to be rotatable independently of each other around the rotation shaft 142. Here, in the drawings, it is illustrated that the pulley 112 and the pulley 122 are formed to rotate around one rotation shaft 142, but it is of course possible that each of the pulley 112 and the pulley 122 may be formed to be rotatable around a separate shaft. Such auxiliary pulleys will be described in more detail later.

The pulley 113 and the pulley 114 function as end tool first jaw pitch main pulleys, and the pulley 123 and the pulley 124 function as end tool second jaw pitch main pulleys, and these two components may be collectively referred to as end tool jaw pitch main pulleys.

The pulley 115 and the pulley 116 function as end tool first jaw pitch sub-pulleys, and the pulley 125 and the pulley 126 function as end tool second jaw pitch sub-pulleys, and these two components may be collectively referred to as end tool jaw pitch sub-pulleys.

Hereinafter, components related to the rotation of the pulley 111 will be described.

The pulley 113 and the pulley 114 function as end tool first jaw pitch main pulleys. That is, the pulley 113 and the pulley 114 function as main rotation pulleys for a pitch motion of the first jaw 101. Here, the wire 301, which is a first jaw wire, is wound around the pulley 113, and the wire 305, which is a first jaw wire, is wound around the pulley 114.

The pulley 115 and the pulley 116 function as end tool first jaw sub-pulleys. That is, the pulley 115 and the pulley 116 function as sub rotation pulleys for a pitch motion of the first jaw 101. Here, the wire 301, which is a first jaw wire, is wound around the pulley 115, and the wire 305, which is a first jaw wire, is wound around the pulley 116.

Here, the pulley 113 and the pulley 114 are disposed on one side of the pulley 111 and the pulley 112 to face each other. Here, the pulley 113 and the pulley 114 are formed to be rotatable independently of each other around the rotation shaft 143 that is an end tool pitch rotation shaft. In addition, the pulley 115 and the pulley 116 are disposed on one side of the pulley 113 and on one side of the pulley 114, respectively, to face each other. Here, the pulley 115 and the pulley 116 are formed to be rotatable independently of each other around the rotation shaft 144 that is an end tool pitch auxiliary rotation shaft. Here, in the drawings, it is illustrated that the pulley 113, the pulley 115, the pulley 114, and the pulley 116 are all formed to be rotatable around a Y-axis direction, but an embodiment of the present disclosure is not limited thereto, and the rotation axes of the respective pulleys may be formed in various directions according to configurations thereof.

The wire 301, which is a first jaw wire, is sequentially wound to make contact with at least portions of the pulley 115, the pulley 113, and the pulley 111. In addition, the wire 305 connected to the wire 301 by the first jaw wire-end tool coupling member 323 is sequentially wound to make contact with at least portions of the pulley 111, the pulley 112, the pulley 114, and the pulley 116 in turn.

Viewed from another perspective, the wires 301 and 305, which are first jaw wires, are sequentially wound to make contact with at least portions of the pulley 115, the pulley 113, the pulley 111, the pulley 112, the pulley 114, and the pulley 116 and are formed to move along the above pulleys while rotating the above pulleys.

Accordingly, when the wire 301 is pulled in the direction of an arrow of the wire 301 of FIGS. 14A to 14B, a coupling member (not shown) to which the wire 301 is coupled and the pulley 111 coupled to the coupling member (not shown) are rotated in an arrow L direction of FIGS. 14A to 14B. In contrast, when the wire 305 is pulled in the direction of an arrow of the wire 305 of FIGS. 14A to 14B, a coupling member (not shown) to which the wire 305 is coupled and the pulley 111 coupled to the coupling member (not shown) are rotated in an arrow R direction of FIGS. 14A to 14B.

Hereinafter, the pulley 112 and the pulley 122 serving as auxiliary pulleys will be described in more detail.

The pulley 112 and the pulley 122 may serve to increase rotation angles of the first jaw 101 and the second jaw 102, respectively, by coming into contact with the wire 305, which is a first jaw wire, and the wire 302, which is a second jaw wire, and changing the arrangement paths of the wires 305 and 302 to a certain extent.

That is, when the auxiliary pulleys are not disposed, each of the first jaw and the second jaw may be rotated up to a right angle, but in an embodiment of the present disclosure, the pulley 112 and the pulley 122, which are auxiliary pulleys, are additionally provided, so that the maximum rotation angle may be increased by θ as shown in FIGS. 14A to 14B. This enables a motion of the two jaws of the end tool 100 being opened for an actuation motion while the two jaws are yaw-rotated by 90° in the L direction. This is because the second jaw 102 is rotated by the additional angle θ as shown in FIG. 12. Similarly, an actuation motion is possible even when the two jaws are yaw-rotated in the R direction. In other words, a feature of increasing the range of yaw rotation in which an actuation motion is possible may be obtained through the pulley 112 and the pulley 122.

This will be described in more detail as follows.

When the auxiliary pulleys are not disposed, since the first jaw wire is fixedly coupled to the end tool first jaw pulley, and the second jaw wire is fixedly coupled to the end tool second jaw pulley, each of the end tool first jaw pulley and the end tool second jaw pulley may be rotated up to 90°. In this case, when the actuation motion is performed while the first jaw and the second jaw are located at a 90° line, the first jaw may be opened, but the second jaw may not be rotated beyond 90°. Accordingly, when the first jaw and the second jaw perform a yaw motion over a certain angle, there was a problem that the actuation motion is not smoothly performed.

In order to address such a problem, in the surgical instrument 30 according to an embodiment of the present disclosure, the pulley 112 and the pulley 122, which are auxiliary pulleys, are additionally disposed at one side of the pulley 111 and one side of the pulley 121, respectively. As described above, as the arrangement paths of the wire 305, which is a first jaw wire, and the wire 302, which is a second jaw wire, are changed to a certain extent by disposing the pulley 112 and the pulley 122, a tangential direction of the wires 305 and 302 is changed, and accordingly, the second jaw wire coupling member 326 for coupling the wire 302 and the pulley 121 may be rotated up to a line N of FIGS. 14A to 14B. That is, the second jaw wire coupling member 326, which is a coupling part of the wire 302 and the pulley 121, is rotatable until the second jaw wire coupling member 326 is located on a common internal tangent of the pulley 121 and the pulley 122. Similarly, the first jaw wire-end tool coupling member 323, which is a coupling part of the wire 305 and the pulley 111, is rotatable until the first jaw wire-end tool coupling member 323 is located on a common internal tangent of the pulley 111 and the pulley 112, so that the range of rotation in the L direction may be increased.

In other words, by the pulley 112, the wires 301 and 305, which are two strands of the first jaw wire wound around the pulley 111, are disposed at one side with respect to a plane perpendicular to the Y-axis and passing through the X-axis. Simultaneously, by the pulley 122, the wires 302 and 306, which are two strands of the second jaw wire wound around the pulley 121, are disposed at the other side with respect to the plane perpendicular to the Y-axis and passing through the X-axis.

In other words, the pulley 113 and the pulley 114 are disposed at one side with respect to the plane perpendicular to the Y-axis and passing through the X-axis, and the pulley 123 and the pulley 124 are disposed at the other side with respect to the plane perpendicular to the Y-axis and passing through the X-axis.

In other words, the wire 305 is located on the internal tangent of the pulley 111 and the pulley 112, and the rotation angle of the pulley 111 is increased by the pulley 112. In addition, the wire 302 is located on the internal tangent of the pulley 121 and the pulley 122, and the rotation angle of the pulley 121 is increased by the pulley 122.

According the above-described embodiment of the present disclosure, as the rotation radii of the jaw 101 and the jaw 102 increase, an effect of increasing a yaw motion range in which a normal opening/closing actuation motion is performed may be obtained.

Next, components related to the rotation of the pulley 121 will be described.

The pulley 123 and the pulley 124 function as end tool second jaw pitch main pulleys. That is, the pulley 123 and the pulley 124 function as main rotation pulleys for a pitch motion of the second jaw 102. Here, the wire 306, which is a second jaw wire, is wound around the pulley 123, and the wire 302, which is a second jaw wire, is wound around the pulley 124.

The pulley 125 and the pulley 126 function as end tool second jaw sub-pulleys. That is, the pulley 125 and the pulley 126 function as sub rotation pulleys for a pitch motion of the second jaw 102. Here, the wire 306, which is a second jaw wire, is wound around the pulley 125, and the wire 302, which is a second jaw wire, is wound around the pulley 126.

On one side of the pulley 121, the pulley 123 and the pulley 124 are disposed to face each other. Here, the pulley 123 and the pulley 124 are formed to be rotatable independently of each other around the rotation shaft 143 that is an end tool pitch rotation shaft. In addition, the pulley 125 and the pulley 126 are disposed on one side of the pulley 123 and one side of the pulley 124, respectively, to face each other. Here, the pulley 125 and the pulley 126 are formed to be rotatable independently of each other around the rotation shaft 144, which is an end tool pitch auxiliary rotation shaft. Here, in the drawings, it is illustrated that all of the pulley 123, the pulley 125, the pulley 124, and the pulley 126 are formed to be rotatable around the Y-axis direction, but an embodiment of the present disclosure is not limited thereto, and the rotation axes of the respective pulleys may be formed in various directions according to configurations thereof.

The wire 306, which is a second jaw wire, is sequentially wound to make contact with at least portions of the pulley 125, the pulley 123, and the pulley 121. In addition, the wire 302 connected to the wire 306 by the second jaw wire coupling member 326 is sequentially wound to make contact with at least portions of the pulley 121, the pulley 122, the pulley 124, and the pulley 126.

Viewed from another perspective, the wires 306 and 302, which are second jaw wires, are sequentially wound to make contact with at least portions of the pulley 125, the pulley 123, the pulley 121, the pulley 122, the pulley 124, and the pulley 126, and are formed to move along the above pulleys while rotating the above pulleys.

Accordingly, when the wire 306 is pulled in the direction of an arrow of the wire 306 of FIGS. 14A to 14B, the second jaw wire coupling member 326 to which the wire 306 is coupled and the pulley 121 coupled to the second jaw wire coupling member 326 are rotated in the arrow R direction of FIGS. 14A to 14B. In contrast, when the wire 302 is pulled in the direction of an arrow of the wire 302 of FIG. 14A, the second jaw wire coupling member 326 to which the wire 302 is coupled and the pulley 121 coupled to the second jaw wire coupling member 326 are rotated in the arrow L direction of FIGS. 14A to 14B.

Hereinafter, a pitch motion of the present disclosure will be described in more detail.

First, for the pitch motion, at the end tool 100 side, the pulley 113, the pulley 114, the pulley 123, and the pulley 124, which are end tool jaw pitch main pulleys, are formed to be rotatable around the rotation shaft 143. Meanwhile, in a direction of the proximal end 105 of the end tool jaw pitch main pulley, the pulley 115, the pulley 116, the pulley 125, and the pulley 126, which are end tool jaw pitch sub-pulleys, are formed to be rotatable around the rotation shaft 144.

In addition, based on a plane perpendicular to the rotation shaft 141 and including the rotation shaft 143 (i.e., an XY plane), the wires 301 and 305, which are two strands of the first jaw wire, are located on the same side with respect to the XY plane. That is, the wire 301 and the wire 305 are formed to pass through lower sides of the pulley 113 and the pulley 114, which are end tool jaw pitch main pulleys, and upper sides of the pulley 115 and the pulley 116, which are end tool jaw pitch sub-pulleys.

Similarly, the wires 302 and 306, which are two strands of the second jaw wire, are located on the same side with respect to the XY plane. That is, the wires 302 and 306 are formed to pass through upper sides of the pulley 123 and the pulley 124, which are end tool jaw pitch main pulleys, and lower sides of the pulley 125 and the pulley 126, which are end tool jaw pitch sub-pulleys.

In addition, in the wires 301 and 305 that are two strands of the first jaw wire, when the wire 301 is pulled toward the arrow of the wire 301 of FIGS. 14A to 14B and simultaneously the wire 305 is pulled toward the arrow of the wire 305 of FIGS. 14A to 14B (i.e., when both strands of the first jaw wire are pulled in the same direction), as shown in FIG. 12, since the wires 301 and 305 are wound around lower portions of the pulleys 113 and 114, which are rotatable around the rotation shaft 143 that is an end tool pitch rotation shaft, the pulley 111 to which the wire 301 and the wire 305 are fixedly coupled, and the end tool hub 106 to which the pulley 111 is coupled are rotated together as a whole in a counterclockwise direction around the rotation shaft 143, as a result, the end tool 100 performs the pitch motion while rotating downward. At this time, since the second jaw 102 and the wires 302 and 306 fixedly coupled thereto are wound around the upper portions of the pulleys 123 and 124 rotatable around the rotation shaft 143, the wires 302 and 306 are unwound in opposite directions of the arrows of the wires 302 and 306, respectively.

In contrast, in the wires 302 and 306 that are two strands of the second jaw wire, when the wire 302 is pulled toward the arrow of the wire 302 of FIGS. 14A to 14B and simultaneously the wire 306 is pulled toward the arrow of the wire 306 of FIGS. 14A to 14B (i.e., when both strands of the second jaw wire are pulled in the same direction), as shown in FIG. 12, since the wires 302 and 306 are wound upward lower portions of the pulleys 123 and 124, which are rotatable around the rotation shaft 143 that is an end tool pitch rotation shaft, the pulley 121 to which the wire 302 and the wire 306 are fixedly coupled, and the end tool hub 106 to which the pulley 121 is coupled are rotated together as a whole in a clockwise direction around the rotation shaft 143. As a result, the end tool 100 performs the pitch motion while rotating upward. At this time, since the first jaw 101 and the wires 301 and 305 fixedly coupled thereto are wound downward the lower portions of the pulleys 113 and 114 rotatable around the rotation shaft 143, the wires 302 and 306 are moved in opposite directions of the arrows of the wires 301 and 305, respectively.

Viewed from another perspective, it may be also described that both strands of each jaw wire are moved simultaneously in the same direction when the end tool 100 is pitch-rotated.

Meanwhile, the end tool 100 of the surgical instrument 30 of the present disclosure may further include the pulley 131, which is an end tool pitch pulley, the driving part 200 may further include the pulley 231, which is a driving part pitch pulley, and the power transmission part 300 may further include the wire 303 and the wire 304 that are pitch wires. Specifically, the pulley 131 of the end tool 100 is rotatable around the rotation shaft 143, which is an end tool pitch rotation shaft, and may be integrally formed with the end tool hub 106 (or fixedly coupled to the end tool hub 106) as one body. In addition, the wires 303 and 304 may serve to connect the pulley 131 of the end tool 100 to the pulley 231 of the driving part 200.

Thus, when the pulley 231 of the driving part 200 is rotated, the rotation of the pulley 231 is transmitted to the pulley 131 of the end tool 100 via the wires 303 and 304, which causes the pulley 131 to also be rotated, and as a result, the end tool 100 performs a pitch motion while rotating.

That is, in the surgical instrument 30 according to an embodiment of the present disclosure, by providing the pulley 131 of the end tool 100, the pulley 231 of the driving part 200, and the wires 303 and 304 of the power transmission part 300 to transmit power for a pitch motion, the driving force for a pitch motion from the driving part 200 may be more completely transmitted to the end tool 100, thereby improving operation reliability.

Here, a diameter of each of the pulley 113, the pulley 114, the pulley 123, and the pulley 124, which are end tool jaw pitch main pulleys, and a diameter of the pulley 131, which is an end tool pitch pulley, may be the same as each other or different from each other. At this time, a ratio of the diameter of the end tool jaw pitch main pulley to the diameter of the end tool pitch pulley may be the same as a ratio of a diameter of a driving part relay pulley of the driving part 200, which will be described later, to a diameter of a driving part pitch pulley 231. This will be described in detail later.

Hereinafter, the driving part 200 of the surgical instrument 30 of FIG. 11 will be described in more detail.

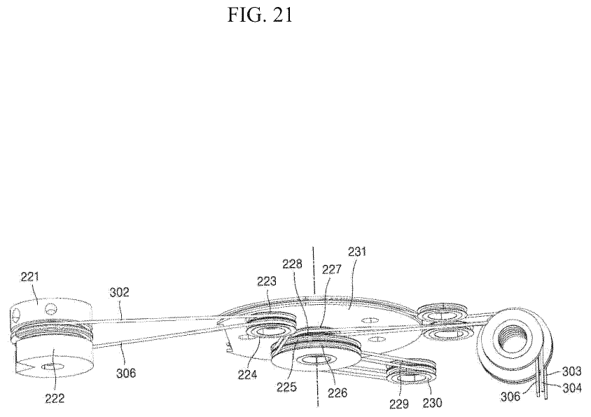

Referring to FIGS. 15 to 21, the driving part 200 of the surgical instrument 30 according to an embodiment of the present disclosure may include the pulley 211, the pulley 212, a pulley 213, a pulley 214, a pulley 215, a pulley 216, a pulley 217, a pulley 218, a pulley 219, and a pulley 220, which are related to a rotational motion of the first jaw 101. In addition, the driving part 200 may include the pulley 221, the pulley 222, a pulley 223, a pulley 224, a pulley 225, a pulley 226, a pulley 227, a pulley 228, a pulley 229, and a pulley 230, which are related to a rotational motion of the second jaw 102.

Here, the pulleys facing each other are illustrated in the drawings as being formed parallel to each other, but an embodiment of the present disclosure is not limited thereto, and each of the pulleys may be variously formed with a position and a size suitable for the configuration of the driving part.

In addition, the driving part 200 of the surgical instrument 30 according to an embodiment of the present disclosure may further include the pulley 231 serving as a driving part pitch pulley, and a pitch-yaw connector 232 configured to connect the pulley 231 to the above-described jaw pulleys of the driving part.

Further, the driving part 200 according to an embodiment of the present disclosure may include a rotation shaft 241, a rotation shaft 242, a rotation shaft 243, a rotation shaft 244, a rotation shaft 245, and a rotation shaft 246. Here, the rotation shaft 241 may function as a first jaw rotation shaft of the driving part, and the rotation shaft 242 may function as a second jaw rotation shaft of the driving part. In addition, the rotation shaft 243 may function as a driving part pitch rotation shaft, and the rotation shaft 244 may function as a driving part roll rotation shaft. In addition, the rotation shaft 245 may function as a driving part first jaw auxiliary rotation shaft of the driving part, and the rotation shaft 246 may function as a driving part second jaw auxiliary rotation shaft. Each of the rotation shafts 241, 242, 243, 244, 245, and 246 may be fitted into one or more pulleys, which will be described in detail later.

In addition, the driving part 200 according to an embodiment of the present disclosure may include a motor coupling part 251, a motor coupling part 252, a motor coupling part 253, and a motor coupling part 254. Here, the motor coupling part 251 may function as a first jaw driving motor coupling part, the motor coupling part 252 may function as a second jaw driving motor coupling part, the motor coupling part 253 may function as a pitch driving motor coupling part, and the motor coupling part 254 may function as a roll driving motor coupling part. Here, each of the motor coupling parts 251, 252, 253, and 254 may be provided in the form of a rotatable flat plate, in which one or more coupling holes, to which a motor (not shown) may be coupled, may be formed.

The motor coupling parts 251, 252, 253, and 254 of the driving part 200 described above are coupled to motors (not shown) formed in the robot arm units 21, 22, and 23, respectively, so that the driving part 200 is operated by driving the motors (not shown).

In addition, the driving part 200 according to an embodiment of the present disclosure may include a gear 261, a gear 262, a gear 263, and a gear 264. Here, the gear 261 and the gear 262 may function as pitch driving gears, and the gear 263 and the gear 264 may function as roll driving gears.

Hereinafter, each component will be described in more detail.

The pulley 211 and the pulley 212 may function as driving part first jaw pulleys, and the pulley 221 and the pulley 222 may function as driving part second jaw pulleys, and these components may be collectively referred to as driving part jaw pulleys.

Here, it is illustrated in the drawings that the pulley 211 is associated with a rotational motion of the first jaw 101 of the end tool 100, and the pulley 221 is associated with a rotational motion of the second jaw 102 of the end tool 100, but an embodiment of the present disclosure is not limited thereto. For example, one group of pulleys in the driving part may be associated with a yaw motion, and one group of pulleys in the driving part may be associated with an actuation motion. Thus, the pulley 211 and the pulley 212 may be collectively referred to as driving part driving pulleys. In addition, in the other pulleys, one group of pulleys may also be associated with a yaw motion, and one group of pulleys may also be associated with an actuation motion.

The pulley 213 and the pulley 214 may function as driving part first jaw auxiliary pulleys, and the pulley 223 and the pulley 224 may function as driving part second jaw auxiliary pulleys, and these components may be collectively referred to as driving part auxiliary pulleys.

The pulley 215 and the pulley 216 may function as driving part first jaw first relay pulleys, and the pulley 217 and the pulley 218 may function as driving part first jaw second relay pulleys, and these components may be collectively referred to as driving part first jaw relay pulleys. Meanwhile, the pulley 225 and the pulley 226 may function as driving part second jaw first relay pulleys, and the pulley 227 and the pulley 228 may function as driving part second jaw second relay pulleys, and these components may be collectively referred to as driving part second jaw relay pulleys. Meanwhile, the pulley 215, the pulley 216, the pulley 225, and the pulley 226 may be collectively referred to as driving part first relay pulleys, and the pulley 217, the pulley 218, the pulley 227, and the pulley 228 may be collectively referred to as driving part second relay pulleys. Furthermore, the pulley 215, the pulley 216, the pulley 217, the pulley 218, the pulley 225, the pulley 226, the pulley 227, and the pulley 228 may be collectively referred to as driving part relay pulleys.

Here, it is illustrated in the drawings that two pulleys are paired to form the driving part relay pulleys for each jaw, but an embodiment of the present disclosure is not limited thereto. For example, it is illustrated that the pulley 215, which is a driving part first jaw first relay pulley, and the pulley 217, which is a driving part first jaw second relay pulley, are formed as a pair, and the wire 301 sequentially passes through the pulley 215 and the pulley 217. However, the driving part first jaw relay pulley may be configured with not just two pulleys but also with three or more pulleys.

Meanwhile, the pulley 219 and the pulley 220 may function as driving part first jaw satellite pulleys, and the pulley 229 and the pulley 230 may function as driving part second jaw satellite pulleys, and these two components may be collectively referred to as driving part satellite pulleys.

A plurality of rotation shafts including the driving part first jaw rotation shaft 241, the driving part second jaw rotation shaft 242, the driving part pitch rotation shaft 243, the driving part roll rotation shaft 244, the driving part first jaw auxiliary rotation shaft 245, and the driving part second jaw auxiliary rotation shaft 246 may be formed on a first surface of a base plate 201. In addition, a plurality of relay pulleys 202 are formed on the first surface of the base plate 201, and may serve to redirect the wires 301, 302, 303, 304, 305, and 306 entering the driving part 200 through the connection part 310 toward the pulley 231.

Further, the connection part 310 in the form of a shaft is coupled to a second surface of the base plate 201 opposite to the first surface, and the first jaw motor coupling part 251, the second jaw driving motor coupling part 252, the pitch driving motor coupling part 253, and the roll driving motor coupling part 254, to which the motors (not shown) for driving the pulleys are coupled, may be formed on the second surface.

Here, each rotation shaft and each motor coupling part may be directly connected or indirectly connected to each other via a gear.

In an example, by directly coupling the first jaw motor coupling part 251 to the driving part first jaw rotation shaft 241, when the first jaw motor coupling part 251 coupled to a first jaw driving motor (not shown) is rotated, the driving part first jaw rotation shaft 241 directly coupled to the first jaw motor coupling part 251 may be rotated together. Similarly, by directly coupling the second jaw driving motor coupling part 252 to the driving part second jaw rotation shaft 242, when the second jaw driving motor coupling part 252 coupled to a second jaw driving motor (not shown) is rotated, the driving part second jaw rotation shaft 242 directly coupled to the second jaw driving motor coupling part 252 may be rotated together.

In another example, when viewed from a plane perpendicular to the driving part pitch rotation shaft 243, the pitch driving motor coupling part 253 and the driving part pitch rotation shaft 243 may be disposed to be spaced apart from each other by a certain extent. In addition, the pitch driving motor coupling part 253 and the driving part pitch rotation shaft 243 may be connected to each other by the gears 261 and 263, which are pitch driving gears.

Similarly, when viewed from a plane perpendicular to the driving part roll rotation shaft 244, the roll driving motor coupling part 254 and the driving part roll rotation shaft 244 may be disposed to be spaced apart from each other by a certain extent. In addition, the roll driving motor coupling part 254 and the driving part roll rotation shaft 244 may be connected to each other by the gears 263 and 264, which are roll driving gears.

As such, some motor coupling parts are configured to be directly connected to the rotation shafts, respectively, and the remaining motor coupling parts are configured to be indirectly connected to the rotation shafts, respectively, because the coupling position and direction between the surgical instrument 30 and the slave robot 20 should be considered. That is, the rotation shaft that is not affected by the coupling position with the slave robot 20 is directly connected to the motor coupling part, whereas the rotation shaft that may cause interference with the coupling position with the slave robot 20 may be indirectly connected to the motor coupling part.

It is illustrated in the drawings that the first jaw motor coupling part 251 and the second jaw driving motor coupling part 252 are directly connected to the rotation shafts, respectively, and the pitch driving motor coupling part 253 and the roll driving motor coupling part 254 are indirectly connected, respectively, through the gears, but an embodiment of the present disclosure is not limited thereto, and various configurations are possible according to the coupling position and direction with the slave robot 20.

The pulleys 211 and 212, which are driving part first jaw pulleys, may be coupled to the driving part first jaw rotation shaft 241. Here, the pulleys 211 and 212 may be formed to rotate together with the driving part first jaw rotation shaft 241.

In addition, the driving part first jaw auxiliary rotation shaft 245 may be disposed in a region adjacent to the driving part first jaw rotation shaft 241. The pulleys 213 and 214, which are driving part first jaw auxiliary pulleys, may be coupled to the driving part first jaw auxiliary rotation shaft 245. Here, the pulleys 213 and 214 may be formed to be rotatable around the driving part first jaw auxiliary rotation shaft 245.

Here, it is illustrated in the drawings that the driving part first jaw pulley is formed of two pulleys 211 and 212, the wire 301 is coupled to one pulley 211, and the wire 305 is coupled to the other pulley 212. However, an embodiment of the present disclosure is not limited thereto, and the driving part first jaw pulley may be formed of one pulley, and both the wires 301 and 305 may be coupled to the one pulley.

As described above, the driving part first jaw rotation shaft 241 is coupled to the first jaw driving motor (not shown) by the first jaw motor coupling part 251, and thus, when the first jaw driving motor (not shown) rotates for driving the first jaw 101, the pulleys 211 and 212, which are driving part first jaw pulleys, are rotated together with the driving part first jaw rotation shaft 241, so that the wires 301 and 305, which are first jaw wires, are pulled or released.

The pulleys 221 and 222, which are driving part second jaw rotation shafts, may be coupled to the driving part second jaw rotation shaft 242. Here, the pulley 221 and the pulley 222 may be formed to rotate together with the driving part second jaw rotation shaft 242.

In addition, the driving part second jaw auxiliary rotation shaft 246 may be disposed in a region adjacent to the driving part second jaw rotation shaft 242. The pulleys 223 and 224, which are driving part second jaw auxiliary pulleys, may be coupled to the driving part first jaw auxiliary rotation shaft 245. Here, the pulleys 223 and 224 may be formed to be rotatable around the driving part second jaw auxiliary rotation shaft 246.

Here, it is illustrated in the drawings that the driving part second jaw pulley is formed of two pulleys 221 and 222, the wire 302 is coupled to one pulley 221, and the wire 306 is coupled to the other pulley 222. However, an embodiment of the present disclosure is not limited thereto, and the driving part second jaw pulley may be formed of one pulley, and both the wires 302 and 306 may be coupled to the one pulley.

As described above, the driving part second jaw rotation shaft 242 is coupled to the second jaw driving motor (not shown) by the second jaw driving motor coupling part 252, and thus, when the second jaw driving motor (not shown) rotates for driving the second jaw 102, the pulley 221 and the pulley 222, which are driving part second jaw pulleys, are rotated together with the driving part second jaw rotation shaft 242, so that the wires 302 and 306, which are second jaw wires, are pulled or released.

The pulley 231, which is a driving part pitch pulley, may be coupled to the driving part pitch rotation shaft 243. Here, the pulley 231 may be formed to rotate together with the driving part pitch rotation shaft 243.

As described above, the driving part pitch rotation shaft 243 is coupled to a pitch driving motor (not shown) by the pitch driving motor coupling part 253, and thus, when the pitch driving motor (not shown) rotates for a pitch motion, the wires 303 and 304, which are pitch wires, are pulled or released as the pulley 231, which is a driving part pitch pulley, is rotated together with the driving part pitch rotation shaft 243.

Meanwhile, the pulley 215, the pulley 216, the pulley 217, the pulley 218, the pulley 225, the pulley 226, the pulley 227, and the pulley 228, which are driving part relay pulleys, may be formed to be rotatable around the driving part pitch rotation shaft 243 by inserting the driving part pitch rotation shaft 243 therethrough. Here, the pulley 215, the pulley 216, the pulley 217, and the pulley 218, which are driving part first jaw relay pulleys, may be disposed on one surface side of the pulley 231 that is a pitch pulley, and the pulley 225, the pulley 226, the pulley 227, and the pulley 228, which are driving part second jaw relay pulleys, may be disposed on the other surface side of the pulley 231.

Viewed from another perspective, along the driving part pitch rotation shaft 243, the pulleys 225 and 226, which are driving part second jaw first relay pulleys, the pulleys 227 and 228, which are driving part second jaw second relay pulleys, the pulley 231, which is a driving part pitch pulley, and the pulleys 217 and 218, which are driving part first jaw second relay pulleys, and the pulleys 215 and 216, which are driving part first jaw first relay pulleys, are sequentially stacked and formed.

In addition, the pitch-yaw connector 232 may be coupled to the driving part pitch rotation shaft 243. The pitch-yaw connector 232 may be formed to rigidly connect the pulley 231, which is a driving part pitch pulley, to the pulley 219, the pulley 220, the pulley 229, and the pulley 230, which are driving part satellite pulleys to allow the driving part satellite pulleys to be revolved around the driving part pitch rotation shaft 243 when the pulley 231 is rotated. This will be described in detail later.

Here, the pitch-yaw connector 232 may be formed to rotate together with the driving part pitch rotation shaft 243. That is, the pulley 231 and the pitch-yaw connector 232 may be coupled to the driving part pitch rotation shaft 243, and may be rotated together with the driving part pitch rotation shaft 243.

Here, the pitch-yaw connector 232 may be described as being formed in an approximately Y-shape as shown in FIG. 17, or the pitch-yaw connector 232 may be described as being formed in a shape in which at least two extension portions 232a and 232b are formed to extend from the center thereof. In addition, a driving part first jaw satellite pulley central shaft 233 and a driving part second jaw satellite pulley central shaft 234 may be formed at end portions of the extension portions 232a and 232b, respectively.

In addition, the pulleys 219 and 220, which are driving part first jaw satellite pulleys, may be coupled to the driving part first jaw satellite pulley central shaft 233, and the pulleys 229 and 230, which are driving part second jaw satellite pulleys, may be coupled to the driving part second jaw satellite pulley central shaft 234.

As a result, when the pulley 231, which is a driving part pitch pulley, is rotated together with the driving part pitch rotation shaft 243, the pulley 219, the pulley 220, the pulley 229, and the pulley 230, which are driving part satellite pulleys, are revolved around the driving part pitch rotation shaft 243. In other words, it may be said that the driving part first jaw satellite pulley central shaft 233 and the driving part second jaw satellite pulley central shaft 234 are rotated around the driving part pitch rotation shaft 243 while maintaining a constant distance from the driving part pitch rotation shaft 243 in a state in which the driving part first jaw satellite pulley central shaft 233 and the driving part second jaw satellite pulley central shaft 234 are spaced apart from the driving part pitch rotation shaft 243 by a certain extent.

That is, the driving part satellite pulley is formed to be movable relative to the driving part relay pulley and the driving part pitch rotation shaft 243 so that a relative position of the driving part satellite pulley with respect to the driving part relay pulley and the driving part pitch rotation shaft 243 may be changed. On the other hand, the relative positions of the driving part pitch pulley 231 and the driving part relay pulley remain constant.

In addition, when the pulley 231, which is a driving part pitch pulley, is rotated around the driving part pitch rotation shaft 243, the pulley 219, the pulley 220, the pulley 229, and the pulley 230, which are driving part satellite pulleys, are moved relative to the pulley 231, which is a driving part pitch pulley, so that the overall lengths of the wire 301, the wire 302, the wire 305, and the wire 306, which are jaw wires, in the driving part 200 are changed.

The wire 301, which is a first jaw wire, is connected to the end tool 100 through the connection part 310 after being sequentially wound to make contact with at least portions of the pulley 211, the pulley 213, the pulley 215, the pulley 219, and the pulley 217 in a state in which one end portion of the wire 301 is coupled to the pulley 211 by the first jaw wire-driving part coupling member (not shown).

Viewed from another perspective, the wire 301, which is a first jaw wire, is connected to the end tool 100 through the connection part 310 after being sequentially passing through the driving part first jaw pulley 211, the driving part first jaw auxiliary pulley 213, the driving part first jaw first relay pulley 215, the driving part first jaw satellite pulley 219, and the driving part first jaw second relay pulley 217.

Viewed from another perspective, the wire 301, which is a first jaw wire, enters the driving part 200 after passing through the end tool 100 and the connection part 310, and then is fixedly coupled to the pulley 211, which is a driving part first jaw pulley after being sequentially wound around the pulley 217, the pulley 219, the pulley 215, and the pulley 213.

Meanwhile, the wire 305, which is a first jaw wire, is connected to the end tool 100 through the connection part 310 after being sequentially wound to make contact with at least portions of the pulley 212, the pulley 214, the pulley 216, the pulley 220, and the pulley 218 in a state in which one end portion of the wire 305 is coupled to the pulley 212 by the first jaw wire-driving part coupling member (not shown).

The wire 302, which is a second jaw wire, is connected to the end tool 100 through the connection part 310 after being sequentially wound to make contact with at least portions of the pulley 221, the pulley 223, the pulley 225, the pulley 229, and the pulley 227 in a state in which one end portion thereof is coupled to the pulley 221 by the second jaw wire-driving part coupling member (not shown).

Meanwhile, the wire 306, which is a second jaw wire, is connected to the end tool 100 through the connection part 310 after being sequentially wound to make contact with at least portions of the pulley 222, the pulley 224, the pulley 226, the pulley 230, and the pulley 228 in a state in which one end portion thereof is coupled to the pulley 222 by the second jaw wire-driving part coupling member (not shown).

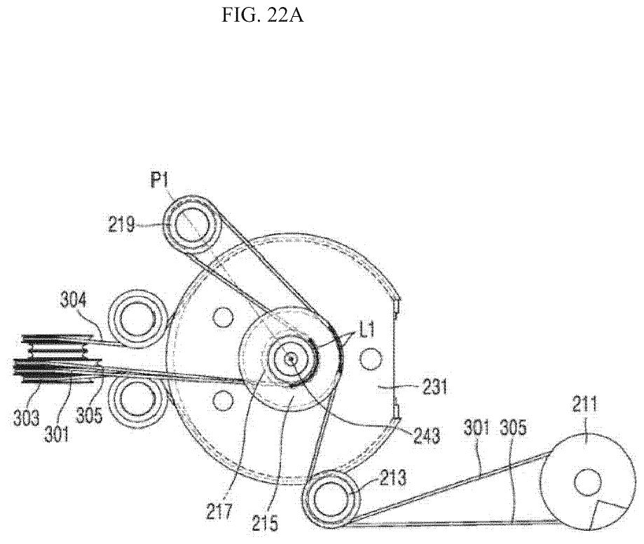

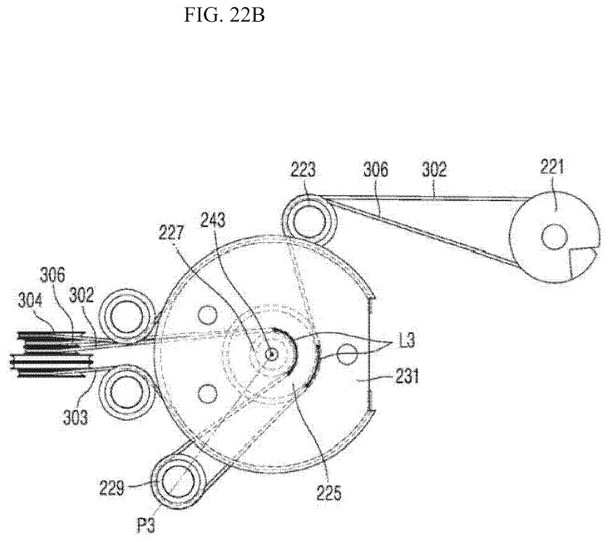

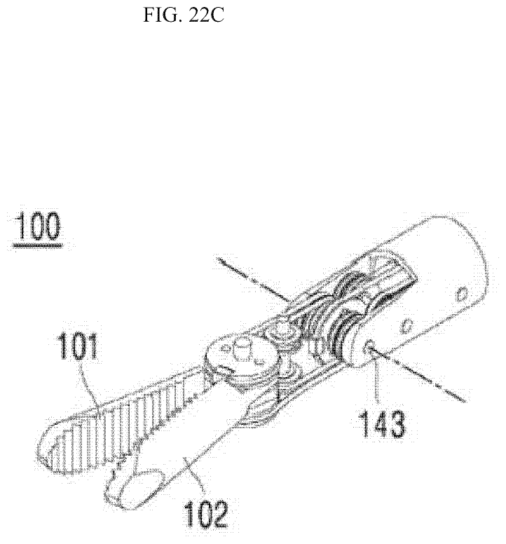

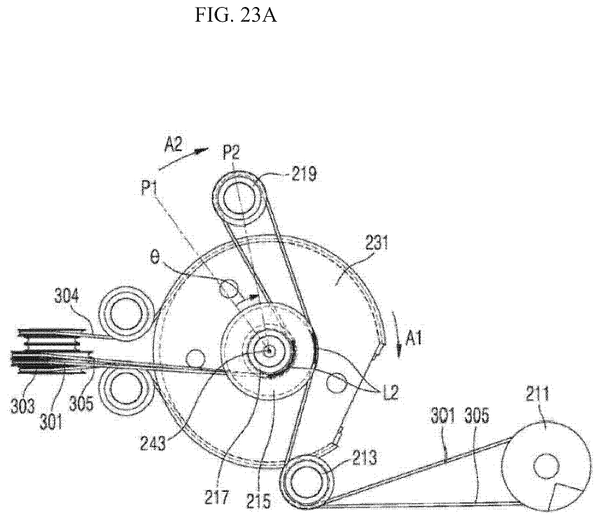

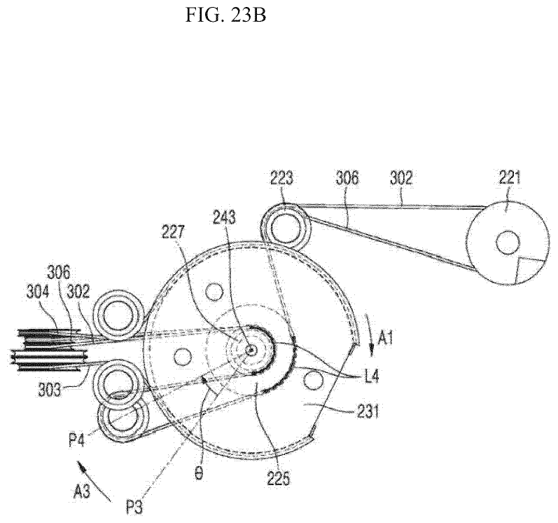

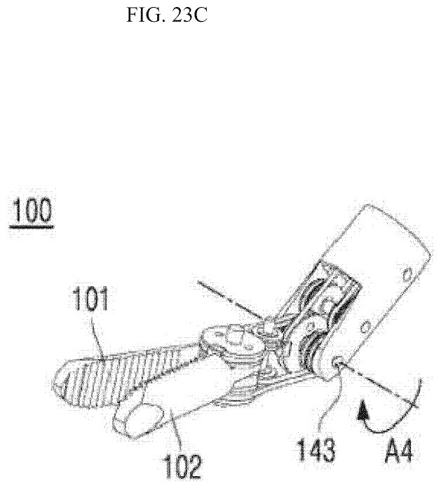

FIGS. 22A to 23C are diagrams illustrating a pitch motion of the surgical instrument illustrated in FIG. 11. Here, for convenience of description, only the pulleys and wires related to the rotation of the first jaw are illustrated in FIG. 22A and FIG. 23A, and only the pulleys and wires related to the rotation of the second jaw are illustrated in FIG. 22B and FIG. 23B. In addition, FIG. 22C and FIG. 23C illustrate a pitch motion of the end tool according to a pitch motion of the driving part.

Here, in the surgical instrument 30 according to an embodiment of the present disclosure, when the driving part satellite pulley is moved relative to the driving part relay pulley, which causes the overall length of the jaw wire to be changed in the driving part 200, allowing the end tool 100 to perform a pitch motion. In particular, in the surgical instrument 30 according to an embodiment of the present disclosure, when the driving part pitch pulley 231 is rotated, which causes the driving part satellite pulley to be revolved around the (common) rotation shaft of the driving part relay pulley and the driving part pitch pulley 231 so that a path length of the jaw wire wound around the driving part relay pulley is changed, allowing the end tool to perform a pitch motion.

Specifically, when a motion compensation for the pitch motion is not separately performed in the driving part, the pitch motion itself cannot be performed in the end tool.

Meanwhile, in order for the end tool to perform a pitch motion, the wires 301 and 305 should be further wound around the pulley 113 by ΔSpitch and the wires 302 and 306 should be further unwound from the pulley 114 by ΔSpitch. However, when such compensation is not performed in the driving part, the pitch motion itself cannot be performed in the end tool.

In order to perform motion compensation for the pitch motion as described above, in the surgical instrument 30 according to an embodiment of the present disclosure, the driving part pitch pulleys are rotated while the driving part satellite pulleys are revolved, so that the jaw wires are wound around or released from the driving part relay pulley, which allows the movement of the jaw wires to be compensated for by the rotation of the driving part pitch pulley 231.

In other words, when the pulley 231, which is a driving part pitch pulley, is rotated together with the driving part pitch rotation shaft 243, the driving part satellite pulleys are revolved around the driving part pitch rotation shaft 243. In addition, as the driving part satellite pulleys are revolved around the driving part pitch rotation shaft 243, the jaw wire wound around the driving part relay pulley is changed in length. That is, the jaw wire wound at the end tool 100 side due to the rotation of the pulley 231 is released by the same amount at the driving part 200 side, and the jaw wire unwound at the end tool 100 side is wound by the same amount at the driving part 200 side, so that the pitch motion does not affect the yaw motion.

Viewed from another perspective, when the end tool performs a pitch motion due to the rotation of the driving part pitch pulley 231, the jaw wire (responsible for the yaw and actuation motions) is also moved by the pitch motion. That is, as the pitch rotation is performed around the rotation shaft 143 of the end tool 100, both strands of the jaw wire coupled to one jaw are pulled, and both strands thereof coupled to the other jaw are released. Accordingly, it may be described that in the present disclosure, in order to compensate for the movement of the jaw wire, when the end tool performs the pitch motion, the overall length of the jaw wire in the driving part is changed while the driving part satellite pulley is moved relative to the driving part relay pulley, so that the jaw wire is released (or pulled) at the end tool side as much as the jaw wire is pulled (or released) at the driving part side, thereby compensating for the movement of the jaw wire when the end tool performs the pitch motion.

Hereinafter, the pitch motion will be described in more detail.

When the pulley 231, which is a driving part pitch pulley, is rotated in the direction of an arrow A1 (i.e., in the clockwise direction in the drawing) in order for the pitch motion, the pitch-yaw connector 232 (see FIG. 15) is rotated in the direction of the arrow A1 together with the pulley 231, and thus, the pulleys 219 and 220, which are driving part satellite pulleys fixedly coupled to the pitch-yaw connector 232 (see FIG. 15), are revolved as a whole in the direction of an arrow A2 of FIG. 23A (i.e., in the clockwise direction in the drawing) around the driving part pitch rotation shaft 243 by θ. That is, when the pulley 231 is rotated, the pulleys 219 and 220 are revolved by θ from the position of P1 of FIG. 22A to the position of P2 of FIG. 23A. Viewed from another perspective, it may be described that when the driving part pitch pulley 231 is rotated, the driving part satellite pulley is moved in conjunction with the driving part pitch pulley 231.

At the same time, when the pulley 231, which is a driving part pitch pulley, is rotated in the direction of the arrow A1 (i.e., in the clockwise direction in the drawing), the pitch-yaw connector 232 (see FIG. 15) is rotated in the direction of the arrow A1 together with the pulley 231, and thus, the pulleys 229 and 230, which are driving part satellite pulleys fixedly coupled to the pitch-yaw connector 232 (see FIG. 15), are revolved as a whole in the direction of an arrow A3 of FIG. 23B (i.e., in the clockwise direction in the drawing) around the driving part pitch rotation shaft 243 by θ. That is, when the pulley 231 is rotated, the pulleys 229 and 230 are revolved by θ from the position of P3 of FIG. 22B to the position of P4 of FIG. 23B. Viewed from another perspective, it may be described that when the driving part pitch pulley 231 is rotated, the driving part satellite pulley is moved in conjunction with the driving part pitch pulley 231.

Meanwhile, in this case, the positions of the pulley 215, the pulley 216, the pulley 217, the pulley 218, the pulley 225, the pulley 226, the pulley 227, and the pulley 228, which are driving part relay pulleys coupled to the driving part pitch rotation shaft 243, are not changed. That is, the relative positions of the pulley 211, which is a driving part jaw pulley, the pulley 231, which is a driving part pitch pulley, and the pulley 215, the pulley 216, the pulley 217, and the pulley 218, which are driving part relay pulleys, remain constant. Similarly, the relative positions of the pulley 221, which is a driving part jaw pulley, the pulley 231, which is a driving part pitch pulley, and the pulley 225, the pulley 226, the pulley 227, and the pulley 228, which are driving part relay pulleys, remain constant.

In addition, as described above, the relative position of the driving part satellite pulley with respect to the driving part relay pulley is changed as the driving part satellite pulley is revolved, and thus, the length of each wire wound around the driving part relay pulley, that is, the path length, is changed. Here, since the driving part relay pulley includes the pulley 215, which is a driving part first jaw first relay pulley, and the pulley 217, which is a driving part first jaw second relay pulley, the path length also means the sum of the length of the wire 301 wound around the pulley 215 and the length of the wire 301 wound around the pulley 217 (or, the sum of the length by which the wire 305 is wound around the pulley 216 and the length by which the wire 305 is wound on the pulley 218).

That is, as compared to a path length L1 by which the wires 301 and 305, which are first jaw wires, wound around the driving part relay pulleys at the position of FIG. 22A, a path length L2 by which the first jaw wires wound around the driving part relay pulleys at the position of FIG. 23A is reduced, and thus, the first jaw wires are further released at the driving part 200 side by the reduced path length (L1-L2). That is, the overall lengths of the wires 301 and 305, which are first jaw wires, in the driving part 200 are reduced. In addition, as the overall length of the first jaw wire in the driving part 200 is reduced, the overall length of the first jaw wire in the end tool 100 is increased as much as the first jaw wire is unwound.

In contrast, when the pulley 231, which is a driving part pitch pulley, is rotated in the direction of the arrow A1, as compared to a path length L3 by which the wires 302 and 306, which are second jaw wires, wound around the driving part relay pulleys at the position of FIG. 22B, a path length L4 by which the second jaw wires wound around the driving part relay pulleys at the position of FIG. 23B is increased, and the second jaw wires are further pulled at the driving part 200 side by as much as the increased path length (L4-L3). That is, the overall lengths of the wires 302 and 306, which are second jaw wires, in the driving part 200 are increased. In addition, as the overall length of the second jaw wire in the driving part 200 is increased, the overall length of the second jaw wire in the end tool 100 is reduced as much as the second jaw wire is pulled.

As such, when the pulley 231, which is a driving part pitch pulley, is rotated in the direction of the arrow A1 for a pitch motion, the relative position of the driving part satellite pulley is changed as the driving part satellite pulley is moved relative to the driving part pitch pulley 231 and the driving part relay pulley. In addition, due to the relative movement of the driving part satellite pulley, the overall length of the first jaw wire in the driving part 200 is reduced, and the overall length of the first jaw wire in the end tool 100 is increased. At the same time, due to the relative movement of the driving part satellite pulley, the overall length of the second jaw wire in the driving part 200 is increased, and the overall length of the second jaw wire in the end tool 100 is reduced.

As a result, when the pulley 231, which is a driving part pitch pulley, is rotated in the direction of the arrow A1, the wires 301 and 305, which are two strands of the first jaw wire, are released and the wires 302 and 306, which are two strands of the second jaw wire, are pulled when viewed from the end tool 100 side, so that the end tool 100 performs a pitch motion in the direction of an arrow A4 around the rotation shaft 143.

Here, the term “path length” may be defined as a length of the jaw wire from a point at which the jaw wire enters the driving part first relay pulley to a point at which the jaw wire exits from the driving part second relay pulley through the driving part satellite pulley. That is, the path length may be defined as a length of the wire 301, which is a jaw wire, from a point at which the jaw wire enters the pulley 215, which is a driving part first relay pulley, to a point at which the jaw wire exits from the pulley 217, which is a driving part second relay pulley, through the pulley 219 that is a driving part satellite pulley.

Viewed from another perspective, the path length may be defined as the length of the jaw wire from an initial contact point of the jaw wire with the driving part relay pulley to a final contact point of the jaw wire with the driving part relay pulley on a deployment path of the jaw wire that connects the end tool jaw pulley to the driving part jaw pulley. That is, the path length may be defined as the length of the jaw wire from an initial contact point of the wire 301, which is a jaw wire, with the pulley 215, which is a driving part first relay pulley, to a final contact point of the wire 301 with the pulley 217, which is a driving part second relay pulley.

Meanwhile, as the above-described path length is changed while the driving part satellite pulley is moved relative to the driving part relay pulley, the overall length of the jaw wire in the driving part 200 is also changed. In addition, as the overall length of the jaw wire in the driving part 200 is changed, the overall length of the jaw wire in the end tool 100 is also changed. However, it may be said that since the overall length of the jaw wire in the end tool 100 is also increased (or reduced) by as much as the overall length of the jaw wire increased (reduced) in the driving part 200, a total length of the jaw wire is not changed (assuming that elastic deformation or the like is not considered).

As a result, when the driving part pitch pulley 231 is rotated, the wire 301/wire 305, which are first jaw wires, are released at the driving part 200 side by as much as the wire 301/wire 305, which are first jaw wires, are pulled at the end tool 100 side, as a result, a pitch motion is enabled.

Meanwhile, as described above, the end tool 100 of the surgical instrument 30 of the present disclosure may further include the pulley 131, which is an end tool pitch pulley, the driving part 200 may further include the pulley 231, which is a driving part pitch pulley, and the power transmission part 300 may further include the wire 303 and the wire 304 which are pitch wires.

Accordingly, when the pulley 231, which is a driving part pitch pulley, is rotated in the direction of the arrow A1, due to the rotation of the pulley 231, the wire 304 is wound around the pulley 231 and the wire 303 is released from the pulley 231. Accordingly, the pulley 131, which is an end tool pitch pulley connected to the other sides of the wires 303 and 304, is rotated in the direction of the arrow A2 around the rotation shaft 143, so that the pitch motion may be more surely and reliably performed.

Here, among the pulleys that are rotated around the rotation shaft 143, which is an end tool pitch rotation shaft, the pulley 131, which is an end tool pitch pulley in contact with the wires 303 and 304 that are pitch wires, may be formed to have a diameter different from those of the pulley 113, the pulley 114, the pulley 123, and the pulley 124, which are end tool jaw pitch main pulleys in contact with the wire 301, the wire 305, the wire 302, and the wire 306 that are jaw wires.

In this case, when the rotation shaft 143 is rotated, the lengths of the wires wound around or unwound from the respective pulleys are different from each other. For example, when a diameter of the end tool pitch pulley is 6 φ, a diameter of the end tool jaw pitch main pulley is 4 φ, and the rotation shaft 143 is rotated by 90°, a length of the pitch wire wound around the end tool pitch pulley is 1.5 π, whereas a length of the jaw wire wound around the end tool jaw pitch main pulley may be 1 π.

From this perspective, the length of the wire wound around or unwound from the pulley may be defined as “rotation amount”. The rotation amount is a concept different from a rotation angle, and may be calculated as (diameter*rotation angle/360°*π).

In this case, since essentially the pulley 231, which is a driving part pitch pulley, is directly connected to the pulley 131, which is an end tool pitch pulley, by the wires 303 and 304, which are pitch wires, the rotation amount of the driving part pitch pulley 231 is the same as that of the end tool pitch pulley. That is, the pitch wire is released from or wound around the end tool pitch pulley by as much as the pitch wire is wound around or released from the driving part pitch pulley 231.

Meanwhile, a relation of (diameter of end tool pitch pulley:diameter of end tool jaw pitch main pulley)=(rotation amount of wire wound around end tool pitch pulley:rotation amount of wire wound around end tool jaw pitch main pulley) may be established.

As described above, when, in the end tool 100, the length of the pitch wire wound around the end tool pitch pulley is different from the length of the jaw wire wound around the end tool jaw pitch main pulley, in the driving part 200, the length of the pitch wire to be released should be different from the length of the jaw wire to be released by the same proportion.

To this end, the relationship of (diameter of end tool pitch pulley:diameter of end tool jaw pitch main pulley)=(diameter of driving part pitch pulley:diameter of driving part relay pulley) may be established.

For example, when a ratio of (diameter of end tool pitch pulley:diameter of end tool jaw pitch main pulley) is 6:4, a ratio of (diameter of driving part pitch pulley:diameter of driving part relay pulley) may also be 11:4. According to this ratio, the diameter of the driving part pitch pulley may be 9 φ, and the diameter of the driving part relay pulley may be 6 φ.

However, here, the driving part relay pulley may include two or more pulleys including the driving part first relay pulley and the driving part second relay pulley. In addition, the sum of the diameters of the driving part first relay pulley and the driving part second relay pulley may be defined as the diameter of the driving part relay pulley.

For example, when the diameter of the driving part relay pulley is 6φ, there are several possible combinations for (diameter of driving part first relay pulley, diameter of driving part second relay pulley), including (1φ, 5φ), (2φ, 4φ), (3φ, 3φ), (4φ, 2φ), and (5φ, 1φ), among others. Here, it is illustrated in the drawings that the diameter of the pulley 215, which is a driving part first relay pulley, is 4 φ, and the diameter of the pulley 217, which is the driving part second relay pulley, is 2 φ.

In addition, it may be described that rotation amount of driving part first relay pulley plus the rotation amount of driving part second relay pulley is proportional to the rotation amount of the driving part pitch pulley.

However, although the ratio of (diameter of end tool pitch pulley:diameter of end tool jaw pitch main pulley) may not exactly match the ratio of (diameter of driving part pitch pulley:diameter of driving part relay pulley), when the pulley diameters are selected to make these ratios similar, the object of the present disclosure, which is to compensate for the movement of the jaw wire with the rotation of the driving part pitch pulley, can be achieved to some extent.

The process of the final pitch motion will be described again as follows.

Hereinafter, a case in which the diameter of the end tool pitch pulley is 6 φ, the diameter of the end tool jaw pitch main pulley is 4 φ, the diameter of the driving part pitch pulley is 9 φ, and the diameter of the driving part relay pulley is 6 φ will be described as an example.

First, for a pitch motion, the pulley 231, which is a driving part pitch pulley of the driving part 200, is rotated by 60° to wind the wire 304, which is a pitch wire, while releasing the wire 303. At this time, the length of the wire 303/wire 304 wound and unwound is 1.5 π.

Accordingly, as the wire 304 is pulled by 1.5 π and the wire 303 is released by 1.5 π in the end tool 100, the pulley 131, which is an end tool pitch pulley, is rotated by 90° corresponding to 1.5 π.

Meanwhile, when the pulley 131 is pitch-rotated around the rotation shaft 143, the jaws 101 and 102 and the pulley 111/pulley 112 are also pitch-rotated around the rotation shaft 143. Accordingly, the wires 301 and 305, which are first jaw wires coupled to the pulley 111, are both pulled, and the wires 302 and 306, which are second jaw wires coupled to the pulley 121, are both released. At this time, the angles by which the end tool pitch pulley and the end tool jaw pitch main pulley are rotated are equal to each other and measure 90°, and thus, the length of the jaw wires wound around or released from the end tool jaw pitch main pulley becomes 1 π.

Meanwhile, since the pulley 231 and the pulley 219/pulley 220 are rigidly connected by the pitch-yaw connector 232, when the pulley 231 is rotated by 60° around the driving part pitch rotation shaft 243, the pulley 219/pulley 220 are revolved by 60° around the driving part pitch rotation shaft 243.

In addition, as described above, as the pulley 219/pulley 220 are revolved, the jaw wires are wound around or released from the pulley 215 and the pulley 216, whose combined diameter is 6 φ, by 1 π corresponding to a revolution angle of 60°. That is, the wires 301 and 305, which are first jaw wires, are released as a whole, and the wires 302 and 306, which are second jaw wires, are pulled as a whole.

In other words, the overall path lengths of the wires 301 and 305 wound around the pulley 215, the pulley 216, the pulley 217, and the pulley 218, which are driving part first jaw relay pulleys, are reduced, and the wires 301 and 305 are released by as much as the reduced path length. In addition, the overall path lengths of the wires 302 and 306 wound around the pulley 225, the pulley 226, the pulley 227, and the pulley 228, which are driving part second jaw relay pulleys, are increased, and the wires 302 and 306 are pulled by as much as the increased path length.

That is, the wires 301 and 305, which are first jaw wires, are released at the driving part 200 side by as much as the wires 301 and 305 are pulled at the end tool 100 side, thereby compensating for the movement of the jaw wire due to the pitch motion. Similarly, the wires 302 and 306, which are second jaw wires, are released at the driving part 200 side by as much as the wires 302 and 306 are pulled at the end tool 100 side, thereby compensating for the movement of the jaw wire due to the pitch motion.

As a result, by releasing (or pulling) the jaw wires at the driving part 200 side by as much as a length equal to the length by which the jaw wires are wound around (or released from) the end tool 100 side in response to the pitch motion, the pitch motion can be performed independently without affecting the rotation of the jaw around the yaw shaft.

That is, when the driving part pitch pulley 231 and the driving part satellite pulley are rigidly connected, and the driving part pitch pulley 231 is rotated around the driving part pitch rotation shaft 243, the path length of the jaw wire wound around the driving part relay pulley is changed as the driving part satellite pulley is revolved around the driving part pitch rotation shaft 243. In addition, the change in the path length of the jaw wire compensates for the movement of the jaw wires at the end tool side due to the pitch motion, as a result, the pitch motion is independently performed.

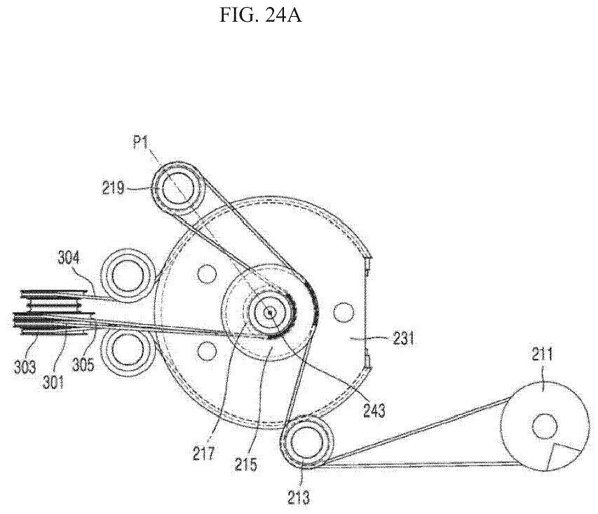

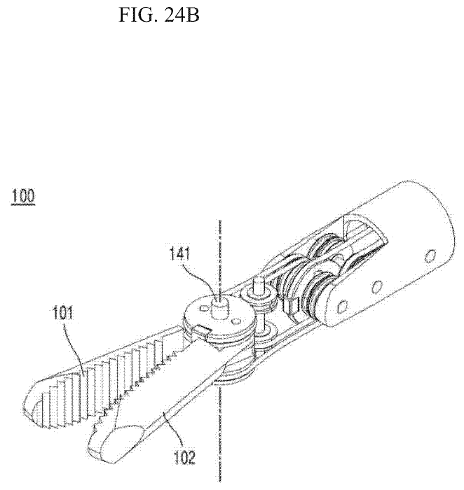

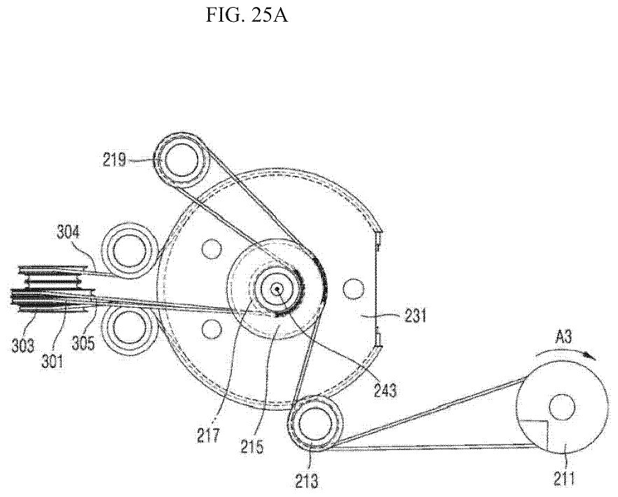

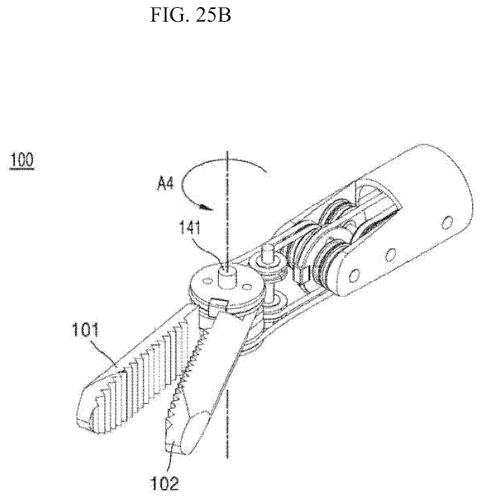

FIGS. 24A to 25B are diagrams illustrating a yaw motion of the surgical instrument illustrated in FIG. 11.

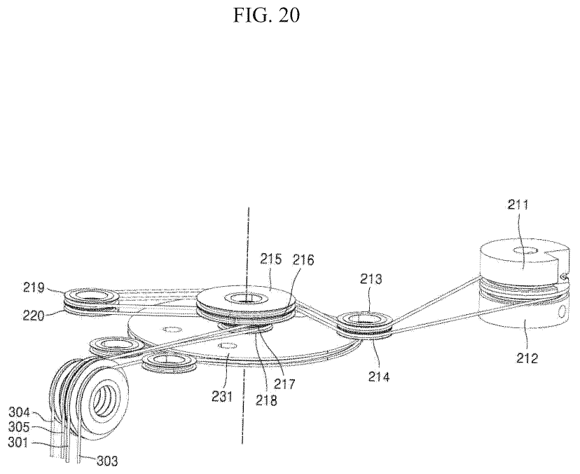

Referring to FIGS. 20, 21, 24A to 25B and the like, when the pulley 211, which is a driving part first jaw pulley, is rotated in the direction of an arrow A3 for a yaw motion, one of the wires 301 and 305, which are first jaw wires, is wound around the pulley 211 and the other one thereof is released from the pulley 211 in response to the rotation of the pulley 211. Accordingly, the pulley 111, which is an end tool first jaw pulley connected to the opposite side of the wires 301 and 305, is rotated in the direction of as arrow A4, so that the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wires 301 and 305 are wound around or released from the driving part satellite pulley and the driving part relay pulley occurs.

Accordingly, the driving part pitch pulley 231 rigidly connected to the driving part satellite pulley is not rotated, and the wires 303 and 304, which are pitch wires, are not wound or released and maintained in position.

Similarly, when the pulley 221, which is a driving part second jaw pulley, is rotated for a yaw motion, in response to the rotation of the pulley 221, one of the wires 302 and 306, which are second jaw wires, is wound around the pulley 221 and the other one thereof is released from the pulley 221. Accordingly, the pulley 121, which is an end tool second jaw pulley connected to the opposite side of the wires 302 and 306, is rotated in one direction, so that the yaw motion is performed.

At this time, the pulley 219, the pulley 220, the pulley 229, and the pulley 230, which are driving part satellite pulleys, and the pulley 215, the pulley 216, the pulley 217, the pulley 218, the pulley 225, the pulley 226, the pulley 227, and the pulley 228, which are driving part relay pulleys, are not changed in position, but only the motion in which the wires 302 and 306 are wound around or released from the driving part satellite pulley and the driving part relay pulley occurs.

Accordingly, the driving part pitch pulley 231 rigidly connected to the driving part satellite pulley is not rotated, and the wires 303 and 304, which are pitch wires, are not wound or released and maintained in position.

As a result, the overall lengths of the wire 301, the wire 302, the wire 305, and the wire 306, which are jaw wires, in the driving part 200 remain constant even when the pulley 211 or pulley 221, which is a driving part jaw pulley, is rotated for the yaw or actuation motion.

As described above, in the surgical instrument 30 according to an embodiment of the present disclosure, when the driving part pitch pulley 231 is rotated, the driving part satellite pulley is revolved around the rotation shaft of the driving part pitch pulley 231 to change the path length of the jaw wire wound around the driving part relay pulley, and the jaw wire is wound or released in response to the rotation of the driving part pitch pulley 231, so that the movement of the jaw wire due to the pitch drive may be offset or compensated, and as a result, the effect of separating the pitch motion and the yaw motion can be obtained.

However, the pitch motion and the yaw motion are not limited to being mechanically separated from each other as described above, and can be separated and performed independently by the processor according to an embodiment of the present disclosure.

### Instrument Carriage Coupling Structure

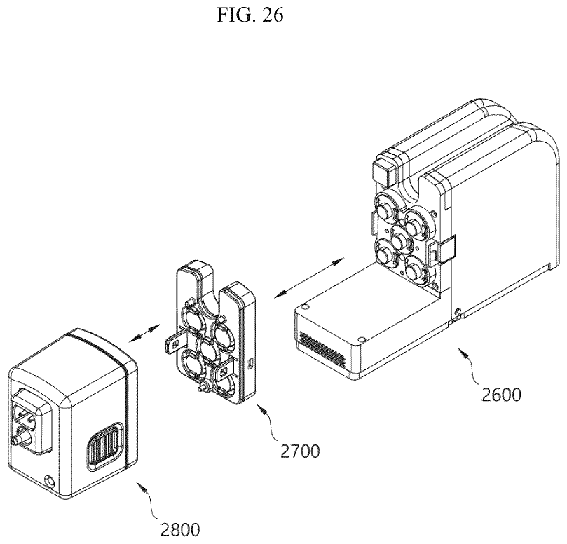

FIG. 26 is an exemplary diagram illustrating a coupling relationship among a surgical instrument carriage, an instrument sterile adapter device, and a surgical instrument driving unit according to an aspect of the present disclosure. As illustrated in FIG. 26, according to an aspect of the present disclosure, the instrument sterile adapter device 2700 may be coupled to the surgical instrument carriage 2600, and the driving unit 2800 of the surgical instrument may be coupled to the instrument sterile adapter device 2700.

In this regard, a minimally invasive surgical instrument needs to perform surgical operations by inserting the instrument into an affected area inside the body of a patient through narrow incisions, and therefore, power from a power source may be transmitted to the end tool through a thin and long tube (for example, shaft) disposed between the area performing the operation (for example, end tool) and the power source. Herein, there is a risk of damage or breakage during a sterilization process when an instrument carriage (for example, motor pack) of the surgical robot that operates the end tool is sterilized with steam, heat, or pressure. Accordingly, a drape covering the surgical robot needs to be utilized to maintain a sterilized state, and since the instrument carriage is related to the operation of the surgical instrument, a sterile adapter utilizing a separate structure is required to be mounted to form a sterilized state. Accordingly, the instrument sterile adapter device capable of smoothly transmitting power to the surgical instrument while covering the instrument carriage with the drape may be utilized. The instrument sterile adapter device is required to have a structure that is easy to detach in order to reduce the surgical setup time.

More specifically, but not limitedly, the robot arm of the slave robot constituting the surgical robot system is provided with the surgical instrument carriage 2600 for mounting the surgical instrument. As described above with reference to the drawings, the surgical instrument carriage 2600 may be configured to be disposed, for example, on an end link of the robot arm and to perform linear motion. As a non-limiting example, the surgical instrument carriage 2600 may be fastened to a slide link of the robot arm and configured to move forward or backward within a range of motion provided by the slide link, thereby controlling the depth at which the surgical instrument coupled to the surgical instrument carriage 2600 is inserted into the body of a patient.

The surgical instrument carriage 2600 may include a motor for operating the surgical instrument, more specifically, the end tool, and may be referred to as, but not limited to, a ‘motor pack’, for example. For a surgical procedure, it is required to ensure sterilization of not only the surgical instrument used in the surgery, but also the slave robot including the motor pack and the robot arm located at a rear side of the motor pack. However, the surgical instrument carriage 2600 includes electronic equipment such as, for example, a motor, and thus, there is a high risk of damage or breakage during the sterilization process when sterilization is performed using steam, heat, or pressure.

To address these issues, a method of maintaining a sterile state by covering the slave robot at a rear side of the surgical instrument carriage 2600 using a drape has been proposed. In particular, since the surgical instrument carriage 2600 is required to maintain a power-transmitting connection relationship with the surgical instrument, a structure in which the instrument sterile adapter device 2700 with the drape fastened thereto is first coupled to the surgical instrument carriage, and then the driving unit 2800 of the surgical instrument is fastened to the instrument sterile adapter device, so that the rotational power is transmitted in the order of the surgical instrument carriage 2600, the instrument sterile adapter device 2700, and the driving unit 2800 of the surgical instrument is adopted.

However, in the conventional fastening structure in which the surgical instrument carriage, the instrument sterile adapter device, and the driving unit of the surgical instrument are coupled in sequence, the fastening structure for ‘coupling between the surgical instrument carriage and the instrument sterile adapter device’ was implemented integrally with the fastening structure for ‘coupling between the instrument sterile adapter device and the driving unit of the surgical instrument,’ which caused various issues.

In other words, in covering the slave robot with a drape by using a separate sterile adapter to sterilize the surgical instrument carriage, and in order to reduce the surgical setup time, there is a requirement that the sterile adapter be easily and quickly mountable on the surgical instrument carriage, that detachment also be convenient, and that attachment and detachment of the surgical instrument likewise be easily and quickly performed. In order to achieve both of these requirements, a single latch structure is adopted to facilitate the mounting of the sterile adapter on the instrument carriage and the mounting of the instrument carriage on the sterile adapter. In other words, the surgical instrument mounting latch and the instrument carriage detachment latch are configured integrally, and a structure is adopted in which a user presses the latch for coupling between the sterile adapter device and the surgical instrument when the surgical instrument is not mounted on the sterile adapter device, thereby detaching the sterile adapter from the instrument carriage. However, since this conventional single latch structure implements the fastening of two portions with one structure, there is an issue in that the shape of the single latch structure needs to withstand all external impacts and loads. Accordingly, since the single latch structure receives all forces applied from the surgical instrument and the instrument carriage, it was very vulnerable to external impacts and loads. In addition, when such a single latch breaks, there is a problem in that it becomes difficult to detach the sterile adapter from the instrument carriage.

The fastening structure connecting the surgical instrument carriage, the instrument sterile adapter device, and the driving unit of the surgical instrument according to an aspect of the present disclosure is intended to address such an issue, and the latch structure for connecting the surgical instrument and the instrument sterile adapter device is used only for fastening between the surgical instrument and the instrument sterile adapter device. The instrument sterile adapter device is not provided with a latch for coupling to the surgical instrument carriage, and a groove for fastening of the fastening structure from the surgical instrument carriage may be provided. The surgical instrument carriage may be provided with a button including a protrusion shape that fits into the groove formed in the instrument sterile adapter device. According to an aspect, such a button may be made of a metal material to have high durability, but it should be noted that the material of the button is not limited to metal.

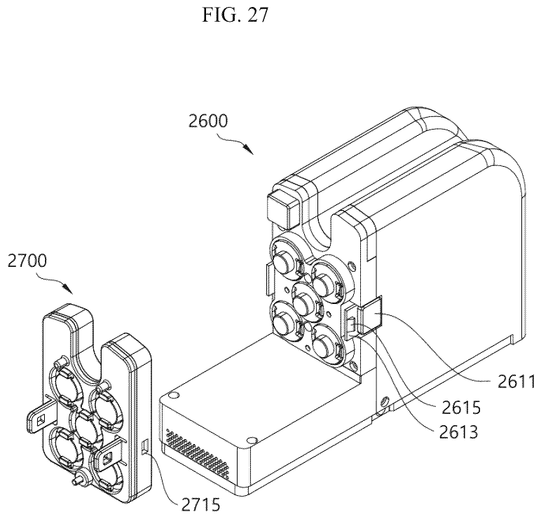

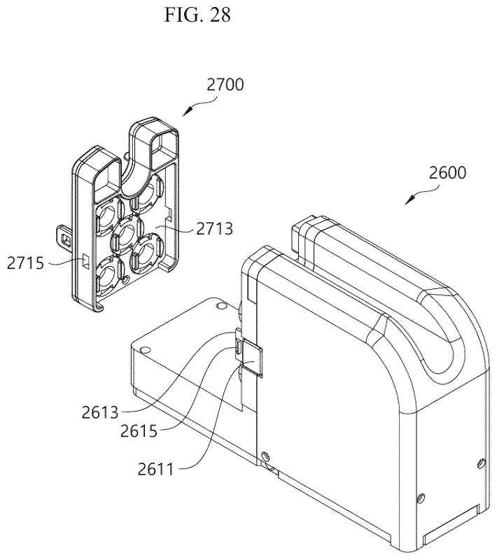

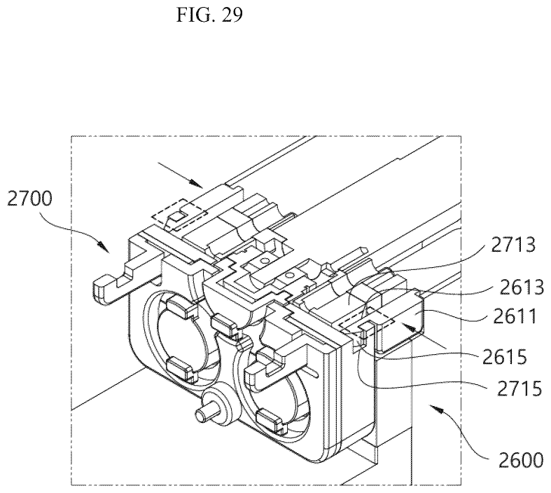

In this regard, FIG. 27 illustrates a coupling relationship between the surgical instrument carriage and the instrument sterile adapter device of FIG. 26 in more detail. FIG. 28 illustrates a coupling relationship between the surgical instrument carriage and the instrument sterile adapter device of FIG. 27 from another direction. In addition, FIG. 29 is an exemplary horizontal cross-sectional view illustrating the surgical instrument carriage and the instrument sterile adapter device of FIG. 27 in a coupled state. Hereinafter, with reference to FIGS. 27 to 29, a non-limiting but more specific description is provided of a fastening structure between the surgical instrument carriage 2600 and the instrument sterile adapter device 2700 according to an aspect of the present disclosure.

As illustrated in FIGS. 27 to 29, the instrument sterile adapter device 2700 according to an aspect of the present disclosure may be detachably coupled to the surgical instrument carriage 2600. More specifically, but not limitedly, the surgical instrument carriage 2600 may be provided with at least one sterile adapter release button 2611, and at least one sterile adapter latch support 2613 may be connected to the sterile adapter release button 2611. The at least one sterile adapter latch support 2613 may be provided with at least one sterile adapter latch 2615 coupled thereto.

In a sterile adapter body forming the instrument sterile adapter device 2700, a carriage coupling surface for coupling to the instrument carriage may be provided with a passage 2713 configured to allow at least one sterile adapter latch support 2613 extending from the instrument carriage 2600 to pass therethrough. For example, as illustrated in FIG. 28, the passage 2713 may be a concave structure formed on the carriage coupling surface of the instrument sterile adapter device 2700, but is not limited thereto. For example, a lumen structure corresponding to the shape of the at least one sterile adapter latch support 2613 may be provided, but is not limited thereto.

The sterile adapter body forming the instrument sterile adapter device 2700 may be provided with a sterile adapter latch receiving groove 2715 in which the at least one sterile adapter latch 2615 coupled to at least one sterilize adapter latch support 2613 is seated, when the sterile adapter body is coupled to the instrument carriage 2600. The sterile adapter latch receiving groove 2715 may be arranged, for example, on a side surface of the sterile adapter body, but is not limited thereto.

According to an aspect, the sterile adapter latch support 2613 as described above may be connected to the sterile adapter release button 2611 provided on the instrument carriage, and may be configured such that the sterile adapter latch 2615 moves away from the sterile adapter latch receiving groove 2715 in response to the sterile adapter release button 2611 being pressed. In other words, by pressing the sterile adapter release button 2611 provided on the surgical instrument carriage 2600 by a user, the sterile adapter latch 2615 may move away from the sterile adapter latch receiving groove 2715, thereby detaching the instrument sterile adapter device 2700 from the surgical instrument carriage 2600.

In other words, the instrument sterile adapter device 2700 may not be provided with a fastening structure for fastening a coupling to the surgical instrument carriage 2600, such as, for example, a conventional single latch structure, and may only be provided with a groove 2715 configured to receive a fastening structure extending from the surgical instrument carriage 2600. Accordingly, the fastening structure for coupling the instrument sterile adapter device 2700 and the surgical instrument carriage 2600 only needs to be able to withstand a force acting on the instrument sterile adapter device 2700 and the surgical instrument carriage 2600, thereby forming a structure that is more resistant to breakage. In addition, even when the fastening structure for coupling to the driving unit 2800 of the surgical instrument provided on the instrument sterile adapter device 2700 is damaged, the instrument sterile adapter device 2700 may be removed from the surgical instrument carriage 2600 without any difficulty.

In other words, according to an aspect of the present disclosure, a fastening structure for coupling between the surgical instrument carriage and the instrument sterile adapter device and a fastening structure for coupling between the instrument sterile adapter device and the surgical instrument may be provided separately. Accordingly, not only can the detachment of the instrument sterile adapter from the surgical instrument carriage be performed quickly and easily, but the instrument sterile adapter device can also be advantageously removed from the surgical instrument carriage even when the fastening structure for coupling with the surgical instrument provided on the instrument sterile adapter device is damaged.

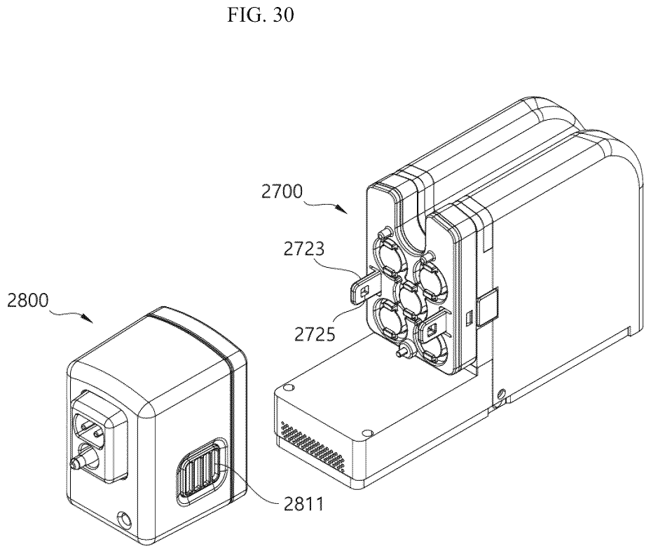

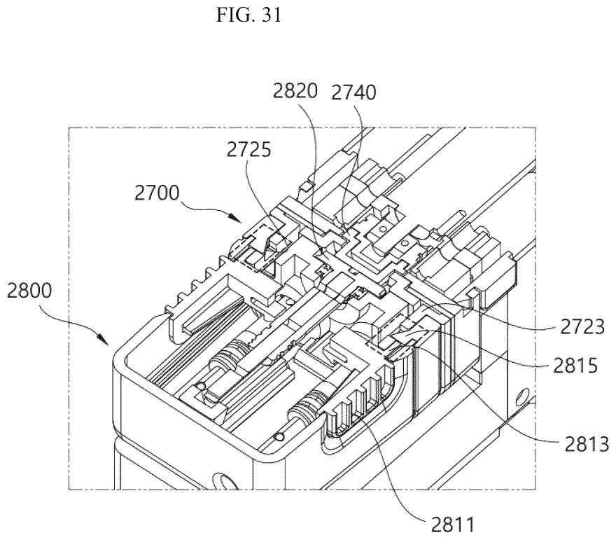

FIG. 30 illustrates a coupling relationship between the instrument sterile adapter device and the surgical instrument driving unit of FIG. 26 in more detail. In addition, FIG. 31 is an exemplary horizontal cross-sectional view of the instrument sterile adapter device and the surgical instrument driving unit of FIG. 30 in a coupled state. Hereinafter, with reference to FIGS. 30 and 31, a non-limiting but more specific description will be given of a coupling structure between the instrument sterile adapter device 2700 and the driving unit 2800 of the surgical instrument according to an aspect of the present disclosure.

As illustrated in FIGS. 30 to 31, the instrument sterile adapter device 2700 according to an aspect of the present disclosure, and more specifically, but not limitedly, the sterile adapter body of the instrument sterile adapter device 2700, may include at least one instrument coupling latch 2723 that protrudes in a direction of the instrument coupling surface for coupling the sterile adapter body and the driving unit 2800 of the surgical instrument. Herein, at least one instrument latch receiving groove 2725 may be formed in the at least one instrument coupling latch 2723.

More specifically, as illustrated in FIGS. 30 and 31, when the driving unit 2800 of the surgical instrument is coupled to the instrument sterile adapter device 2700, at least one instrument latch 2815 provided in the driving unit 2800 of the surgical instrument may be seated in the at least one instrument latch receiving groove 2725 formed in the at least one instrument coupling latch 2723 extended from the instrument sterile adapter device 2700. Herein, the driving unit 2800 of the surgical instrument may be provided with the surgical instrument release button 2811, and the surgical instrument release button 2811 may be coupled to, for example, a button extension support 2813, such that when the surgical instrument release button 2811 is pressed, the button extension support 2813 may also move in a direction of the inside of the driving unit 2800 of the surgical instrument. The movement of the button extension support 2813 in the direction of the inside of the driving unit 2800 of the surgical instrument causes movement of the at least one instrument coupling latch 2723 extended from the instrument sterile adapter device 2700. As a result, the instrument latch 2815 provided in the driving unit 2800 of the surgical instrument may move away from the instrument latch receiving groove 2725 formed in the instrument coupling latch 2723. Accordingly, a user can easily remove the driving unit 2800 of the surgical instrument from the instrument sterile adapter device 2700 by pressing the surgical instrument release button 2811 provided in the driving unit 2800 of the surgical instrument.

### Instrument Sterile Adapter Device Fixed Cover

As described above, a minimally invasive surgical instrument needs to perform surgical operations by inserting instruments into an affected area inside the body of a patient through narrow incisions, and therefore, power from a power source may be transferred to the end tool through a thin and long tube (for example, shaft) disposed between the part performing the operation (for example, an end tool) and the power source. Herein, there is a risk of damage or breakage during a sterilization process when an instrument carriage (for example, motor pack) of the surgical robot that operates the end tool is sterilized with steam, heat, or pressure. Accordingly, a drape covering the surgical robot needs to be utilized to maintain a sterilized state, and since the instrument carriage is related to the operation of the surgical instrument, it is required to mount a sterile adapter having a separate structure to form a sterilized state. Accordingly, the instrument sterile adapter device that may smoothly transfer power to the surgical instrument while covering the instrument carriage with the drape may be utilized.

In other words, since the surgical instrument carriage 2600 is required to maintain a connection capable of transmitting power to the surgical instrument, a structure may be provided in which an instrument sterile adapter device 2700 to which the drape is fastened is first coupled to the surgical instrument carriage, and then the driving unit 2800 of the surgical instrument is fastened to the instrument sterile adapter device, so that the rotational power is transmitted in the order of the surgical instrument carriage 2600, the instrument sterile adapter device 2700, and the driving unit 2800 of the surgical instrument.

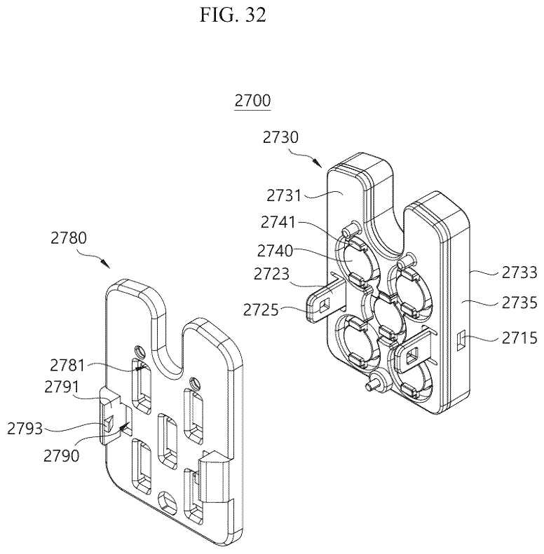

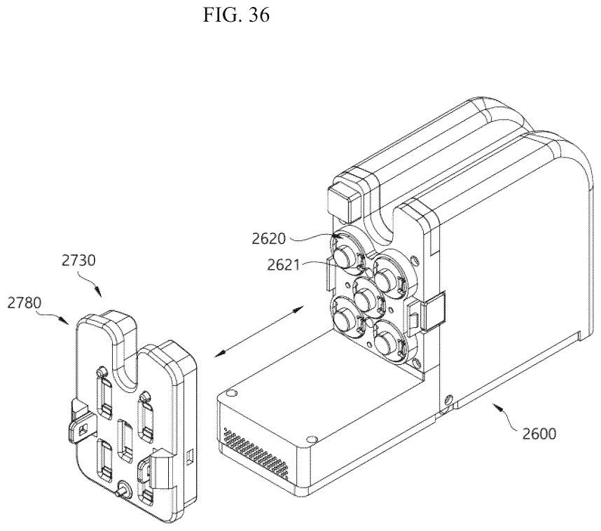

More specifically, but not limitedly, in a state where the surgical instrument carriage 2600, the instrument sterile adapter device 2700, and the driving unit 2800 of the surgical instrument are sequentially coupled to one another, the rotational power from at least one motor provided in the surgical instrument carriage 2600 may be operatively connected to at least one driver (2620 of FIG. 36) provided in the surgical instrument carriage 2600. The rotational power from the at least one driver 2620 may be transmitted to at least one coupler (2740 of FIG. 32) of the instrument sterile adapter device 2700 coupled to the at least one driver. Subsequently, the rotational power from the at least one coupler 2740 may be transmitted to at least one driving pulley (2820 of FIG. 31) of the driving unit 2800 of the surgical instrument coupled to the at least one coupler. Accordingly, depending on the pulley and/or wire connection structure of various embodiments, the rotational movement of the driving pulley 2820 may be converted into a linear movement of a wire, and may cause the operation of at least one driving element of the surgical instrument (for example, rotation of an articulation or opening and closing of a jaw).

In order to achieve such a transmission structure of rotational power, first, when the instrument sterile adapter device 2700 is coupled to the surgical instrument carriage 2600, it is required that the coupling between the at least one driver 2620 of the surgical instrument carriage 2600 and the coupler 2740 of the instrument sterile adapter device 2700 be fastened. In this regard, in order for coupling between the at least one driver 2620 of the surgical instrument carriage 2600 and the coupler 2740 of the instrument sterile adapter device 2700 to be achieved, it is necessary that, while one component is fixed and the other component rotates, a coupling structure for fastening therebetween reaches a corresponding position, and that the coupling between the at least one driver 2620 of the surgical instrument carriage 2600 and the coupler 2740 of the instrument sterile adapter device 2700 be fastened. In this regard, since the at least one driver 2620 of the surgical instrument carriage 2600 is operatively connected with the rotation of the motor provided in the surgical instrument carriage 2600, generally, when the structures for mutual coupling reach corresponding positions while the rotation of the coupler 2740 of the instrument sterile adapter device 2700 is restrained and fixed and the driver 2620 of the surgical instrument carriage 2600 is rotated, the driver 2620 of the surgical instrument carriage 2600 may be coupled to the coupler 2740 of the instrument sterile adapter device 2700. In other words, the coupler 2740 and the driver 2620 may be connected (coupled) while the coupler 2740 is fixed and the driver 2620 is rotated.

To this end, the instrument sterile adapter device 2700 is required to be provided with at least one stopper for restraining the rotation of the at least one coupler 2740. In this regard, conventionally, the instrument sterile adapter device 2700 has been provided with a stopper structure for preventing the rotation of the coupler 2740, thereby preventing the rotation of the coupler 2740 and enabling the coupler 2740 to be coupled to the driver 2620. For example, in the instrument sterile adapter device according to the conventional technology, a groove is formed in the coupler at least at a portion of the circumferential direction, and a stopper for preventing the rotation of the coupler is provided in a body unit of the instrument sterile adapter device. In the case of the instrument sterile adapter device according to the conventional technology, the stopper for preventing the rotation is provided in the sterile adapter device, so that the coupling between the coupler 2740 and the driver 2620 may be easily implemented. However, there was a limit to reducing the width of the sterile adapter device due to the stopper structure disposed in the sterile adapter device itself. The narrower the width of the sterile adapter device, the shorter the length of the surgical robot and/or surgical instrument, which has the advantage of securing the rigidity of each instrument part. In addition, in the case where a stopper is provided to prevent the rotation of the sterile adapter device itself as in the conventional method, there was an issue in that the size of the sterile adapter device might be minimized only when the tolerance between the axial parts of the coupler 2740 and/or the driver 2620 was precisely managed.

According to the conventional method, when the instrument sterile adapter device 2700 is mounted on the surgical instrument carriage 2600, and the surgical instrument carriage 2600 and the instrument sterile adapter device 2700 are coupled to each other and the driver 2620 of the surgical instrument carriage 2600 is rotated, the coupler 2740 of the instrument sterile adapter device 2700 rotates together with the driver 2620 of the surgical instrument carriage 2600 without being fastened thereto. After performing a certain amount of rotation, the coupler 2740 of the instrument sterile adapter device 2700 hits a stopper provided in the instrument sterile adapter device 2700, thereby stopping the rotation of the coupler 2740 and restraining further rotation. Thereafter, only the driver 2620 of the surgical instrument carriage 2600 performs rotation, and when the coupler 2740 and the driver 2620 are coupled, the driver 2620 also ceases to rotate.

Since the conventional structure has a stopper for restraining the rotation of the coupler 2740 provided in the instrument sterile adapter device 2700 itself, as described above, there was a limit to reducing the width and/or size of the instrument sterile adapter device 2700. In addition, there was an issue that the dimensional management of individual parts associated with the coupler 2740 and/or the driver 2620 had to be strictly managed.

The instrument sterile adapter device for the surgical robot system according to an aspect of the present disclosure is intended to address such an issue, and the instrument sterile adapter device 2700 is not provided with a stopper for fixing the coupler 2740, but is not provided with a separate fixed cover, so that the rotation of the coupler 2740 may be restrained by the fixed cover, and the fixed cover may be removed after the coupler 2740 and the driver 2620 are coupled.

For example, according to an aspect of the present disclosure, the sterile adapter body has a carriage coupling surface for coupling to an instrument carriage and an instrument coupling surface disposed opposite to the carriage coupling surface for coupling to a surgical instrument, and the at least one coupler is mounted on the sterile adapter body to transmit rotational power from the instrument carriage to the surgical instrument. Herein, the fixed cover configured to be detachably coupled to the sterile adapter body and to restrain rotation of the at least one coupler while mounted on the sterile adapter body may be provided. Accordingly, since a coupler rotation stopper structure for coupling between a coupler of the instrument sterile adapter device and a driver of the surgical instrument carriage need not be provided in the instrument sterile adapter device, the thickness of the instrument sterile adapter device may be reduced, and thus the length of the surgical instrument and the surgical robot may also be reduced. Accordingly, it has a more advantageous effect in increasing the rigidity of the surgical instrument and the surgical robot. In addition, compared to when the coupler rotation stopper structure is provided on the instrument sterile adapter device, the axial dimensional deviation of the coupler may be more easily managed.

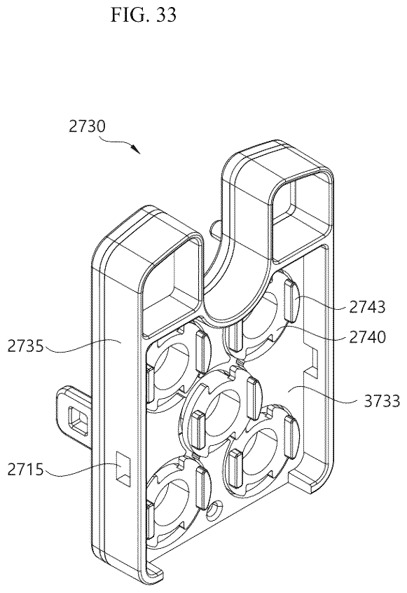

In this regard, FIG. 32 is a schematic perspective view of an instrument sterile adapter device having a fixed cover according to an aspect of the present disclosure. FIG. 33 illustrates the fixed cover of FIG. 32 in a rear view. Hereinafter, the instrument sterile adapter device for the surgical robot system will be described in more detail with reference to FIGS. 32 and 33.

As illustrated in FIG. 32, an instrument sterile adapter device 2700 for the surgical robot system according to one aspect of the present disclosure may include a sterile adapter body 2730, the coupler 2740, and a fixed cover 2780.

The sterile adapter body 2730 may be a main body of the instrument sterile adapter device according to the conventional art that does not have the fixed cover 2780. In other words, the sterile adapter body 2730 may serve as a frame for forming the instrument sterile adapter device 2700. As illustrated in FIG. 32, the sterile adapter body 2730 may be provided with a carriage coupling surface 2733 for coupling to the instrument carriage 2600 and an instrument coupling surface 2731 disposed opposite to the carriage coupling surface for coupling to the surgical instrument (as a non-limiting example, the driving unit 2800 of the surgical instrument). Herein, the carriage coupling surface 2733 may be understood not to mean merely a single flat surface, but rather an area in an opposite direction for coupling the sterile adapter body 2730 to the surgical instrument carriage 2600. Similarly, the instrument coupling surface 2731 may be understood not to mean merely a single flat surface, but rather an area in an opposite direction for coupling the sterile adapter body 2730 to the driving unit 2800 of the surgical instrument. According to an aspect, the surface connecting the carriage coupling surface 2733 and the instrument coupling surface 2731 of the sterile adapter body 2730 may be referred to as a side surface of the sterile adapter body 2730.

Referring again to FIG. 32, the sterile adapter body 2730 may be provided with at least one coupler 2740 for transmitting the rotational power from the instrument carriage 2600 to the driving unit 2800 of the surgical instrument. The coupler 2740 may be mounted on the sterile adapter body 2730 and may be disposed to be rotatable about a central axis along a coupling direction in which the surgical instrument carriage 2600, the instrument sterile adapter device 2700, and the driving unit 2800 of the surgical instrument are coupled. In addition, one surface of the coupler 2740 may be exposed on the carriage coupling surface 2733, and the other surface of the coupler 2740 may be exposed on the instrument coupling surface 2731.

As illustrated in FIG. 32, according to an aspect of the present disclosure, the instrument sterile adapter device 2700 is provided with the fixed cover 2780. The fixed cover 2780 may be detachably coupled to the sterile adapter body 2730. With the fixed cover 2780 mounted on the sterile adapter body 2730, the fixed cover 2780 may be configured to restrain the rotation of the at least one coupler 2740.

According to an aspect, the instrument sterile adapter device 2700 may be coupled to the instrument carriage 2600 in a state where the fixed cover 2780 mounted on the sterile adapter body 2730 of the instrument sterile adapter device 2700. Thereafter, the fixed cover 2780 may be removed after at least one driver 2620 of the instrument carriage is coupled to at least one coupler 2740.

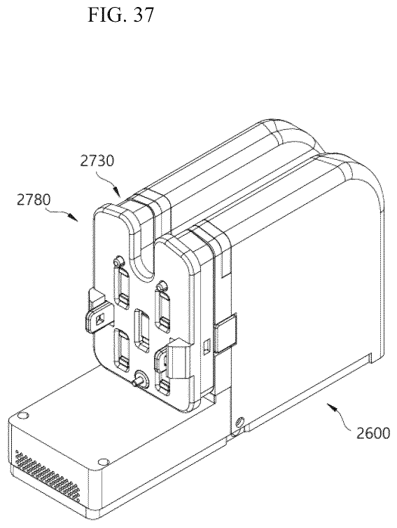

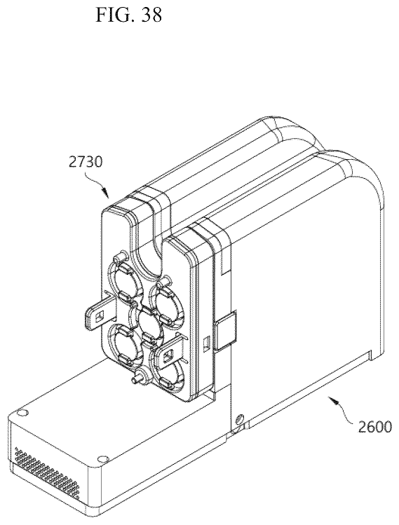

In this regard, FIG. 36 illustrates an exemplary preparation state for coupling to the surgical instrument carriage when the fixed cover of FIG. 32 is coupled to the sterile adapter body. FIG. 37 illustrates an exemplary state of coupling to the surgical instrument carriage when the fixed cover of FIG. 32 is coupled to the sterile adapter body. FIG. 38 illustrates an exemplary state in which the fixed cover is detached in the coupled state of FIG. 37.

As illustrated in FIGS. 36 and 37, the instrument sterile adapter device 2700 may be coupled to the instrument carriage 2600 with the fixed cover 2780 mounted on the sterile adapter body 2730 of the instrument sterile adapter device 2700. As illustrated in FIG. 36, at least one driver 2620 may be provided in a direction toward the instrument sterile adapter device 2700 of the surgical instrument carriage 2600. In a state where the instrument sterile adapter device 2700 is coupled to the surgical instrument carriage 2600, the driver 2620 may be coupled to the coupler 2740 of the instrument sterile adapter device 2700 while rotating as described above. When the coupling of the driver 2620 and the coupler 2740 is completed, the fixed cover 2780 is detached from the sterile adapter body 2730 as illustrated in FIG. 38, so that the driving unit 2800 of the surgical instrument may be mounted on the sterile adapter body 2730 of the instrument sterile adapter device 2700.

Referring again to FIGS. 33 and 36, the at least one coupler 2740 may be provided with at least one driver coupling unit 2743 disposed in a direction of the carriage coupling surface 2733, and the at least one driver (2620 of FIG. 36) of the instrument carriage 2600 may be provided with a coupler coupling unit (2621 of FIG. 36) disposed in a direction toward the instrument sterile adapter device 2700. As illustrated in FIGS. 33 and 36, for example, the driver coupling unit 2743 may have a protruding shape, and the coupler coupling unit 2621 may have a groove shape. However, the technical idea of the present disclosure is not limited thereto, and it should be understood that any structure capable of achieving coupling of the two configurations is included in the technical idea of the present disclosure.

When the surgical instrument carriage 2600 and the instrument sterile adapter device 2700 are coupled, the driver coupling unit 2743 and the coupler coupling unit 2621 may be coupled by rotating the at least one driver 2620 so that the positions of the driver coupling unit 2743 and the coupler coupling unit 2621 correspond to each other in the rotational direction. Herein, the at least one driver 2620 of the instrument carriage may be rotated by at least one motor (not shown) provided in the instrument carriage, but the present disclosure is not limited thereto.

Referring again to FIG. 32, the at least one coupler 2740 according to an aspect of the present disclosure may be provided with at least one stopper coupling unit disposed in a direction of the instrument coupling surface 2731, and the fixed cover 2780 according to an aspect of the present disclosure may be provided with the at least one stopper disposed in a direction toward the instrument sterile adapter device and configured to restrain the rotation of the coupler.

As a non-limiting example, as illustrated in FIG. 32, the at least one stopper coupling unit may be at least one protruding stopper coupling unit 2741 protruding in a direction toward the instrument coupling surface 2731. In addition, as illustrated in FIG. 32, the at least one stopper may be at least one groove-shaped stopper 2781 that accommodates the at least one protruding stopper coupling unit 2741.

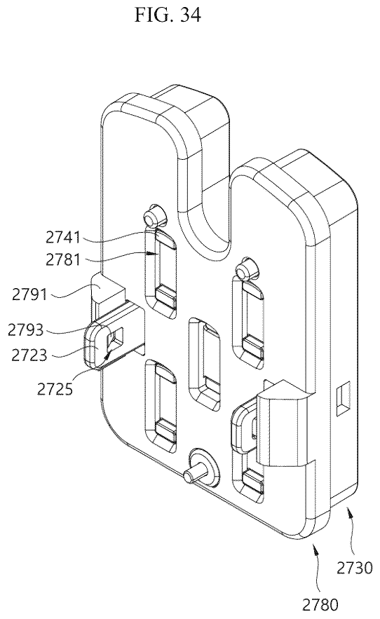

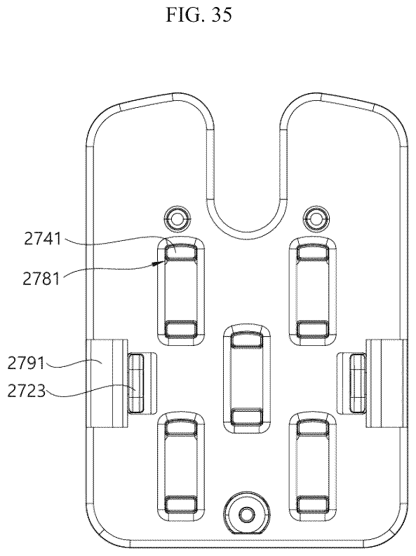

In this regard, FIG. 34 is an exemplary perspective view of the fixed cover of FIG. 32 and the sterile adapter body in a coupled state. FIG. 35 is an exemplary front view of the fixed cover of FIG. 32 and the sterile adapter body in a coupled state. As illustrated in FIGS. 34 and 35, in a state where the fixed cover 2780 is coupled to the sterile adapter body 2730, the at least one protruding stopper coupling unit 2741 provided in the sterile adapter body 2730 may be seated in the at least one groove-shaped stopper 2781 provided in the fixed cover 2780. Accordingly, in a state where the fixed cover 2780 is coupled to the sterile adapter body 2730, the rotational movement of the at least one coupler 2740 is restrained, thereby assisting the coupling of the driver 2620 of the surgical instrument carriage 2600 and the coupler 2740.

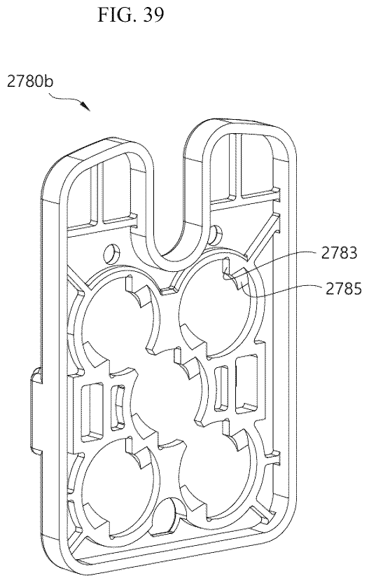

FIG. 39 illustrates an exemplary rear view of a fixed cover according to another embodiment of the present disclosure. As illustrated in FIG. 39, instead of being provided with the groove-shaped stopper 2781, a fixed cover 2780b according to another embodiment of the present disclosure may be provided with a step portion 2783 and a tapered portion 2785 on a rear surface of the fixed cover 2780b, for example, on a surface of the fixed cover 2780b facing the sterile adapter body 2730. More specifically, but not limited thereto, the step portion 2783 may be disposed at a position corresponding to a first position in the rotational direction of the coupler, and the tapered portion 2785 may be disposed such that a height is gradually reduced from the step portion along the rotational direction of the coupler. Accordingly, in a state where the fixed cover 2780b is coupled to the sterile adapter body 2730, a first direction rotation of the coupler 2740 is permitted by allowing the protruding stopper coupling unit 2741 formed in the coupler 2740 of the sterile adapter body 2730 as illustrated in FIG. 32 to move along the tapered portion 2785, but a second direction rotation of the coupler 2740 opposite to the first direction may be restrained by allowing the protruding stopper coupling unit 2741 to be caught by the step portion 2783. Accordingly, for coupling the driver 2620 of the surgical instrument carriage 2600 and the coupler 2740 of the instrument sterile adapter device 2700, the driver 2620 may be rotated, for example, in a second direction.

Although not illustrated in the drawing, it should be understood that various forms of stopper coupling units and embodiments of the stopper are included in the technical idea of the present disclosure. For example, the at least one stopper coupling unit may include at least one groove-shaped stopper coupling unit formed at least in a direction of the instrument coupling surface 2731. In other words, the groove-shaped stopper coupling unit may be formed over the entire width direction of the coupler, or may be formed only in a portion of the width direction in the direction of the instrument coupling surface 2731. In addition, the at least one stopper may be at least one protruding stopper inserted into the at least one groove-shaped stopper coupling unit. In other words, the rotational movement of the coupler 2740 may be restrained by the protruding stopper extending from the fixed cover 2780 toward the sterile adapter body 2730 being seated in the groove-shaped stopper coupling unit formed in the coupler 2740.

In addition, according to an aspect, the at least one stopper coupling unit may be at least one driving pulley coupling unit for coupling to at least one driving pulley (2820 of FIG. 31) of the surgical instrument in a state where the driving unit 2800 of the surgical instrument is coupled to the instrument sterile adapter device 2700. In other words, as a non-limiting example, according to an aspect, the fixed cover 2780 may fix a protrusion unit formed in the coupler 2740 to be coupled to the driving pulley 2820 of the surgical instrument based on the groove-shaped stopper 2781 to restrain the rotation of the coupler 2740. Herein, the at least one driving pulley 2820 provided in the driving unit 2800 of the surgical instrument may be configured to control at least one operation of the surgical instrument based on rotational power from the at least one driver 2620 transferred through the coupler 2740.

Referring again to FIGS. 32, 34, and 35, the fixed cover 2780 and the sterile adapter body 2730 may further include a fastening structure for coupling therebetween. According to an aspect, such a fastening structure may utilize a fastening structure formed on the sterile adapter body 2730 for coupling the sterile adapter body 2730 to the driving unit 2800 of the surgical instrument.

As a non-limiting example, as illustrated in FIG. 32, the sterile adapter body 2730 may include the at least one instrument coupling latch 2723 protruding in the direction of the instrument coupling surface 2731. Herein, at least one instrument latch receiving groove 2725 may be formed in the at least one instrument coupling latch, in which at least one instrument latch (2815 of FIG. 31) provided on the driving unit 2800 of the surgical instrument is seated in a state where the driving unit 2800 of the surgical instrument is coupled to the instrument sterile adapter device 2700.

As illustrated in FIG. 32, the fixed cover 2780 according to an aspect may include at least one instrument coupling latch hole 2790 configured to allow at least one instrument coupling latch to pass through. In addition, the fixed cover 2780 may include at least one fixed cover latch 2793 seated in the instrument latch receiving groove 2725 in a state where the fixed cover is coupled to the sterile adapter body. In this regard, the fixed cover 2780 may further include at least one fixed cover latch support 2791 extending in a direction opposite to the sterile adapter body, and the fixed cover latch 2793 may be formed on the fixed cover latch support 2791.

As illustrated in FIGS. 34 to 35, when the fixed cover 2780 is coupled to the sterile adapter body 2730, the instrument coupling latch 2723 of the sterile adapter body 2730 may be inserted into the instrument coupling latch hole 2790 formed in the fixed cover 2780. Herein, the fixed cover latch 2793 coupled to the fixed cover latch support 2791 extending from the fixed cover 2780 in a direction opposite to the sterile adapter body 2730 is seated in the instrument latch receiving groove 2725 formed in the instrument coupling latch 2723, so that the fixed cover 2780 and the sterile adapter body 2730 may be fastened to each other. As illustrated in FIG. 34, the at least one instrument coupling latch 2723 may have a length such that it protrudes further than the fixed cover latch support 2791 in a state where the fixed cover is coupled to the sterile adapter body. Accordingly, a user may release a coupling of the fixed cover 2780 and the sterile adapter body 2730 by moving the instrument coupling latch 2723 in the direction spaced apart from the fixed cover latch support 2791.

Although explained above with reference to the drawings or embodiments, it does not mean that the scope of protection of the present disclosure is limited by the drawings or embodiments, and it should be understood that a person skilled in the art can variously modify and change the present disclosure within a scope not deviating from the idea and area of the present disclosure as recited in the following claims.

Although the above-mentioned present disclosure is explained based on a series of functional blocks, it is not limited by the aforementioned embodiments and attached drawings. Additionally, it would be obvious to a person skilled in the art to which the present disclosure pertains that various substitutions, modifications and changes are possible within a scope not deviating the technical idea of the present disclosure.

A combination of the above-mentioned embodiments is not limited to the aforementioned embodiments, and various types of combinations may be provided as well as the aforementioned embodiments according to implementation and/or necessity.

In the above-mentioned embodiments, the methods are explained based on a flow chart with a series of steps or blocks, but the present disclosure is not limited to the order of the steps, and some steps may be performed in a different order with other steps other than the above, or may be performed at the same time. Also, a person skilled in the art would understand that the steps in the flow chart are not exclusive, other steps can be included, or one or more steps in the flow chart can be deleted without affecting the scope of the present disclosure.

The above-mentioned embodiments include various aspects of examples. Although all possible combinations to express various aspects cannot be described, a person skilled in the art would recognize that other combinations are possible. Therefore, the present disclosure should include all other substitutions, modifications, and variations falling within the scope of the following claims.

What is claimed is:

1. An instrument sterile adapter device for a surgical robot system, the device comprising:
a sterile adapter body having a carriage coupling surface and an instrument coupling surface, wherein the carriage coupling surface is configured to be coupled to an instrument carriage, and the instrument coupling surface is disposed opposite to the carriage coupling surface and configured to be coupled to a surgical instrument;
at least one coupler mounted on the sterile adapter body, and configured to transfer rotational power from the instrument carriage to the surgical instrument; and
a fixed cover detachably coupled to the sterile adapter body and configured to restrain rotation of the at least one coupler while the fixed cover is mounted on the sterile adapter body.

2. The device of claim 1, wherein:
the instrument sterile adapter device is configured to be coupled to the instrument carriage in a state where the fixed cover is mounted on the instrument sterile adapter device; and
the fixed cover is configured to be removed after at least one driver of the instrument carriage is coupled to the at least one coupler.

3. The device of claim 2, wherein:
the at least one coupler includes at least one driver coupling unit protruded from the carriage coupling surface;
the at least one driver of the instrument carriage includes at least one coupler coupling unit disposed correspondingly to the at least one driver coupling unite; and
the at least one driver coupling unit and the at least one coupler coupling unit are configured to be mutually engaged by rotation of the at least one driver so as to correspond to each other.

4. The device of claim 3, wherein the at least one driver of the instrument carriage configured to be rotated by at least one motor provided in the instrument carriage.

5. The device of claim 1, wherein:
the at least one coupler includes at least one stopper coupling unit protruded from the instrument coupling surface; and
the fixed cover includes at least one stopper disposed correspondingly to the at least one stopper coupling unit configured to restrain rotation of the coupler.

6. The device of claim 5, wherein the at least one stopper coupling unit comprises at least one protruding stopper coupling unit protruding from the instrument coupling surface.

7. The device of claim 6, wherein the at least one stopper comprises at least one groove-shaped stopper configured to accommodate the at least one protruding stopper coupling unit.

8. The device of claim 6, wherein the at least one stopper comprises:
a step portion formed at a first position in a rotational direction of the coupler; and
a tapered portion having a height gradually decreasing from the step portion along the rotational direction of the coupler,
wherein the at least one protruding stopper coupling unit is configured to allow a first direction rotation of the coupler by moving along the tapered portion, and to restrain a second direction rotation of the coupler opposite to the first direction by causing the at least one protruding stopper coupling unit to be caught by the step portion.

9. The device of claim 5, wherein:
the at least one stopper coupling unit comprises at least one groove-shaped stopper coupling unit; and
the at least one stopper comprises at least one protruding stopper configured to be inserted into the at least one groove-shaped stopper coupling unit.

10. The device of claim 5, wherein the at least one stopper coupling unit is at least one driving pulley coupling unit configured to be coupled to at least one driving pulley of the surgical instrument in a state where the surgical instrument is coupled to the instrument sterile adapter device.

11. The device of claim 10, wherein the at least one driving pulley configured to control an operation of the surgical instrument based on a rotational power of at least one driver, transmitted through the coupler.

12. The device of claim 1, wherein:
the sterile adapter body comprises at least one instrument coupling latch protruded from the instrument coupling surface; and
the at least one instrument coupling latch has at least one instrument latch receiving groove formed such that at least one instrument latch provided on the surgical instrument is seated in a state where the surgical instrument is coupled to the instrument sterile adapter device.

13. The device of claim 12, wherein the fixed cover comprises:
at least one instrument coupling latch hole configured to allow the at least one instrument coupling latch to pass through; and
at least one fixed cover latch seated in the instrument latch receiving groove in a state where the fixed cover is coupled to the sterile adapter body.

14. The device of claim 13, wherein:
the fixed cover further comprises at least one fixed cover latch support extending in a direction opposite to the sterile adapter body; and
the at least one fixed cover latch is formed on the at least one fixed cover latch support.

15. The device of claim 14, wherein the at least one instrument coupling latch has a length such that the at least one instrument coupling latch protrudes further than the at least one fixed cover latch support in a state where the fixed cover is coupled to the sterile adapter body.

16. The device of claim 1, wherein the sterile adapter body comprises:
a passage through which at least one sterile adapter latch support extending from the instrument carriage passes through the carriage coupling surface; and
a sterile adapter latch receiving groove in which at least one sterile adapter latch coupled to the at least one sterile adapter latch support is seated in a state where the sterile adapter body is coupled to the instrument carriage.

17. The device of claim 16, wherein the sterile adapter latch receiving groove is formed on a side surface of the sterile adapter body.

18. The device of claim 16, wherein the sterile adapter latch support is connected to a sterile adapter release button provided on the instrument carriage, and is configured such that the sterile adapter latch disengages from the sterile adapter latch receiving groove in response to a pressing of the sterile adapter release button.
